(19) United States
(12) Patent Application Publication
(10) Pub. No.: US 2009/0270371 A1
(43) Pub. Date: Oct. 29, 2009

(54) QUINOLINE DERIVATIVES USEFUL IN THE TREATMENT OF MGLUR5 RECEPTOR-MEDIATED DISORDERS

(76) Inventors: Gyorgy Keseru, Telki (HU); Csaba Weber, Budapest (HU); Attila Bielik, Orkeny (HU); Amrita Agnes Bobok, Budapest (HU); Krisztina Gal, Budapest (HU); Marta Meszlenyine Sipos, Budapest (HU); Laszlo Molnar, Budapest (HU); Monika Vastag, Budapest (HU)

Correspondence Address: FISH & RICHARDSON P.C., PO BOX 1022, MINNEAPOLIS, MN, 55440-1022, US

(21) Appl. No.: 12/158,596

(22) PCT Filed: Dec. 19, 2006
(86) PCT No.: PCT/HU06/00121

§ 371 Date: Nov. 10, 2008

(30) Foreign Application Priority Data
Dec. 20, 2005 (HU) P0501165
Dec. 18, 2006 (HU) P0600918

(51) Int. Cl. A61K 31/4709; C07D 413/04; C07D 401/04; C07D 215/38; C07D 491/056; A61K 31/5377; A61K 31/47; A61K 31/496; A61K 31/4741; A61K 31/55; A61P 25/00

(52) U.S. Cl. 514/217.07; 544/128; 546/153; 546/90; 544/363; 514/235.2; 514/312; 514/253.07; 514/291

(57) ABSTRACT

Compounds of formula (I): and/or enantiomers and/or racemates and/or diastereomers and/or pharmaceutically acceptable salts thereof formed with acids or bases, to the process for their preparation, to the intermediates of the preparation process, to the pharmaceutical formulations containing these compounds and to their use in the prevention and/or treatment of mGluR5 receptor-mediated disorders.

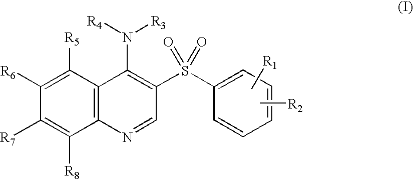

### FIELD OF THE INVENTION

The present invention relates to a new class of compounds, to the process for their preparation, to the intermediates of the preparation process, to the pharmaceutical formulations containing these compounds and to their use in the prevention and/or treatment of mGluR5 receptor-mediated disorders.

### BACKGROUND OF THE INVENTION

A major excitatory neurotransmitter in the mammalian central nervous system (CNS) is the glutamate molecule, which binds to neurons, thereby activating cell surface receptors. These receptors can be divided into two major classes, ionotropic and metabotropic glutamate receptors, based on the structural features of the receptor proteins.

Metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A2; increases in arachidonic acid release; and increases or decreases in the activity of voltage- and ligand-gated ion channels. (Schoepp et al., Trends Pharmcol. Sci. 1993, 14:13; Schoepp, Neurochen. Int. 1994, 24:439, Pin et al., Neuropharmacology 1995, 34:1; Bordi and Ugolini, Prog. Neurobiol. 1999, 59:55).

Eight distinct mGluR subtypes, termed mGluR1 through mGluR8, have been identified by molecular cloning (Nakanishi, Neuron 1994, 13:1031; Pin et al., Neuropharmacology 1995, 34:1; Knopfel et al., J. Med. Chem. 1995, 38:1417). Further receptor diversity occurs via expression of alternatively spliced forms of certain mGluR subtypes (Pin et al., PNAS 1992, 89:10331; Minakami et al., BBRC 1994, 199:1136, Joly et al., J. Neurosci. 1995, 15:3970).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants.

Attempts at elucidating the physiological roles of Group I mGluRs suggest that activation of these receptors elicits neuronal excitation. Evidence indicates that this excitation is due to direct activation of postsynaptic mGluRs, but it also has been suggested that activation of presynaptic mGluRs occurs, resulting in increased neurotransmitter release (Pin et al., Neuropharmacology 1995, 34:1; Watkins et al., Trends Pharmacol. Sci. 1994, 15:33).

Metabotropic glutamate receptors have been implicated in a number of normal processes in the mammalian CNS. Activation of mGluRs has been shown to be required for induction of hippocampal long-term potentiation and cerebellar long-term depression (Bashir et al., Nature 1993, 363:347; Bortolotto et al., Nature 1994, 368:740; Aiba et al., Cell 1994, 79:365; Aiba et al., Cell 1994, 79:377). A role for mGluR activation in nociception and analgesia also has been demonstrated (Meller et al., Neuroreport 1993, 4: 879; Bordi and Ugolini, Brain Res. 1999, 871:223).

Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease, acute and chronic pain, substance abuse and withdrawal, obesity and gastroesophageal reflux disease (GERD) (Schoepp et al., Trends Pharmacol. Sci. 1993, 14:13; Cunningham et al., Life Sci. 1994, 54:135; Hollman et al., Ann. Rev. Neurosci. 1994, 17:31; Pin et al., Neuropharmacology 1995, 34:1; Knopfel et al., J. Med. Chem 1995, 38:1417; Spooren et al., Trends Pharmacol. Sci. 2001, 22:331; Gasparini et al. Curr. Opin. Pharmacol. 2002, 2:43; Neugebauer Pain 2002, 98:1, Slassi et al., Curr Top Med. Chem. 2005; 5(9):897-911). MGluR5-selective compounds such as 2-methyl-6-(phenylethynyl)-pyridine (“MPEP”) are effective in animal models of mood disorders, including anxiety and depression (Spooren et al., J. Pharinacol. Exp. Ther. 2000, 295:1267; Tatarczynska et al., Br. J. Pharmacol. 2001, 132:1423; Klodzynska et al., Pol. J. Pharmacol, 2001, 132:1423). Much of the pathology in these conditions is thought to be due to excessive glutamate-induced excitation of CNS neurons. As Group I mGluRs appear to increase glutamate-mediated neuronal excitation via postsynaptic mechanisms and enhanced presynaptic glutamate release, their activation probably contributes to the pathology. Therefore, selective antagonists of Group I mGluR receptors could be therapeutically beneficial, especially as neuroprotective agents, analgesics or anticonvulsants.

Various derivatives of compounds that have the above mentioned pharmacological activity are described in the literature.

International Patent Application WO 02/20489 relates to new quinoline derivatives and methods of their use for the treatment of cGMP-associated conditions, cardiovascular disorders, sexual dysfunction, diabetes and gastrointestinal disorders. The compounds are stated to be particularly potent and selective inhibitors of cGMP PDE-5.

International Patent Application WO 03/080580 describes novel quinolin compounds and their use in the treatment of anxiety, depression, obesity and cognitive memory disorders. These compounds are stated to be useful as 5-HT6 receptor antagonists. They are useful for treating neurological diseases and disorders.

International Patent Application WO 05/058834 relates to novel quinoline derivatives for use in treating liver X receptor (LXR) mediated diseases particularly multiple sclerosis, rheumatoid arthritis, inflammatory bowel disease and atherosclerosis, which compounds suppress Th-1 type lymphokine production, resulting in increased HDL levels, and cholesterol metabolism.

However there remains a need for novel compounds and composition that exhibit an activity at metabotropic glutamate receptors (mGluRs), especially at the mGluR5 receptor.

### SUMMARY OF THE INVENTION

Compounds of the present invention are represented by formula (I):

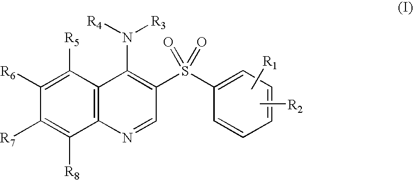

wherein
R₁ and R₂ are independently selected from the group consisting of hydrogen, halogen, alkyl, alkoxy, cyano, optionally substituted amino group or saturated heterocyclyl group, wherein the heteroatom is N;
R₃ and R₄ are independently selected from the group consisting of hydrogen, alkyl, substituted aryl group having at least one substituent selected from the group of hydrogen, halogen, alkyl, alkoxy group, or
R₃ and R₄ together with the N atom to which they are attached can form C₅₋₇ heterocyclyl group, containing 1 or 2 heteroatom(s) selected from the group of N, O, which may be optionally substituted by the groups selected from hydrogen, halogen, alkyl, alkylhydroxy, alkyloxycarbonyl, aminocarbonyl, —OCH₂CH₂O—, benzyl and substituted phenyl group;
R₅, R₆, R₇ and R₅ are independently selected from the group consisting of hydrogen, halogen, alkyl, alkoxy, cyano group, or
R₆ and R₇ together with the atoms to which they are attached can form an unsaturated heterocyclyl group;
and/or enantiomers and/or racemates and/or diastereomers and/or pharmaceutically acceptable salts thereof formed with acids or bases.

Another aspect of the present invention provides processes for the synthesis of compounds of formula (I).

A further aspect of the present invention relates to the intermediates of the preparation process.

A further aspect of the present invention provides pharmaceutical compositions containing a therapeutically effective amount of a compound of formula (I) and/or enantiomers and/or racemates and/or diastereomers and/or salts and/or hydrates or solvates thereof as active ingredient and pharmaceutically acceptable diluents, excipients and/or inert carriers.

A further aspect of the present invention provides the use of a compound of formula (I) for the prevention and/or treatment of mgluR5 receptor mediated disorders, particularly neurological disorders, psychiatric disorders, acute and chronic pain and neuromuscular dysfunctions of the lower urinary tract and gastrointestinal disorders.

A further aspect of the present invention provides the use of a compound of formula (I) for the manufacture of a medicament for the prevention and/or treatment of mGluR5 receptor-mediated disorders, particularly neurological disorders, psychiatric disorders, acute and chronic pain and neuromuscular dysfunctions of the lower urinary tract and gastrointestinal disorders.

A further aspect of the present invention provides methods of prevention and/or treatment of mGluR5 receptor-mediated disorders with a compound of formula (I), which means administering to a mammal to be treated—including human—effective amount/amounts of compounds of formula (I) of the present invention as such or as medicament.

These and other aspects of the present invention are described in detail herein.

### DETAILED DESCRIPTION OF THE INVENTION

Compounds of the present invention are represented by formula (I):

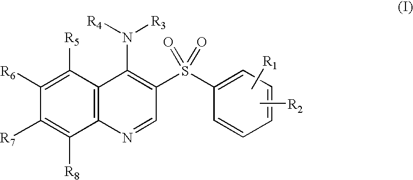

wherein
R₁ and R₂ are independently selected from the group consisting of hydrogen, halogen, alkyl, alkoxy, cyano, optionally substituted amino group or saturated heterocyclyl group, wherein the heteroatom is N;
R₃ and R₄ are independently selected from the group consisting of hydrogen, alkyl, substituted aryl group having at least one substituent selected from the group of hydrogen, halogen, alkyl, alkoxy group, or
R₃ and R₄ together with the N atom to which they are attached can form C₅₋₇ heterocyclyl group, containing 1 or 2 heteroatom(s) selected from the group of N, O, which may be optionally substituted by the groups selected from hydrogen, halogen, alkyl, alkylhydroxy, alkyloxycarbonyl, aminocarbonyl, —OCH₂CH₂O—, benzyl and substituted phenyl group;
R₅, R₆, R₇ and R₅ are independently selected from the group consisting of hydrogen, halogen, alkyl, alkoxy, cyano group, or
R₆ and R₇ together with the atoms to which they are attached can form an unsaturated heterocyclyl group;
and/or enantiomers and/or racemates and/or diastereomers and/or pharmaceutically acceptable salts thereof formed with acids or bases.

More preferred compounds of this invention include compounds of formula (I), wherein R₁ and R₂ are independently selected from the group consisting of hydrogen, halogen, C₁₋₄ alkyl, C₁₋₄ alkoxy, cyano, optionally substituted amino group or saturated heterocyclyl group, wherein the heteroatom is N;

R₃ and R₄ are independently selected from the group consisting of hydrogen, C₁₋₄ alkyl, substituted aryl group having at least one substituent selected from the group of hydrogen, halogen, C₁₋₄ alkyl, C₁₋₄ alkoxy group, or
R₃ and R₄ together with the N atom to which they are attached can form C₅₋₇ heterocyclyl group, containing 1 or 2 heteroatom(s) selected from the group of N, O, which may be optionally substituted by the groups selected from hydrogen, halogen, C₁₋₄ alkyl, C₁₋₄ alkylhydroxy, alkyloxycarbonyl, aminocarbonyl, —OCH₂CH₂O—, benzyl and substituted phenyl group, which may be optionally substituted by 1 or 2 groups selected from hydrogen, halogen, C₁₋₄ alkyl, C₁₋₄ alkoxy group;
R₅, R₆, R₇ and R₉ are independently selected from the group consisting of hydrogen, halogen, C₁₋₄ alkyl, C₁₋₄ alkoxy, cyano group, or
R₆ and R₇ together with the atoms to which they are attached can form an unsaturated 5 to 7 membered heterocyclyl group, containing 1 or 2 O atom(s);
and/or enantiomers and/or racemates and/or diastereomers and/or pharmaceutically acceptable salts thereof formed with acids or bases.

Another more preferred embodiment includes those compounds of formula (I), wherein R¹ and R² are independently selected from the group consisting of hydrogen, chloro, fluoro, C₁₋₂ alkyl, C₁₋₂ alkoxy, cyano or piperidinyl group;

R₃ and R₄ are independently selected from the group consisting of hydrogen, C₁₋₂ alkyl, benzyl group substituted 1 or 2 groups independently selected from hydrogen, halogen, C₁₋₄ alkyl, C₁₋₄ alkoxy group,
R₃ and R₄ together with the N atoms to which they are attached can form pyrrolidinyl, homopiperidinyl, morpholinyl group or piperidinyl group which may be optionally substituted by the groups selected from hydrogen, halogen, C₁₋₄ alkyl, hydroxymethyl, alkyloxycarbonyl, aminocarbonyl, and —OCH₂CH₂O— group or piperazinyl group which may be substituted at N(4) by the groups selected from C₁₋₄ alkyl, benzyl, -alkyloxycarbonyl and phenyl, which may be optionally substituted by 1 or 2 groups selected from hydrogen, halogen, C₁₋₄ alkyl, alkoxy group;
R₅, R₆, R₇ and R₉ are independently selected from the group consisting of hydrogen, chloro, fluoro, C₁₋₂ alkyl, C₁₋₂ alkoxy, cyano group, or
R₆ and R₇ together with the atoms to which they are attached can form 2,3-dihydro-[1,4]dioxine or 2,5-dihydro-furan ring;
and/or enantiomers and/or racemates and/or diastereomers and/or pharmaceutically acceptable salts thereof formed with acids or bases.

Listed below are definitions of various terms used in the specification and claims to describe the present invention.

For the avoidance of doubt it is to be understood that where in this specification a group is qualified by hereinbefore defined, defined hereinbefore or defined above the said group encompasses the first occurring and broadest definition as well as each and all of the other definitions for that group.

For the avoidance of doubt it is to be understood that in this specification “C₁₋₄” means a carbon containing linear or branched group having 1, 2, 3 or 4 carbon atoms.

As used herein the term “alkyl” as well as other groups having the prefix “alk” such as alkoxy means carbon chains which may be linear or branched or combinations thereof. Examples of alkyl groups include methyl, ethyl, propyl, isopropyl, butyl, sec- and tert-butyl, pentyl, hexyl, heptyl and the like.

The term “hetero” unless specifically stated otherwise includes one or more O or N atoms. For example heterocyclic ring systems that contain one or more O or N atoms in the ring, including mixtures of such atoms. Heteroatoms replace carbon atoms. Examples of heterocyclic rings include pyrrolidinyl, piperidinyl, piperazinyl, morpholinyl and piperidin-2-on groups.

The term “halogen” includes fluorine, chlorine, bromine and iodine atoms.

The term “amino group” unless specifically stated otherwise includes —NH₂ group and groups, derived from primary or secondary amines, which also can mean aromatic amines.

The term “optionally substituted” is intended to include both substituted and unsubstituted groups.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Among the salts formed with bases especially important are the salts formed with alkali metals, e.g. sodium, potassium, alkali-earth metals, e.g. calcium and magnesium, as well as with ammonia or organic amines. The latter bases can have further substituents, e.g. hydroxy or amino groups, which can influence e.g. the solubility and the handling of the product. Both organic and inorganic acids can be used for the formation of acid addition salts. Suitable inorganic acids can be e.g. hydrochloric acid, sulfuric acid and phosphoric acid. Representatives of monovalent organic acids can be e.g. formic acid, acetic acid, trifluoroacetic acid, propionic acid, and different butyric acids, valeric acids and capric acids. Representatives of bivalent organic acids can be e.g. oxalic acid, malonic acid, maleic acid, fumaric acid and succinic acid. Other organic acids can also be used, such as hydroxy acids e.g. citric acid, tartaric acid, or aromatic carboxylic acids e.g. benzoic acid or salicylic acid, as well as aliphatic and aromatic sulfonic acids e.g. methanesulfonic acid and p-toluenesulfonic acid. Especially valuable group of the acid addition salts is in which the acid component itself does not have therapeutical effect in the applied dose or it does not have unfavorable influence on the effect of the active ingredient. These acid addition salts are pharmaceutically acceptable acid addition salts. The reason why acid addition salts, which do not belong to the pharmaceutically acceptable acid addition salts belong to the present invention is, that in given case they can be advantageous in the purification and isolation of the desired compounds.

Compounds described herein can contain one or more asymmetric centers and may thus give rise to diastereomers and optical isomers. The present invention includes all such possible diastereomers as well as their racemic mixtures and their substantially pure enantiomers.

Especially important compounds of formula (I) of the present invention are the following:
3-(3,4-Dimethyl-benzenesulfonyl)-4-(morpholin-4-yl)quinoline
3-(4-Methyl-benzenesulfonyl)-4-(3-methyl-piperidin-1-yl)-quinoline
3-(3,4-Dimethyl-benzenesulfonyl)-4-(4-methyl-piperidin-1-yl)-quinoline
3-(4-Methyl-benzenesulfonyl)-4-(4-methyl-piperidin-1-yl)-quinoline
3-Benzenesulfonyl-4-(piperidin-1-yl)-quinoline
3-(4-Methyl-benzenesulfonyl)-4-(piperidin-1-yl)-quinoline
4-Benzylamino-3-(4-methyl-benzenesulfonyl)-quinoline
6-Ethyl-4-(4-methyl-piperidin-1-yl)-3-(4-methoxy-benzenesulfonyl)-quinoline
6-Fluoro-3-(4-methyl-benzenesulfonyl)-4-(4-methyl-piperidin-1-yl)-quinoline
6-Ethoxy-3-(4-chloro-benzenesulfonyl)-4-(4-fluoro-benzylamino)-quinoline
4-(Azepan-1-yl)-3-(4-methyl-benzenesulfonyl)-quinoline
4-(Azepan-1-yl)-3-(4-chloro-benzenesulfonyl)-quinoline
6-Methyl-3-(4-methyl-benzenesulfonyl)-4-(4-methyl-piperidin-1-yl)-quinoline
4-(4-Methylpiperidin-1-yl)-3-benzenesulfonyl-quinoline
9-(4-methyl-piperidin-1-yl)-8-benzenesulfonyl-2,3-dihydro-[1,4]dioxino[2,3-g]quinoline
6-Ethyl-4-(4-ethyloxycarbonyl-piperidin-1-yl)-3-(4-chloro-benzenesulfonyl)-quinoline
4-Diethylamino-3-(4-methyl-benzenesulfonyl)-quinoline
4-(4-Benzyl-piperazin-1-yl)-3-(4-chloro-benzenesulfonyl)-quinoline
4-(Azepan-1-yl)-3-benzenesulfonyl-quinoline
3-(3-Cyano-benzenesulfonyl)-6-fluoro-4-(4-methyl-piperidin-1-yl)-quinoline
6-Fluoro-3-(4-methoxy-benzenesulfonyl)-4-(4-methyl-piperidin-1-yl)-quinoline
6-Fluoro-3-(3-methoxy-benzenesulfonyl)-4-(4-methyl-piperidin-1-yl)-quinoline
6-Fluoro-3-(3,4-dimethyl-benzenesulfonyl)-4-(4-methyl-piperidin-1-yl)-quinoline
3-(3-Chloro-4-methoxy-benzenesulfonyl)-6-fluoro-4-(4-methyl-piperidin-1-yl)-quinoline
3-(3-Chloro-4-fluoro-benzenesulfonyl)-6-fluoro-4-(4-methyl-piperidin-1-yl)-quinoline
3-(3,4-Dichloro-benzenesulfonyl)-6-fluoro-4-(4-methyl-piperidin-1-yl)-quinoline
3-(3-Chloro-benzenesulfonyl)-6-fluoro-4-(4-methyl-piperidin-1-yl)-quinoline
3-(4-Chloro-benzenesulfonyl)-6-methyl-4-(4-methyl-piperidin-1-yl)-quinoline
3-(3-Fluoro-benzenesulfonyl)-6-methyl-4-(4-methyl-piperidin-1-yl)-quinoline
3-(3-Methoxy-benzenesulfonyl)-6-methyl-4-(4-methyl-piperidin-1-yl)-quinoline
3-(3,4-Dimethyl-benzenesulfonyl)-6-methyl-4-(4-methyl-piperidin-1-yl)-quinoline
3-(3-Chloro-4-methoxy-benzenesulfonyl)-6-methyl-4-(4-methyl-piperidin-1-yl)-quinoline
3-(3-Chloro-benzenesulfonyl)-6-methyl-4-(4-methyl-piperidin-1-yl)-quinoline
3-(3-Fluoro-4-methyl-benzenesulfonyl)-6-methyl-4-(4-methyl-piperidin-1-yl)-quinoline
3-(3-Chloro-4-methyl-benzenesulfonyl)-6-methyl-4-(4-methyl-piperidin-1-yl)-quinoline
3-(3-Chloro-4-fluoro-benzenesulfonyl)-6-methyl-4-(4-methyl-piperidin-1-yl)-quinoline
3-(3,4-Dichloro-benzenesulfonyl)-7-fluoro-4-(4-methyl-piperidin-1-yl)-quinoline
7-Fluoro-3-(3-cyano-benzenesulfonyl)-4-(4-methyl-piperidin-1-yl)-quinoline
7-Fluoro-3-(4-cyano-benzenesulfonyl)-4-(4-methyl-piperidin-1-yl)-quinoline
3-(3-Chloro-4-methyl-benzenesulfonyl)-7-fluoro-4-(4-methyl-piperidin-1-yl)-quinoline
7-Fluoro-3-(3-methoxy-benzenesulfonyl)-4-(4-methyl-piperidin-1-yl)-quinoline
3-(3,4-Difluoro-benzenesulfonyl)-7-fluoro-4-(4-methyl-piperidin-1-yl)-quinoline
3-(3-chloro-4-fluoro-benzenesulfonyl)-7-fluoro-4-(4-methyl-piperidin-1-yl)-quinoline
7-Chloro-3-(3,5-dichloro-benzenesulfonyl)-4-(4-methyl-piperidin-1-yl)-quinoline
7-Chloro-3-(3,5-difluoro-benzenesulfonyl)-4-(4-methyl-piperidin-1-yl)-quinoline
7-Chloro-3-(3-cyano-benzenesulfonyl)-4-(4-methyl-piperidin-1-yl)-quinoline
7-Chloro-3-(4-methyl-benzenesulfonyl)-4-(4-methyl-piperidin-1-yl)-quinoline
7-Chloro-3-(4-fluoro-benzenesulfonyl)-4-(4-methyl-piperidin-1-yl)-quinoline
3-Benzenesulfonyl-7-Chloro-4-(4-methyl-piperidin-1-yl)-quinoline
7-Chloro-3-(4-chloro-benzenesulfonyl)-4-(4-methyl-piperidin-1-yl)-quinoline
7-Chloro-3-(3,4-dimethoxy-benzenesulfonyl)-4-(4-methyl-piperidin-1-yl)-quinoline
7-Chloro-3-(3-fluoro-benzenesulfonyl)-4-(4-methyl-piperidin-1-yl)-quinoline
7-Chloro-3-(3-methoxy-benzenesulfonyl)-4-(4-methyl-piperidin-1-yl)-quinoline
7-Chloro-3-(3-chloro-4-methoxy-benzenesulfonyl)-4-(4-methyl-piperidin-1-yl)-quinoline
7-Chloro-3-(3-chloro-benzenesulfonyl)-4-(4-methyl-piperidin-1-yl)-quinoline
7-Chloro-3-(3-chloro-4-fluoro-benzenesulfonyl)-4-(4-methyl-piperidin-1-yl)-quinoline
6-Chloro-3-(3,5-difluoro-benzenesulfonyl)-4-(4-methyl-piperidin-1-yl)-quinoline
6-Chloro-3-(4-methyl-benzenesulfonyl)-4-(4-methyl-piperidin-1-yl)-quinoline
6-Chloro-3-(4-chloro-benzenesulfonyl)-4-(4-methyl-piperidin-1-yl)-quinoline
8-Fluoro-3-(3-fluoro-4-methyl-benzenesulfonyl)-4-(4-methyl-piperidin-1-yl)-quinoline
8-Fluoro-3-(4-methyl-benzenesulfonyl)-4-(4-methyl-piperidin-1-yl)-quinoline
3-(3,4-Dichloro-benzenesulfonyl)-8-fluoro-4-(4-methyl-piperidin-1-yl)-quinoline
3-(3-chloro-4-fluoro-benzenesulfonyl)-8-fluoro-4-(4-methyl-piperidin-1-yl)-quinoline
3-(3,4-Difluoro-benzenesulfonyl)-8-fluoro-4-(4-methyl-piperidin-1-yl)-quinoline
3-Benzenesulfonyl-6-methyl-4-(morpholin-1-yl)-quinoline
3-(3-Chloro-benzenesulfonyl)-6-methoxy-4-(morpholin-1-yl)-quinoline
3-Benzenesulfonyl-6-fluoro-4-(morpholin-1-yl)-quinoline
6-Chloro-3-(4-chloro-benzenesulfonyl)-4-(morpholin-1-yl)-quinoline
3-(3-Chloro-4-methyl-benzenesulfonyl)-7-fluoro-4-(morpholin-1-yl)-quinoline
37-(3,4-Dichloro-benzenesulfonyl)-7-fluoro-4-(morpholin-1-yl)-quinoline
7-Chloro-3-(3,5-dichloro-benzenesulfonyl)-4-(morpholin-1-yl)-quinoline
7-Chloro-3-(3,4-dimethyl-benzenesulfonyl)-4-(morpholin-1-yl)-quinoline
7-Chloro-3-(3-chloro-benzenesulfonyl)-4-(morpholin-1-yl)-quinoline
7-Chloro-3-(3-chloro-4-methyl-benzenesulfonyl)-4-(morpholin-1-yl)-quinoline
7-Chloro-3-(3,4-dichloro-benzenesulfonyl)-4-(morpholin-1-yl)-quinoline
7-Chloro-3-(3-chloro-4-fluoro-benzenesulfonyl)-4-(morpholin-1-yl)-quinoline.

Pharmaceutical Formulations

The present invention provides in a further aspect pharmaceutical compositions comprising a compound represented by formula (I) and/or enantiomers and/or racemates and/or diastereomers and/or pharmaceutically acceptable salts and/or hydrates and/or solvates thereof as active ingredient and one or more pharmaceutically acceptable carriers.

The compounds of formula (I) and/or enantiomers and/or racemates and/or diastereomers and/or pharmaceutically acceptable salts and/or hydrates and/or solvates thereof may be administered by any convenient method, for example by oral, parenteral (including subcutaneous, intramuscular, and intravenous), buccal, sublingual, nasal, rectal or transdermal administration and the pharmaceutical compositions adapted accordingly.

The compounds of formula (I) and/or enantiomers and/or racemates and/or diastereomers and/or pharmaceutically acceptable salts and/or hydrates and/or solvates thereof which are active when given orally can be formulated as liquids or solids, for example syrups, suspensions or emulsions, tablets, capsules and lozenges.

A liquid formulation of the compounds of formula (I) and/or enantiomers and/or racemates and/or diastereomers and/or pharmaceutically acceptable salts and/or hydrates and/or solvates thereof generally consist of a suspension or solution of the compound of formula (I) and/or enantiomers and/or racemates and/or diastereomers and/or pharmaceutically acceptable salts and/or hydrates and/or solvates thereof in a suitable liquid carrier(s) for example an aqueous solvent, such as water, ethanol or glycerine, or a non-aqueous solvent, such as polyethylene glycol or an oil. The formulation may also contain a suspending agent, preservative, flavouring or colouring agent.

A composition in the solid form of a tablet can be prepared using any suitable pharmaceutical carrier(s) routinely used for preparing solid formulations. Examples of solid carriers include lactose, terra alba, sucrose, talcum, gelatine, agar, pectin, acacia, magnesium stearate, stearic acid etc. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent.

A composition in the solid form of a capsule can be prepared using routine encapsulation procedures. For example, pellets containing the active ingredient can be prepared using standard carriers and then filled into a hard gelatine capsule; alternatively, a dispersion or suspension can be prepared using any suitable pharmaceutical carrier(s), for example aqueous gums, celluloses, silicates or oils and the dispersion or suspension then filled into a soft gelatine capsule.

Typical parenteral compositions consisting of a solution or suspension of the compound of formula (I) and/or enantiomers and/or racemates and/or diastereomers and/or pharmaceutically acceptable salts and/or hydrates and/or solvates thereof in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions of the present invention for nasal administration containing a compound of formula (I) and/or enantiomers and/or racemates and/or diastereomers and/or salts thereof may conveniently be formulated as aerosols, drops, gels and powders. Aerosol formulations of the present invention typically comprise a solution or fine suspension of the compound of formula (I) and/or enantiomers and/or racemates and/or diastereomers and/or pharmaceutically acceptable salts and/or hydrates and/or solvates thereof in a pharmaceutically acceptable aqueous or non-aqueous solvent and are usually presented in a single or multidose quantities in sterile form is a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas, such as compressed air or an organic propellant, such as a fluorochlorohydrocarbon. The aerosol dosages form can also take the form of a pump-atomiser.

Compositions of the present invention containing a compound of formula (I) and/or enantiomers and/or racemates and/or diastereomers and/or pharmaceutically acceptable salts and/or hydrates and/or solvates are suitable for buccal or sublingual administration including tablets, lozenges and pastilles, wherein the active ingredient is formulated with a carrier, such as sugar and acacia, tragacanth, or gelatine, glycerin etc.

Compositions of the present invention containing a compound of formula (I) and/or enantiomers and/or racemates and/or diastereomers and/or pharmaceutically acceptable salts and/or hydrates and/or solvates thereof for rectal administration are conveniently in the form of suppositories containing a conventional suppository base, such as cocoa butter and other materials commonly used in the art. The suppositories may be conveniently formed by first admixing the composition with the softened or melted carrier(s) followed by chilling and shaping in moulds.

Compositions of the present invention containing a compound of formula (I) and/or enantiomers and/or racemates and/or diastereomers and/or pharmaceutically acceptable salts and/or hydrates and/or solvates thereof for transdermal administration include ointments, gels and patches.

The compositions of the present invention containing a compound of formula (I) and/or enantiomers and/or racemates and/or diastereomers and/or pharmaceutically acceptable salts and/or hydrates and/or solvates thereof is preferably in the unit dose form, such as tablet, capsule or ampoule.

Each dosage unit of the present invention for oral administration contains preferably from 0.1 to 500 mg of a compound of formula (I) and/or enantiomers and/or racemates and/or diastereomers and/or pharmaceutically acceptable salts and/or hydrates and/or solvates thereof calculated as a free base.

Each dosage unit of the present invention for parenteral administration contains preferably from 0.1 to 500 mg of a compound of formula (I) and/or enantiomers and/or racemates and/or diastereomers and/or pharmaceutically acceptable salts and/or hydrates and/or solvates thereof calculated as a free base.

The pharmaceutically acceptable compounds of formula (I) and/or enantiomers and/or racemates and/or diastereomers and/or pharmaceutically acceptable salts and/or hydrates and/or solvates thereof can be administered in a daily dosage regimen. In the treatment of mGluR5 mediated disorders, such as schizophrenia, anxiety, depression, panic, bipolar disorders, and circadian disorders or chronic and acute pain disorders the dosage levels from about 0.01 mg/kg to about 140 mg/kg of body weight per day are useful or alternatively about 0.5 mg to about 7 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 25-300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Medical Use

The compounds of formula (I) and/or enantiomers and/or racemates and/or diastereomers and/or pharmaceutically acceptable salts and/or hydrates and/or solvates of the present invention have been found to exhibit biological activity at mGluR5 receptors and are expected to be useful in the treatment of mGluR5 mediated disorders.

It has been found that the compounds according to the present invention or salts thereof, exhibit a high degree of potency and selectivity for individual metabotropic glutamate receptor (mGluR) subtypes. In particular there are compounds according to the present invention that are potent and selective for mGluR5 receptor. Accordingly, the compounds of the present invention are expected to be useful in the prevention and/or treatment of conditions associated with excitatory activation of an mGluR5 receptor and for inhibiting neuronal damage caused by excitatory activation of an mGluR5 receptor. The compounds may be used to produce an inhibitory effect of mGluR5, in mammals, including human.

Thus, it is expected that the compounds of the invention are well suited for the prevention and/or treatment of mGluR5 receptor-mediated disorders such as acute and chronic neurological and psychiatric disorders, chronic and acute pain disorders.

The compounds of the present invention are also well suited for the treatment of neuromuscular dysfunction of the lower urinary tract, such as urinary urgency, overactive bladder, greater urinary frequency, reduced urinary compliance, cystitis, incontinence, enuresis and dysuria.

The dose required for the therapeutic or preventive treatment of a particular disorder will necessarily be varied depending on the host treated and the route of administration.

The invention relates to compounds of formula (I) and/or enantiomers and/or racemates and/or diastereomers and/or pharmaceutically acceptable salts and/or hydrates and/or solvates thereof as defined hereinbefore, for use in therapy.

The invention relates to compounds of formula (I) as defined hereinbefore, for use in prevention and/or treatment of mGluR5 receptor-mediated disorders.

The invention relates to compounds of formula (I) as defined hereinbefore, for use in prevention and/or treatment of neurological disorders.

The invention relates to compounds of formula (I) as defined hereinbefore, for use in prevention and/or treatment of psychiatric disorders.

The invention relates to compounds of formula (I) as defined hereinbefore, for use in prevention and/or treatment of chronic and acute pain disorders.

The invention relates to compounds of formula (I) as defined hereinbefore, for use in prevention and/or treatment of neuromuscular dysfunctions of the lower urinary tract and gastrointestinal disorders.

The invention relates to compounds of formula (I) as defined hereinbefore, for use in prevention and/or treatment of pain related to migraine, inflammatory pain, neuropathic pain disorders such as diabetic neuropathies, arthritis and rheumatoid diseases, low back pain, post-operative pain and pain associated with various conditions including angina, in renal or biliary colic, menstruation, migraine and gout.

The invention relates to compounds of formula (I) as defined hereinbefore, for use in prevention and/or treatment of Alzheimer's disease senile dementia, AIDS-induced dementia Parkinson's disease, amyotrophic lateral sclerosis, Huntington's Chorea, migraine, epilepsy, schizophrenia, depression, anxiety, acute anxiety, obesity, obsessive compulsive disorder, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula (I) as defined hereinbefore, for use in prevention and/or treatment of stroke, head trauma, anoxic and ischemic injuries, hypoglycemia, cardiovascular diseases and epilepsy.

The compounds are also well suited for the treatment of neuromuscular dysfunction of the lower urinary tract, such as urinary urgency, overactive bladder, greater urinary frequency, reduced urinary compliance, cystitis, incontinence, enuresis and dysuria.

The compounds are also well suited for the treatment of gastrointestinal disorders, such as transient lower esophageal sphincter relaxation (TLESR), gastrointestinal reflux disease and irritable bowel syndrome.

The present invention relates also to the use of a compound of formula (I) as defined hereinbefore, in the manufacture of a medicament for the prevention and/or treatment of mGluR5 receptor-mediated disorders and any disorder listed above.

The invention also provides a method of treatment and/or prevention of mGluR5 receptor mediated disorders and any disorder listed above, in a patient suffering from, or at risk of, said condition, which comprises administering to the patient an effective amount of a compound of formula (I), as hereinbefore defined.

In the context of the present specification, the term “therapy” includes treatment as well as prevention, unless there are specific indications to the contrary. The terms “therapeutic” and “therapeutically” should be construed accordingly.

In this specification, unless stated otherwise, the term “antagonist” means a compound that by any means, partly or completely blocks the transduction pathway leading to the production of a response by the ligand.

The term “disorder”, unless stated otherwise, means any condition and disease associated with metabotropic glutamate receptor activity.

Methods of Preparation

### Abbreviations

The abbreviations used herein have the following tabulated meanings. Abbreviations not tabulated below have their meanings as commonly used unless specifically stated otherwise.

### DMF N,N-dimethylformamide

t-BuOH 2-methyl-2-propanol
AcOH acetic acid
THF tetrahydrofuran

According to the present invention a process for the preparation of a compound of formula (I):

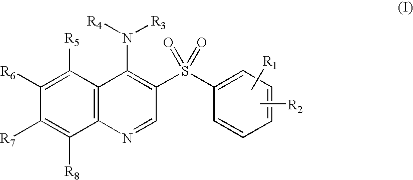

wherein
R₁ and R₂ are independently selected from the group consisting of hydrogen, halogen, alkyl, alkoxy, cyano, optionally substituted amino group or saturated heterocyclyl group, wherein the heteroatom is N;
R₃ and R₄ are independently selected from the group consisting of hydrogen, alkyl, substituted aryl group having at least one substituent selected from the group of hydrogen, halogen, alkyl, alkoxy group, or
R₃ and R₄ together with the N atom to which they are attached can form C₅₋₇ heterocyclyl group, containing 1 or 2 heteroatom(s) selected from the group of N, O, which may be optionally substituted by the groups selected from hydrogen, halogen, alkyl, alkylhydroxy, alkyloxycarbonyl, aminocarbonyl, —OCH₂CH₂O—, benzyl and substituted phenyl group;
R₅, R₆, R₇ and R₈ are independently selected from the group consisting of hydrogen, halogen, alkyl, alkoxy, cyano group, or
R₆ and R₇ together with the atoms to which they are attached can form an unsaturated heterocyclyl group;
and/or enantiomers and/or racemates and/or diastereomers and/or pharmaceutically acceptable salts thereof formed with acids or bases

a.) Converting a compound of formula (VI)

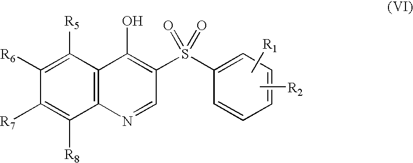

wherein R₁, R₂, R₅, R₆, R₇ and R₈ are as defined above for a compound of formula (I), to a compound of formula (VII)

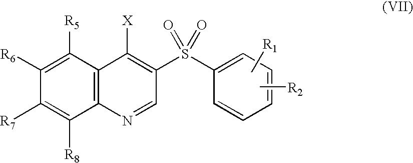

wherein X is selected from halogen, benzenesulfonyloxy or trifluoromethanesulfonyloxy groups and R₁, R₂, R₅, R₆, R₇ and R₈ are as defined above for a compound of formula (I), thereafter reacting the obtained compound of formula (VII) with a compound of formula (VIII):

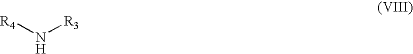

wherein R₃ and R₄ are as defined above for a compound of formula (I) to give a compound of formula (I), and optionally forming enantiomers and/or racemates and/or diastereomers and/or pharmaceutically acceptable salts thereof, or

b.) Reacting a compound of formula (XV):

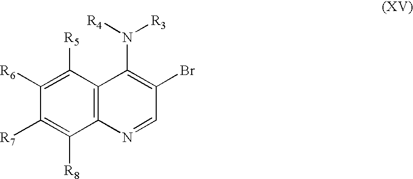

wherein R₃, R₄, R₅, R₆, R₇ and R₈ are as defined above for a compound of formula (I), with a compound of formula (III):

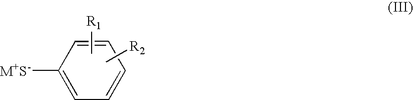

wherein M is selected from alkali or alkaline-earth metals, R₁ and R₂ are as defined above for a compound of formula (I), to give a compound of formula (XVI):

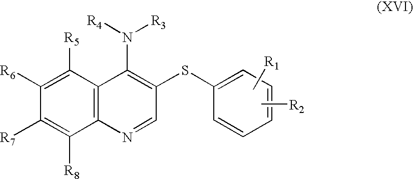

wherein R₁, R₂, R₃, R₄, R₅, R₆, R₇ and R₈ are as defined above for a compound of formula (I), thereafter oxidizing a compound of formula (XVI) to obtain a compound of formula (XVII):

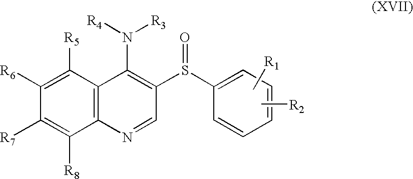

wherein R₁, R₂, R₃, R₄, R₅, R₆, R₇ and R₈ are as defined above for a compound of formula (I), thereafter oxidizing a compound of formula (XVII) to obtain a compound of formula (I), and optionally forming enantiomers and/or racemates and/or diastereomers and/or pharmaceutically acceptable salts thereof or

c.) interconverting one compound of formula (I), wherein the meaning of R₁, R₂, R₃, R₄, R₅, R₆, R₇ and R₈ is as defined above for the formula (I) to a different compound of formula (I), wherein the meaning of R₁, R₂, R₃, R₄, R₅, R₆, R₇ and R₈ is as described above for the formula (I);

where appropriate, separating the enantiomers and/or racemates and/or diastereomers of compounds of formula (I), wherein the meaning of R₁, R₂, R₃, R₄, R₅, R₆, R₇ and R₈ is as described above for the formula (I) by conventional methods;
and optionally thereafter forming salts and/or hydrates and/or solvates of compounds of formula (I).

A process for the preparation of a compound of formula (VI)

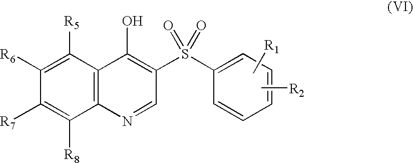

wherein
R₁ and R₂ are independently selected from the group consisting of hydrogen, halogen, alkyl, alkoxy, cyano, optionally substituted amino group or saturated heterocyclyl group, wherein the heteroatom is N;
R₅, R₆, R₇ and R₈ are independently selected from the group consisting of hydrogen, halogen, alkyl, alkoxy, cyano group, or
R₆ and R₇ together with the atoms to which they are attached can form an unsaturated heterocyclyl group;
and/or enantiomers and/or racemates and/or diastereomers and/or pharmaceutically acceptable salts thereof formed with acids or bases.

a.) reacting a compound of formula (II):

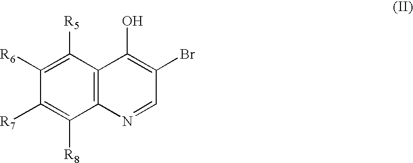

wherein R₅, R₆, R₇ and R₈ are as defined above for a compound of formula (I), with a compound of formula (III):

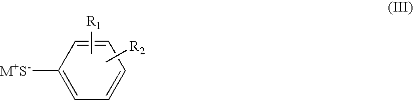

wherein M is selected from alkali metals or alkaline-earth metals, R₁ and R₂ are as defined above for a compound of formula (I), to give a compound of formula (IV):

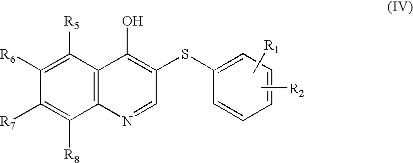

wherein R₁, R₂, R₅, R₆, R₇ and R₈ are as defined above for a compound of formula (I), thereafter oxidizing a compound of formula (IV) to obtain a compound of formula (V):

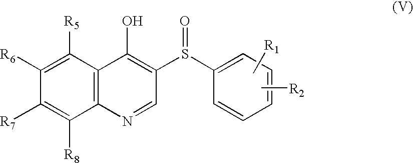

wherein R₁, R₂, R₅, R₆, R₇ and R₈ are as defined above for a compound of formula (I), thereafter oxidizing a compound of formula (V) to obtain a compound of formula (VI), and optionally forming enantiomers and/or racemates and/or diastereomers and/or pharmaceutically acceptable salts thereof or

b.) reacting a compound of formula (IX):

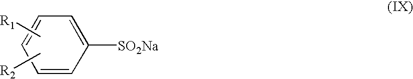

wherein R₁ and R₂ are as defined above for a compound of formula (I), with α-halogen-acetic acid esters of formula (X):

Hlg-CH₂—COOR₉ (X)

wherein Hlg is halogen and R₉ is an ethyl or methyl group, to obtain a compound of formula (XI):

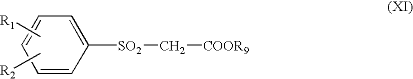

wherein R₁ and R₂ are as defined above for a compound of formula (I) and R₉ is as defined above for compounds of formula (X); reacting a compound of formula (XI) with a trialkyl orthoformate of formula (XII):

CH(OR₁₀)₃ (XII)

wherein R₁₀ is an ethyl or methyl group, to obtain a compound of formula (XIII):

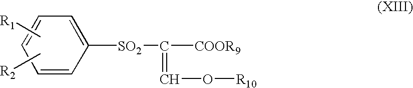

wherein R₁ and R₂ are as defined above for a compound of formula (I), R₉ is as defined above for compounds of formula (X) and R₁₀ is as defined above for compounds of formula (XII); reacting a compound of formula (XIII) with an aniline derivative of formula (XIV):

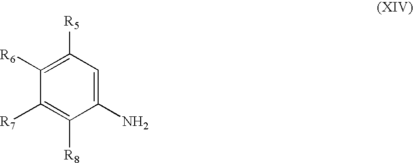

wherein R₅, R₆, R₇ and R₈ are as defined above for a compound of formula (I), to obtain a compound of formula (VI), and optionally forming enantiomers and/or racemates and/or diastereomers and/or pharmaceutically acceptable salts thereof.

According to Scheme 1a, 3-bromo-derivatives of formula (II) can be substituted by alkali or alkaline-earth metal salts (e.g. sodium salt) of thiophenols of formula (III) to provide a compound of formula (IV) (Bioorg. Med. Chem. Lett. 2001, 9, 1141-1144). Advantageously the substitution can be carried out by palladium catalyzation and under microwave conditions. To decrease the time of the reaction Pd-catalysis and/or microwave irradiation is required.

Oxidation of 3-arenesulfanyl-4-hydroxyquinolines of formula (IV) can be accomplished in a suitable acid (e.g.: trifluoroacetic acid) with hydrogen peroxid to give sulfoxides of formula (V) and sulfones of formula (VI), respectively.

Conversion of 4-hydroxyquinoline derivatives of formula (VI) to compounds of formula (VII) can be carried out by known halogenation methods with suitable reagents (e.g. POCl₃, SOCl₂, PCl₅, POBr₃, PBr₃) or by known acylation methods eg. with benzenenesulfonyl chloride or trifluoromethanesulfonis anhydride.

To obtain compounds of formula (VII) the reaction can also be accomplished by halogenation or acylation of 4-hydroxyquinoline derivatives of formula (V) and oxidation of the resulted compound by the known methods or according to the above mentioned methods.

Compounds of formula (I) can be prepared by the aromatic nucleophilic substitution reaction of compounds of formula (VII) with primary or secondary amines of formula (VIII).

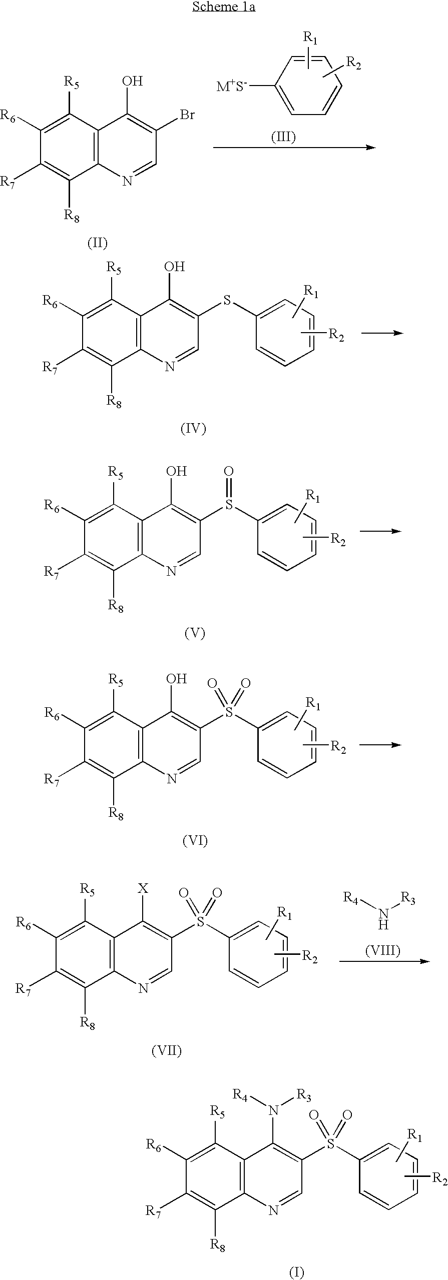

According to Scheme 1b another method to prepare intermediates of formula (I) via intermediates of formula (VI) is to react compounds of formula (IX) with α-halogen-acetic acid esters of formula (X) in a suitable solvent (e.g. DMF, water). Compounds of formula (IX) can be purchased or can be prepared from the appropriate benzenesulfonyl chloride derivatives by known methods (e.g. Org. Lett. 2003, 5(21), 3895-3898). Compounds of formula (XIII) can be prepared by the reaction of compounds of formula (XI) and compounds of formula (XII) in the presence of acetic anhydride [J. Org. Chem. USSR (Engl. Transl., 1980, 16(7), 1275-1278; Zh. Org. Khim, 1980, 16(7), 1483-1487]. Reaction of compounds of formula (XIII) with aniline derivatives of compounds of formula (XIV) gives benzenesulfonil-phenylamino-acrylic acid esters [e.g. J. Org. Chem. USSR (Engl. Transl., 1980, 16(7), 1275-1278; Zh. Org. Khim, 1980, 16(7), 1483-1487] that can be in situ converted into 4-hydroxy-quinoline derivatives of formula (VI) (see analogous reaction: J. Chem. Soc. Perkin Trans. 1, 1994, 4, 387-392). To obtain compounds of formula (I) the same preparation steps can be used as described above in scheme 1a.

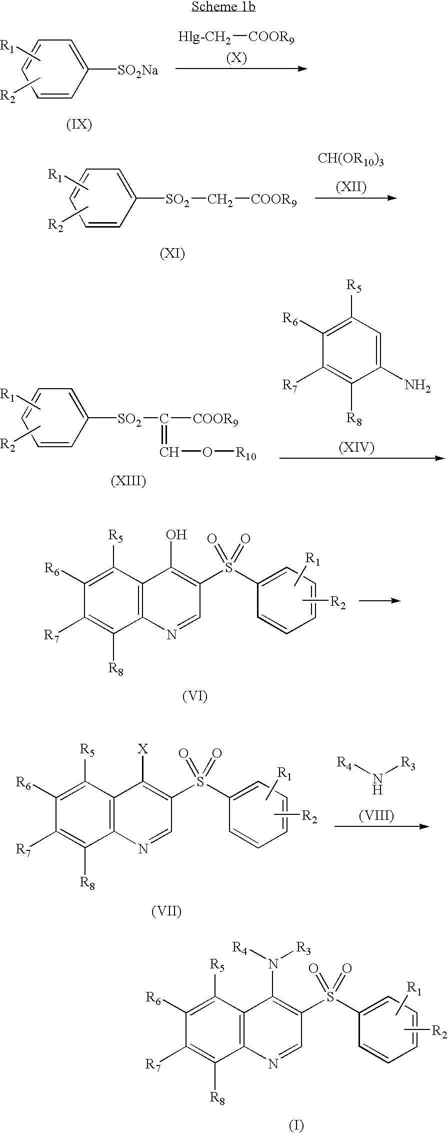

Compounds of formula (IV), compounds of formula (V), compounds of formula (VI) and compounds of formula (VII) and/or enantiomers and/or racemates and/or diastereomers and/or pharmaceutically acceptable salts thereof formed with acids or bases are new.

According to scheme 2 4-amino-3-bromoquinolines of formula (XV) can be prepared by known methods (J. Med. Chem. 2000, 43, 4667-4677). According to Scheme 2,4-amino-3-bromoquinolines of formula (XV) can be substituted by e.g. sodium salts of thiophenols of formula (III) to provide compounds of formula (XVI). The aromatic nucleophilic reaction can be carried out advantageously by palladium catalyst and/or under microwave conditions.

Oxidation of 4-amino-3-arylsulfanylquinolines of formula (XVI) can be accomplished by known methods, preferably in a suitable acid (e.g. acetic acid) at 0-5° C. with potassium permanganate to give 4-amion-3-arylsulfinylquinolines of formula (XVII) or with aqueous hydrogen peroxid in a suitable acid (e.g.: acetic acid or trifluoroacetic acid).

To obtain compounds of formula (I) a further oxidation of a compound of formula (XVII) is required. The reaction can be carried out by known methods, preferably in a suitable acid (e.g. acetic acid) at 0-5° C. with potassium permanganate.

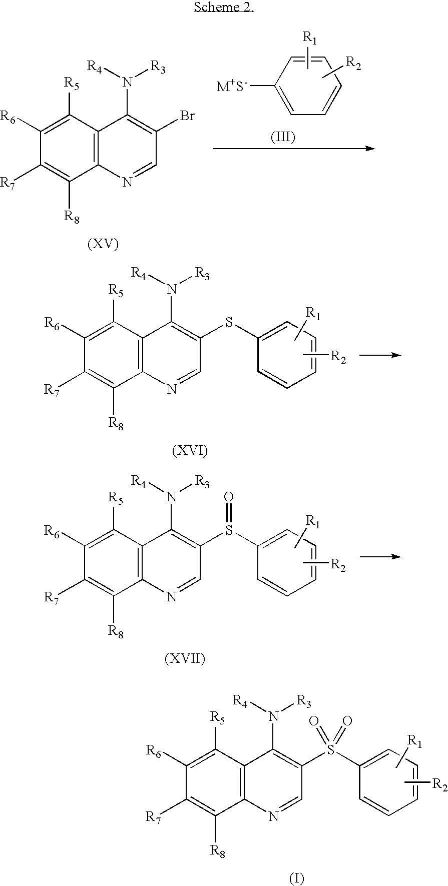

Compounds of formula (XVI) and compounds of formula (XVII) and/or enantiomers and/or racemates and/or diastereomers and/or pharmaceutically acceptable salts thereof formed with acids or bases are new.

Certain compounds of formula (I) can exist as enantiomers and racemates and diastereomers, too. These stereoisomers can optionally be separated, e.g. by chiral column chromatography or by crystallization (in the case of diastereomers).

The pure enantiomers and/or racemates and/or diastereomers of compounds of formula (I) can be prepared from stereochemically and/or geometrically pure precursors, too.

Compounds of formula (I) containing basic function(s) can be transformed into the salts thereof with acids and/or can be liberated from the obtained acid addition salts by treatment with a base.

Compounds of formula (I) can be transformed into hydrates and/or solvates.

The compounds of formula (I) can optionally be intercoverted to a different compound of formula (I) by conventional synthetic methods.

Biological Test Methods

mGluR5 Receptor Binding Test

The mGluR5 receptor binding was determined according to the modified method of Gasparini et. al. (Bioorg. Med. Chem. Lett., 2000, 12, 407). Based on the high homology between the human and rat mGluR5 receptors, rat cerebro-cortical membrane preparation was used to determine the binding characteristics of the reference compounds and novel compounds to the rat mGluR5. The A18 cell line expressing hmGluR5a (purchased from Euroscreen) was used to determine binding characteristics of the chemical compounds to the human mGluR5a receptor. [³H]-M-MPEP (2 nM) was used as radioligand. The nonspecific binding was determined in the presence of 10 μM M-MPEP.

Assessment of Functional Activity

Cell Cultures for Native Rat mgluR5 Receptors

Functional potency at native rat mGluR5 receptors was estimated using primary neocortical cell cultures derived from 17 day old Charles River rat embryos (for the details on the preparation of neural cell cultures see Johnson, M. L; Bunge, R. P. (1992): Primary cell cultures of peripheral and central neurons and glia. In: Protocols for Neural Cell Culture, eds: Fedoroff, S.; Richardson A., The Humana Press Inc., 51-77). After isolation the cells were plated onto standard 96-well microplates and the cultures were maintained in an atmosphere of 95% air-5% CO₂ at 37° C. The neocortical cultures were used for the calcium measurements after 5-7 days in vitro.

Cell Cultures for Recombinant Human mgluR5a Receptors

Chinese hamster ovary (CHO) cells stably expressing recombinant human mGluR5a (CHO-mGluR5a, Euroscreen) receptors were cultured in F12 medium containing 10% FCS, 1% antibiotic antimycotic solution, 400 μg/ml G418, 250 μg/ml zeocin, 5 μg/ml puromycin. Cells were kept at 37° C. in a humidified incubator in an atmosphere of 5% CO₂/95% air and were passaged three times a week. Cells were plated at 2.5-3.5×104 cell/well on standard 96-well microplates, receptor expression was induced by adding 600 ng/ml doxycycline on the next day. The calcium measurements were carried out 16-24 hours after the addition of the inducing agent.

Fluorimetric Measurement of Cytosolic Calcium Concentration

Measurements of cytosolic calcium concentration ([Ca²⁺]ᵢ) were carried out on primary neocortical cell cultures and on CHO-mGluR5a cells stably expressing human mGluR5a receptors. Cells were grown in standard 96-well microplates and before the measurement were loaded with a fluorescent Ca²⁺-sensitive dye, fluo-4/AM (2 μM): the neural cultures were loaded in their growth medium, CHO-mGluR5a cells were loaded in assay buffer (145 mM NaCl, 5 mM KCl, 2 mM MgCl₂, 2 mM CaCl₂, 10 mM HEPES, 20 mM D-glucose, 2 mM probenecid, pH=7.4) supplemented with 2 mM Na-pyruvate and 30 μg/ml glutamate-pyruvate transaminase (in case of CHO-mGluR5a cells these supplements were also present during the course of the [Ca²⁺]ᵢ measurements). Loading was done by incubating the cells with 100 μl/well dye solution at 37° C. in a humidified incubator in an atmosphere of 5% CO₂/95% air for 40-120 min. To stop dye loading cells were washed twice with assay buffer. After washing, various concentrations of the test compounds (diluted in assay buffer from a DMSO or a dimethylformamide (DMF) stock solution, final DMSO/DMF concentration was <0.1%) or buffer were added to each well depending on the experimental setup. In the case of neocortical cultures the assay buffer also contained TTX (0.5 μM, to suppress spontaneous oscillations of [Ca²⁺]ᵢ.

After incubation at 37° C. for 10-20 minutes baseline and agonist-evoked changes of [Ca²⁺]ᵢ were measured column by column with a plate reader fluorimeter (FlexStation II, Molecular Devices). Excitation and detection of emission was carried out from the bottom of the plate. The whole measurement process was performed at 37° C. and was controlled by custom software. Inhibitory potency of the test compounds was assessed by measuring the reduction in the agonist-evoked [Ca²⁺]ᵢ-elevation in the presence of different concentrations of the compounds. DHPG was used as agonist for both cultures, the concentration was 20 μM for the neocortical cultures. In the case of CHO-mGluR5a cells DHPG was applied at an EC80 concentration, the EC80-values were derived from daily determined dose-response curves.

Fluorescence data were expressed as ΔF/F (fluorescence change normalized to baseline). All treatments on a single plate were measured in multiple wells. Data from all wells with the same treatment were averaged and the average values were used for analysis. Inhibitory potency of a compound at a single concentration point was expressed as percent inhibition of the control agonist response. Sigmoidal concentration-inhibition curves were fitted to the data (derived from at least three independent experiments) and IC₅₀-values were determined as the concentration that produces half of the maximal inhibition caused by the compound. Raw fluorescence data were analyzed using Soft Max Pro (Molecular Devices), curve fitting was done with GraphPad Prism.

Results

Compounds of formula (I) of the present invention showed affinity for both rat and human mGluR5 receptors and proved to be functional antagonists, that is they inhibited functional responses elicited by stimulation of mGluR5 receptors.

The invention is further illustrated by the following non-limiting examples.

Unless specifically stated otherwise, all operation were carried out at room temperature, that is at a temperature range of 18-25° C. The course of reactions was followed by thin layer chromatography (TLC) and reaction times are given for illustration only. The structure of all intermediates and end products were elucidated by IR, NMR and MS spectroscopy. When given yields are for illustration only. When given, NMR data are in the form of delta (δ) values for major diagnostic protons, given in parts per million (ppm) relative to tetramethylsilane (TMS) as internal standard, using the indicated solvent. Conventional abbreviations are used for signal shape.

### EXAMPLES

All starting materials are either commercially available or can be synthesized by different known methods described in the literature.

### Example 1

### 3-(3,4-Dimethyl-benzenesulfonyl)-4-(morpholin-4-yl)-quinoline

### Table I Compound 1

### 4-(Morpholin-4-yl)-quinoline

A mixture of morpholine (0.86 ml, 9.9 mmol) and 4-chloroquinoline (0.74 g, 4.5 mmol) was heated up to 195° C. in a microwave reactor during 60 seconds. The reaction mixture was cooled to 150° C. and it was kept at the same temperature for 10 minutes. The mixture was dissolved in dichloromethane (40 ml), extracted with water (5×40 ml) and the solvent was evaporated in vacuo to obtain the title compound.

Yield.: 94%.

¹H NMR (300 MHz, DMSO-d₆, 30° C.): 3.19-3.27 (m, 4H); 3.95-4.04 (m, 4H); 7.02 (d, 1H, J=5.0 Hz); 7.49 (ddd, 1H, J=8.4, 6.9, 1.3 Hz); 7.67 (ddd, 1H, J=8.4, 6.9, 1.5 Hz); 8.03 (ddm, 1H, J=8.4, 1.5 Hz); 8.07 (ddm, 1H, J=8.4, 1.3 Hz); 8.76 (d, 1H, J=5.0 Hz). MS (EI) M⁺=214.

### 3-Bromo-4-(morpholin-4-yl)-quinoline

4-(Morpholin-4-yl)-quinoline (2.8 g, 13 mmol) was dissolved in a mixture of acetic acid (26 mL) and dichloromethane (9 ml) at 0-5° C. A solution of bromine (0.7 ml, 13.6 mmol) in a mixture of dichloromethane (3.5 ml) and acetic acid (7.0 ml) was added dropwise for 3 hours. The reaction mixture was stirred for 2 hours at 0-5° C. The residue was dissolved in ethyl acetate (200 ml), extracted with sodium carbonate (170 ml, 5% m/m) and dried over anhydrous sodium sulfate. The solvent was evaporated, and the crude product (3.5 g) was purified by gradient silica gel flash chromatography (300 g silica gel, eluent A: n-hexane:chloroform=3:7, eluent B:chloroform) to give the title compound.

Yield.: 74%.

¹H NMR (300 MHz, DMSO-d₆, 30° C.): 3.20-3.57 (brm, 4H); 3.80-3.91 (m, 4H); 7.65 (ddd, 1H, J=8.4, 6.9, 1.4 Hz); 7.79 (ddd, 1H, J=8.4, 6.9, 1.4 Hz); 8.01 (ddm, 1H, J=8.4, 1.4 Hz); 8.29 (ddm, 1H, J=8.4, 1.4 Hz); 8.84 (s, 1H). MS (EI): M⁺=292 (⁷⁹Br)

### 3-(3,4-Dimethyl-benzenesulfanyl)-4-(morpholin-4-yl)-quinoline hydrochloride

A mixture of 3-bromo-4-(morpholin-4-yl)-quinoline (293 mg, 1 mmol), 3,4-dimethylthiophenol (0.27 ml, 1.95 mmol), sodium-tert-butylate (187 mg, 1.95 mmol), tetrakis-(triphenylphosphin)palladium (230 mg, 0.2 mmol) and tert-butanol (0.7 ml) or DMF (0.7 ml) was heated in a microwave reactor at 143° C. for 2 hours. The solvent was evaporated in vacuo and the residue was dissolved in dichloromethane, extracted with water, dried over anhydrous sodium sulphate and the solvent was removed in vacuo. The residue was purified by gradient flash chromatography (40 g silicagele, eluent A: n-hexane:chloroform=3:7, eluent B:chloroform, flow rate: 40 ml/min) to give the crude product. The crude product was dissolved in ethyl acetate (5 ml) and a solution of hydrogen chloride in ethyl acetate (0.5 ml, c=1.6 M, 0.8 mmol) was added dropwise. The precipitate was filtered off, washed with ethyl acetate and dried in vacuo to give the title compound.

Yield.: 64%.

¹H NMR (300 MHz, DMSO-d₆, 30° C.): 2.18 (s, 3H); 2.19 (s, 3H); 3.70-3.87 (m, 8H); 6.98 (dd, 1H, J=7.9, 2.1 Hz); 7.09-7.17 (m, 2H); 7.78 (ddd, 1H, J=8.6, 7.0, 1.1 Hz); 8.01 (ddd, 1H, J=8.5, 7.0, 1.1 Hz); 8.23 (dd, 1H, J=8.5, 1.1 Hz); 8.30 (dd, 1H, J=8.6, 1.1 Hz); 8.74 (s, 1H).

MS (EI): M⁺=350

### 3-(3,4-Dimethyl-benzenesulfinyl)-4-(morpholin-4-yl)-quinoline hydrochloride

3-(3,4-Dimethyl-benzenesulfanyl)-4-(morpholin-4-yl)-quinoline hydrochloride (280 mg, 0.8 mmol) was dissolved in a mixture of acetic acid (6 ml) and water (22 ml) at 0-5° C. To the mixture a solution of potassium permanganate in water (c=0.1 M, 1.65 ml, 0.165 mmol) was added dropwise for 3 hours at 0-5° C. The reaction mixture was stirred for 1 hour at 0-5° C. and water (30 ml), dichloromethane (30 ml) and potassium carbonate (≅10 g) was added to the solution. The aqueous phase was extracted with dichloromethane (2×40 ml) and the organic layer was dried over anhydrous sodium sulfate. The solvent was evaporated and the crude product was purified by gradient flash chromatography (100 g silicagele, eluent A: n-hexane:chloroform=3:7, eluent B:chloroform). The purified product was dissolved in ethyl acetate (1 ml) and a solution of hydrogen chloride in ethyl acetate (0.4 ml, c=1.6 M, 0.64 mmol) was added. The precipitate was filtered off, washed with ethyl acetate and dried in vacuo to give the title compound.

Yield.: 68%.

¹H NMR (300 MHz, DMSO-d₆, 30° C.): 2.26 (s, 6H); 3.31-3.44 (m, 2H); 3.61-3.72 (m, 2H); 3.72-3.83 (m, 2H); 3.84-3.95 (m, 2H); 7.34 (d, 1H, J=7.8 Hz); 7.43 (dd, 1H, J=7.8, 2.1 Hz); 7.54 (d, 1H, J=2.1 Hz); 7.78 (ddd, 1H, J=8.6, 7.0, 1.3 Hz); 8.02 (ddd, 1H, J=8.5, 7.0, 1.3 Hz); 8.17-8.28 (m, 2H); 9.06 (s, 1H).

MS (EI): M⁺=366.

### 3-(3,4-Dimethyl-benzenesulfonyl)-4-(morpholin-4-yl)-quinoline

### Table I Compound 1

3-(3,4-Dimethyl-benzenesulfinyl)-4-(morpholin-4-yl)-quinoline hydrochloride (322 mg, 0.8 mmol) was dissolved in a mixture of acetic acid (7 ml) and water (25 ml) at 0-5° C. To the mixture a solution of potassium permanganate in water (c=0.1 M, 4 ml. 4 mmol) was added dropwise for 4 hours at 0-5° C. The reaction mixture was stirred for 1 hour at 0-5° C., water (30 ml), dichlioromethane (30 ml) and potassium carbonate (≅10 g) was added to the solution. The aqueous phase was extracted with dichloromethane (2×40 ml) and the organic layer was dried over anhydrous sodium sulfate. The solvent was evaporated and the crude product was purified by gradient flash chromatography (100 g silicagele, eluent A: n-hexane:chloroform=3:7, eluent B:chloroform) to give the title compound.

Yield.: 38%.

¹H NMR (500 MHz, DMSO-d₆, 30° C.): 2.28 (s, 3H); 2.30 (s, 3H); 3.05-3.13 (m, 4H); 3.45-3.52 (m, 4H); 7.39 (d, 1H, J=7.9 Hz); 7.64 (dd, 1H, J=7.9, 1.9 Hz); 7.67 (d, 1H, J=1.9 Hz); 7.74 (ddd, 1H, J=8.6, 6.9, 1.2 Hz); 7.96 (ddd, 1H, J=8.5, 6.9, 1.2 Hz); 8.20 (dd, 1H, J=8.5, 1.2 Hz); 8.32 (dd, 1H, J=8.6, 1.2 Hz); 9.39 (s, 1H).

MS (FAB): [M+H]⁺=383

### 3-(3,4-Dimethyl-benzenesulfonyl)-4-(morpholin-4-yl)-quinoline hydrochloride

3-(3,4-Dimethyl-benzenesulfonyl)-4-(morpholin-4-yl)-quinoline (80 mg, 0.21 mmol) was dissolved in ethyl acetate (3 ml) and a solution of hydrogen chloride in ethyl acetate (c=1.6 M, 0.28 ml, 0.45 mmol) was added dropwise to the solution. The solid was filtered off, washed with ethyl acetate and dried in vacuo to give the title compound.

Yield.: 86%.

¹H NMR (300 MHz, DMSO-d₆, 30° C.): 2.29 (s, 3H); 2.30 (s, 3H); 3.13-3.23 (m, 4H); 3.48-3.57 (m, 4H); 7.40 (d, 1H, J=8.0 Hz); 7.67 (dd, 1H, J=8.0, 2.1 Hz); 7.70 (d, 1H, J=2.1 Hz); 7.77 (ddd, 1H, J=8.6, 6.9, 1.3 Hz); 7.99 (ddd, 1H, J=8.5, 6.9, 1.3 Hz); 8.22 (dd, 1H, J=8.5, 1.3 Hz); 8.33 (dd, 1H, J=8.6, 1.3 Hz); 9.39 (s, 1H).

MS (FAB): [M+H]⁺=383

### Example 2

### 3-(4-Methyl-benzenesulfonyl)-4-(4-methyl-piperidin-1-yl)-quinoline

### Table I Compound 2

### 3-(4-Methyl benzenesulfanyl)-4-hydroxy-quinoline

A mixture of 3-bromo-4-hydroxyquinoline (0.448 g, 2 mmol; J. Am. Chem. Soc. 1946, 68, 1229-1231), 4-methylbenzenethiol (0.30 g, 2.4 mmol), tetrakis-(triphenylphosphin)palladium (0.115 g, 0.1 mmol), sodium-tert-butylate (0.23 g, 2.4 mmol) and DMF (2.0 ml) was stirred and irradiated at 142° C. for 3 hours in a 8-ml microwave vial. The solvent was evaporated in vacuo and the residue was purified by gradient silica gel flash chromatography (80 g silica gel, eluent A: CHCl3, eluent B: CHCl3:MeOH=95:5) to give the title compound.

Yield: 61%.

¹H NMR (500 MHz, DMSO-d₆, 25° C.): 2.23 (s, 3H); 7.04-7.09 (m, 4H); 7.39 (ddd, 1H, J=8.1, 7.0, 1.1 Hz); 7.61 (ddm, 1H, J=8.4, 1.1 Hz); 7.70 (ddd, 1H, J=8.4, 7.0, 1.5 Hz); 8.12 (ddm, 1H, J=8.1, 1.5 Hz); 8.30 (s, 1H); 12.23 (brs, 1H).

M⁺(EI)=267

### 3-(4-Methyl-benzenesulfonyl)-4-quinoline

3-(4-Methyl-benzenesulfanyl)-4-hydroxyquinoline (0.25 g, 0.936 mmol) was dissolved in trifluoroacetic acid (5.0 ml) and a solution of hydrogen peroxide in trifluoroacetic acid (c=3.0M, 3.7 ml) was added dropwise for 8 hours at room temperature. 6 ml water was added dropwise to the reaction mixture. The precipitate was filtered off, washed with water and dried in vacuo to give the title compound.

Yield: 87%.

¹H NMR (500 MHz, DMSO-d₆, 25° C.): 2.37 (s, 3H); 7.34-7.40 (m, 2H); 7.44 (ddd, 1H, J=8.1, 7.0, 1.2 Hz); 7.69 (ddm, 1H, J=8.4, 1.2 Hz); 7.76 (ddd, 1H, J=8.4, 7.0, 1.5 Hz); 7.88-7.95 (m, 2H); 8.05 (ddm, 1H, J=8.1, 1.5 Hz); 8.74 (d, 1H, J=6.8 Hz); 12.75 (d, 1H, J=6.8 Hz).

MS (FAB): [M+H]⁺=300

### 4-Chloro-3-(4-methyl-benzenesulfonyl)-quinoline

A mixture of 3-(4-methyl-benzenesulfonyl)-4-hydroxyquinoline (0.24 g, 0.8 mmol) and phosphoryl chloride (15 ml) was stirred at 135° C. for 5 hours. Phosphoryl chloride was distilled off, and the residue was poured onto ice. The slurry was stirred for 2 hours at 0-5° C., neutralized with sodium carbonate and extracted with chloroform (50 ml). The organic layer was dried over anhydrous sodium sulphate, filtered and the solvent was removed in vacuo to give the title compound.

Yield: 91%.

¹H NMR (500 MHz, CDCl₃, 25° C.): 2.42 (s, 3H); 7.31-7.36 (m, 2H); 7.72 (ddd, 1H, J=8.6, 6.9, 1.1 Hz); 7.91 (ddd, 1H, J=8.5, 6.9, 1.4 Hz); 7.92-7.96 (m, 2H); 8.21 (dm, 1H, J=8.5 Hz); 8.33 (ddm, 1H, J=8.6, 1.4 Hz); 9.63 (s, 1H).

MS (EI) [M+H]⁺=285 (³⁵Cl)

### 3-(4-Methyl-benzenesulfonyl)-4-(4-methyl-piperidin-1-yl)quinoline

### Table I Compound 2

4-Chloro-3-(4-methyl-benzenesulfonyl)-quinoline (0.25 mmol, 79 mg) was dissolved in acetonitrile (0.5 ml) and 4-methylpiperidine (63 μL, 0.55 mmol) was added. The reaction mixture was stirred at 50° C. for three hours then it was stirred at room temperature overnight. The solvent was evaporated in vacuo and the residue was purified by flash chromatography (1.0 g silica gel, chloroform).

Yield: 87%.

¹H NMR (500 MHz, DMSO-d₆, 25° C.): 0.92 (d, 3H, J=6.6 Hz); 0.92-1.04 (m, 2H); 1.38-1.47 (m, 2H); 1.47-1.61 (m, 1H); 2.39 (s, 3H); 2.83-2.93 (m, 2H); 3.26-3.34 (m, 2H); 7.39-7.45 (m, 2H); 7.71 (ddd, 1H, J=8.6, 7.0, 1.2 Hz); 7.72-7.78 (m, 2H); 7.93 (ddd, 1H, J=8.4, 7.0, 1.2 Hz); 8.16 (dd, 1H, J=8.4, 1.2 Hz); 8.28 (dd, 1H, J=8.6, 1.2 Hz); 9.35 (s, 1H).

MS (EI): M⁺=380

### 3-(4-Methyl-benzenesulfonyl)-4-(4-methyl-piperidin-1-yl)-quinoline hydrochloride

3-(4-Methyl-benzenesulfonyl)-4-(4-methyl-piperidin-1-yl)-quinoline (40 mg, 0.105 mmol) was dissolved in ethyl acetate (0.5 ml) then a solution of hydrogen chloride in ethyl acetate (c=1.6 M, 65 μl 0.105 mmol) was added dropwise to the solution. The solvent was removed in vacou to give the title compound.

Yield: 100%.

¹H NMR (500 MHz, DMSO-d₆, 25° C.): 0.93 (d, 3H, J=6.5 Hz); 1.08-1.20 (m, 2H); 1.48-1.57 (m, 2H); 1.57-1.68 (m, 1H); 2.41 (s, 3H); 3.19-3.29 (m, 2H); 3.32-3.42 (m, 2H); 7.42-7.49 (m, 2H); 7.77 (ddd, 1H, J=8.6, 7.0, 1.2 Hz); 7.81-7.86 (m, 2H); 8.01 (ddd, 1H, J=8.5, 7.0, 1.2 Hz); 8.19 (dd, 1H, J=8.5, 1.2 Hz); 8.29 (dd, 1H, J=8.6, 1.2 Hz); 9.37 (s, 1H).

MS (EI) M⁺=380.

### Example 3

### 3-(3,4-Dimethyl-benzenesulfonyl)-4-(4-methyl-piperidin-1-yl)-quinoline

### Table I Compound 3

### 3-(3,4-Dimethyl-benzenesulfanyl)-4-hydroxyquinoline

The title compound was prepared from 3-bromo-4-hydroxyquinoline and 3,4-dimethylthiophenol according to the method described in Example 2 for the preparation of 3-(4-Methyl-benzenesulfanyl)-4-hydroxyquinoline.

Yield: 58%.

¹H NMR (500 MHz, DMSO-d₆, 25° C.): 2.14 (s, 3H); 2.15 (s, 3H); 6.90 (dd, 1H, J=7.8, 2.0 Hz); 6.98-7.04 (m, 2H); 7.38 (ddd, 1H, J=8.1, 7.0, 1.2 Hz); 7.60 (ddm, 1H, J=8.4, 1.2 Hz); 7.69 (ddd, 1H, J=8.4, 7.0, 1.5 Hz); 8.11 (ddm, 1H, J=8.1, 1.5 Hz); 8.25 (s, 1H); 12.18 (brs, 1H).

MS (EI) M⁺=281.

### 3-(3,4-Dimethyl-benzenesulfonyl)-4-hydroxyquinoline

The title compound was prepared according to the method described in Example 2 for the preparation of 3-(4-Methyl-benzenesulfonyl)-4-hydroxyquinoline.

Yield: 89%.

¹H NMR (500 MHz, DMSO-d₆, 25° C.): 2.30 (s, 3H); 2.32 (s, 3H); 7.27 (d, 1H, J=8.5 Hz); 7.39 (ddd, 1H, J=8.1, 7.0, 1.2 Hz); 7.62 (ddm, 1H, J=8.4, 1.2 Hz); 7.68 (ddd, 1H, J=8.4, 7.0, 1.5 Hz); 7.78-7.82 (m, 2H); 8.15 (ddm, 1H, J=8.1, 1.5 Hz); 8.66 (s, 1H); 12.63 (brs, 1H).

MS (FAB): [M+H]⁺=314

### 4-Chloro-3-(3,4-dimethylbenzenesulfonyl)-quinoline

The title compound was prepared according to the method described in Example 2 for the preparation of 4-Chloro-3-(4-methylbenzenesulfonyl)-quinoline.

Yield: 92%.

¹H NMR (500 MHz, CDCl₃, 25° C.): 2.32 (s, 3H); 2.33 (s, 3H); 7.32 (d, 1H, J=8.1 Hz); 7.77 (d, 1H, J=2.1 Hz); 7.82 (dd, 1H, J=8.1, 2.1 Hz); 7.85 (ddd, 1H, J=8.6, 7.0, 0.9 Hz); 8.05 (ddd, 1H, J=8.5, 7.0, 1.3 Hz); 8.39-8.46 (m, 2H); 9.66 (s, 1H).

MS (EI) M⁺=331 (³⁵Cl)

### 3-(3,4-Dimethylbenzenesulfonyl)-4-(4-methyl-piperidin-1-yl)-quinoline

The title compound was prepared according to the method described in Example 2 for the preparation of compound 2 in Table I.

Yield: 87%.

¹H NMR (500 MHz, DMSO-d₆, 25° C.): 0.91 (d, 3H, J=6.5 Hz); 0.92-1.03 (m, 2H); 1.38-1.46 (m, 2H); 1.47-1.61 (m, 1H); 2.29 (s, 3H); 2.30 (s, 3H); 2.82-2.91 (m, 2H); 3.28-3.36 (m, 2H); 7.36 (d, 1H, J=8.1 Hz); 7.54 (dd, 1H, J=8.1, 2.0 Hz); 7.67 (d, 1H, J=2.0 Hz); 7.70 (ddd, 1H, J=8.5, 6.9, 1.2 Hz); 7.93 (ddd, 1H, J=8.5, 6.9, 1.2 Hz); 8.16 (dd, 1H, J=8.5, 1.2 Hz); 8.27 (dd, 1H, J=8.5, 1.2 Hz); 9.35 (s, 1H).

MS (EI) M⁺=394.

### 3-(3,4-Dimethyl-benzenesulfonyl)-4-(4-methyl-piperidin-1-yl)-quinoline hydrochloride

The title compound was prepared according to the method described in Example 2 for the preparation of the hydrochloride salt of compound 2 in Table I.

Yield: 100%.

¹H NMR (500 MHz, DMSO-d₆, 25° C.): 0.93 (d, 3H, J=6.5 Hz); 1.03-1.16 (m, 2H); 1.46-1.55 (m, 2H); 1.55-1.67 (m, 1H); 2.30 (s, 3H); 2.31 (s, 3H); 3.10-3.20 (m, 2H); 3.32-3.42 (m, 2H); 7.39 (d, 1H, J=8.1 Hz); 7.61 (dd, 1H, J=8.1, 2.0 Hz); 7.72 (d, 1H, J=2.0 Hz); 7.75 (ddd, 1H, J=8.6, 7.0, 1.2 Hz); 7.99 (ddd, 1H, J=8.6, 7.0, 1.2 Hz); 8.18 (dd, 1H, J=8.6, 1.2 Hz); 8.28 (dd, 1H, J=8.6, 1.2 Hz); 9.36 (s, 1H).

MS (EI) M⁺=394.

### Example 4

### 3-(4-Methyl-benzenesulfonyl)-4-(3-methyl-piperidin-1-yl)-quinoline

### Table I Compound 4

The title compound was prepared from 4-chloro-3-(4-methyl-benzenesulfonyl)-quinoline according to the method described in Example 2 for the preparation of compound 2 in Table I.

Yield: 90%.

¹H NMR (500 MHz, DMSO-d₆, 25° C.): 0.67 (d, 3H, J=6.7 Hz); 0.99-1.11 (m, 1H); 1.28-1.41 (m, 2H); 1.41-1.52 (m, 1H); 1.63-1.74 (m, 1H); 2.39 (s, 3H); 2.70-2.79 (m, 1H); 2.81-2.96 (m, 2H); 3.20-3.29 (m, 1H); 7.39-7.44 (m, 2H); 7.71 (ddd, 1H, J=8.5, 6.9, 1.2 Hz); 7.72-7.77 (m, 2H); 7.93 (ddd, 1H, J=8.4, 6.9, 1.2 Hz); 8.16 (dd, 1H, J=8.4, 1.2 Hz); 8.28 (dd, 1H, J=8.5, 1.2 Hz); 9.36 (s, 1H).

MS (EI) M⁺=380.

### Example 5

### 7-Chloro-3-(4-chloro-benzenesulfonyl)-4-(4-methyl-piperidin-1-yl)-quinoline

### Table I Compound 195

### (4-Chloro-benzenesulfonyl)-acetic acid methyl ester

The mixture of methyl bromoacetate (11.25 ml, 116 mmol) sodium 4-chloro-benzenesulfinate (25.2 g, 116 mmol) in DMF (120 ml) was stirred and heated at 80° C. for 2 h. The solution was diluted with water (360 ml). The separated oil was extracted with chloroform (200 ml) and washed with water (3×80 ml). The organic phase was evaporated in vacuo. The yield was 22.4 g (77.7%).

MS (EI) M⁺=249.

Applying the above procedure the following compounds were prepared: e.g.: (3-chloro-benzenesulfonyl)-acetic acid methyl ester (MS (EI) M⁺=249); (3,4-dichloro-benzenesulfonyl)-acetic acid methyl ester (MS (EI) M⁺=284); (4-methoxy-benzenesulfonyl)-acetic acid methyl ester (MS (EI) M⁺=245); (3-chloro-4-fluoro-benzenesulfonyl)-acetic acid methyl ester (MS (EI) M⁺=267).

### 2-(4-Chloro-benzenesulfonyl)-3-ethoxy-acrylic acid methyl ester

The mixture of (4-Chloro-benzenesulfonyl)-acetic acid methyl ester (22.4 g, 90 mmol), triethyl orthoformate (33.2 ml, 216 mmol) and acetic anhydride (19.1 ml, 203 mmol) was refluxed for 3 h with simultaneous distillation of ethanol, triethyl orthoformate and acetic anhydride, and then evaporated to dryness. The crude material (22.7 g, 82.8%) was used in the next step without purification.

MS (EI) M⁺−305.

Applying the above procedure the following compounds were prepared: e.g.: 2-(3-chloro-benzenesulfonyl)-3-ethoxy-acrylic acid methyl ester (MS (EI) M⁺=305); 2-(3-chloro-4-fluoro-benzenesulfonyl)-3-ethoxy-acrylic acid methyl ester (MS (EI) M⁺=323); 2-(3,4-dichloro-benzenesulfonyl)-3-ethoxy-acrylic acid methyl ester (MS (EI) M⁺=340); 2-(4-fluoro-benzenesulfonyl)-3-ethoxy-acrylic acid methyl ester (MS (EI) M⁺=289).

### 7-Chloro-3-(4-chloro-benzenesulfonyl)-4-hydroxyquinoline

The mixture of 2-(4-Chloro-benzenesulfonyl)-3-ethoxy-acrylic acid methyl ester (7.62 g, 25 mmol) and 3-chloroaniline (3.19 g, 25 mmol) in diphenyl ether (20 ml) was heated at near reflux for 1 h. After cooling the precipitate was filtered and washed with ether. The yield was 4.25 g (48.0%).

MS (EI) M⁺=355.

Applying the above procedure the following compounds were prepared: e.g.: 8-chloro-3-(4-chloro-benzenesulfonyl)-4-hydroxyquinoline (MS (EI) M⁺=355); 6-chloro-3-(4-fluoro-benzenesulfonyl)-4-hydroxyquinoline (MS (EI) M⁺=338); 6-cyano-3-(4-fluoro-benzenesulfonyl)-4-hydroxyquinoline (MS (EI) M⁺=329); 8-chloro-3-(3,4-dichloro-benzenesulfonyl)-4-hydroxyquinoline (MS (EI) M⁺=390); 8-ethyl-3-(4-chloro-benzenesulfonyl)-4-hydroxyquinoline (MS (EI) M⁺=348).

### 3-(4-chloro-benzenesulfonyl)-4,7-dichloroquinoline

7-Chloro-3-(4-chloro-benzenesulfonyl)-4-hydroxyquinoline (4.25 g, 12 mmol) in phosphorus(III) oxychloride (5.6 ml, 60 mmol) was refluxed for 3 h. The reaction mixture was poured into water (50 ml) and was alkalized with 5M sodium hydroxyde solution. After cooling the precipitate was filtered and washed with water. The yield was 3.8 g (85.0%).

MS (EI) M⁺=373.

Applying the above procedure the following compounds were prepared: e.g.: 3-(3-chloro-benzenesulfonyl)-4,6-dichloroquinoline (MS (EI) M⁺=373); 3-(4-chloro-benzenesulfonyl)-4,8-dichloroquinoline (MS (EI) M⁺=373); 4-chloro-6-cyano-3-(4-fluoro-benzenesulfonyl)-quinoline (MS (EI) M⁺=347); 4-chloro-3-(4-chloro-benzenesulfonyl)-6-fluoro-quinoline (MS (EI) M⁺=357).

### 7-Chloro-3-(4-chloro-benzenesulfonyl)-4-(4-methyl-piperidin-1-yl)-quinoline

### Table I Compound 195

### 3-(4-Chloro-benzenesulfonyl)-4,7-dichloroquinoline (186 mg, 0.5 mmol), 4-methylpiperidine (71 μl, 0.6 mmol) and DBU (90 μl, 0.6 mmol) in DMF (6 ml) were stirred for 1 h at 80° C. After cooling, water (30 ml) was added, The solid was filtered and crystallized from methanol. The yield was 167 mg (76.6%).

MS (EI) M⁺=436.

Examples of compounds of formula (I) and their affinity for rat mGluR5 receptors are given in the table below.

TABLE I

| Comp. | | | |
|---|---|---|---|
| No. | Structure | (M + H)⁺ | Kᵢ (nM) |
| 1 | 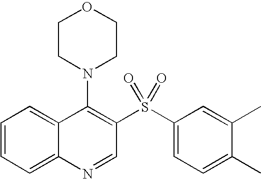 | 383 | *** |
| 2 | 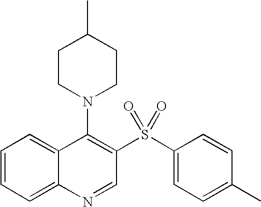 | 381 | *** |
| 3 | 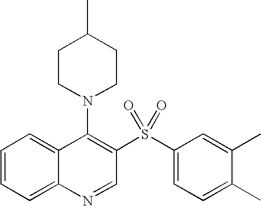 | 394 | *** |
| 4 | 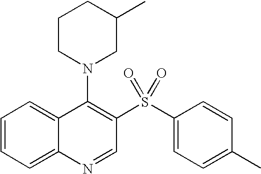 | 381 | *** |
| 5 | 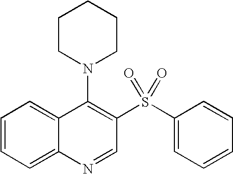 | 353 | ** |
| 6 | 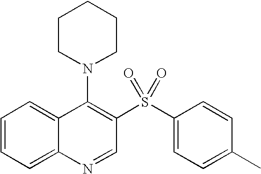 | 367 | *** |
| 7 | 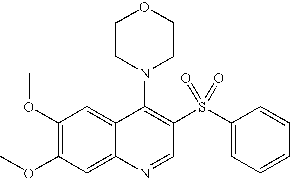 | 415 | * |
| 8 | 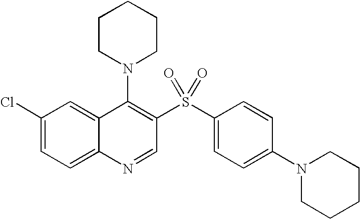 | 470 | * |
| 9 | 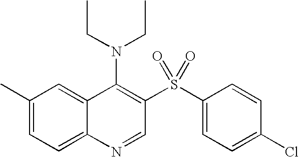 | 389 | *** |
| 10 | 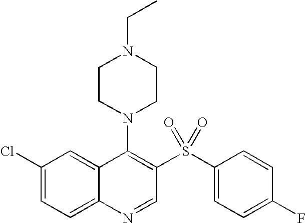 | 434 | * |
| 11 | 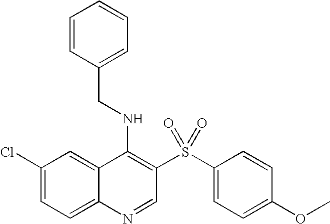 | 439 | * |
| 12 | 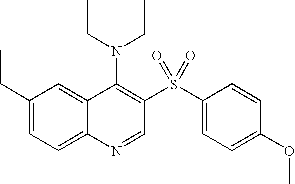 | 399 | * |
| 13 | 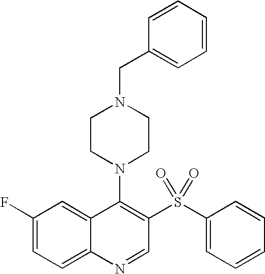 | 462 | * |
| 14 | 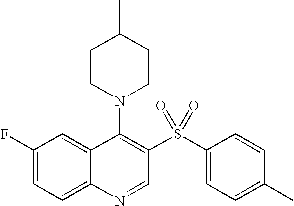 | 399 | ** |
| 15 | 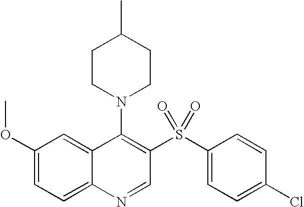 | 431 | * |
| 16 | 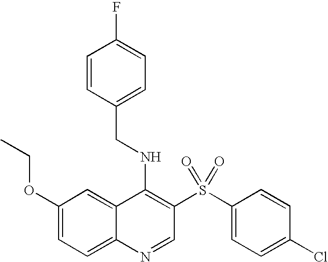 | 471 | *** |
| 17 | 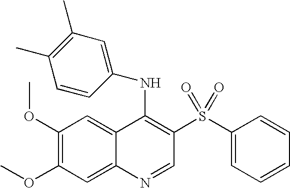 | 434 | * |
| 18 | 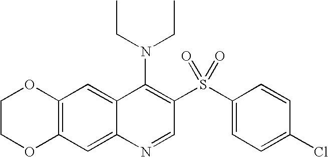 | 433 | * |
| 19 | 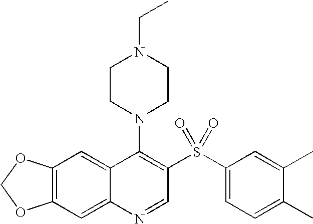 | 454 | * |
| 20 | 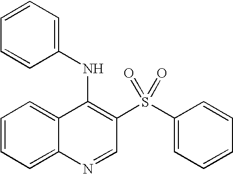 | 361 | * |
| 21 | 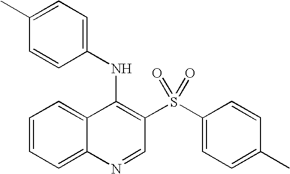 | 389 | * |
| 22 | 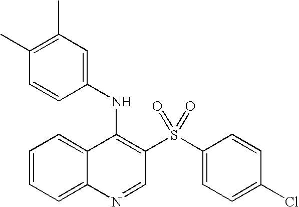 | 423 | * |
| 23 | 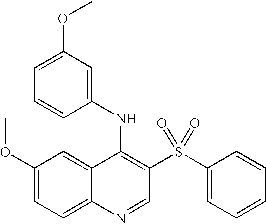 | 421 | * |
| 24 | 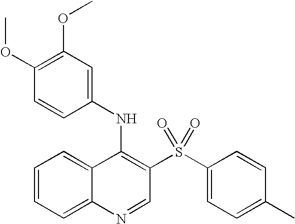 | 435 | * |
| 25 | 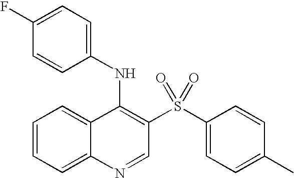 | 393 | * |
| 26 | 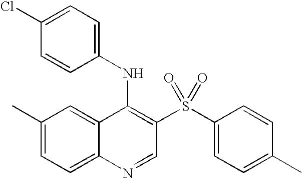 | 423 | * |
| 27 | 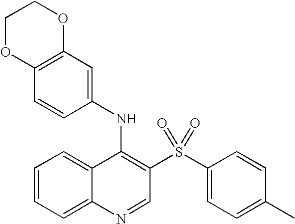 | 433 | * |
| 28 | 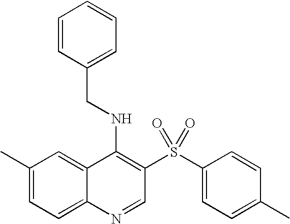 | 403 | * |
| 29 | 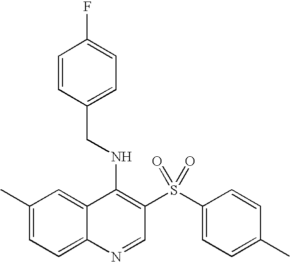 | 421 | * |
| 30 | 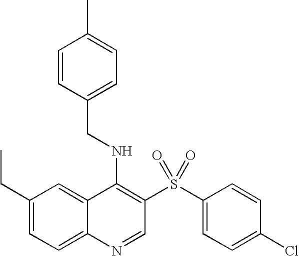 | 451 | * |
| 31 | 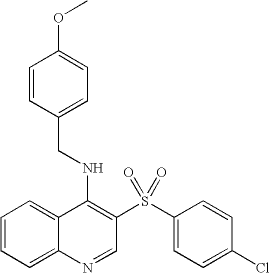 | 439 | * |
| 32 | 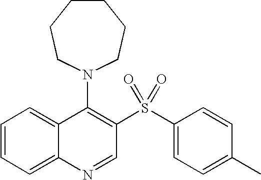 | 381 | *** |
| 33 | 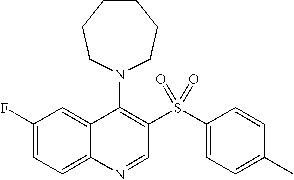 | 399 | * |
| 34 | 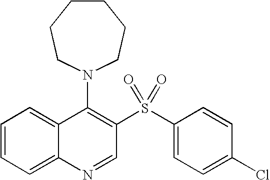 | 401 | ** |
| 35 | 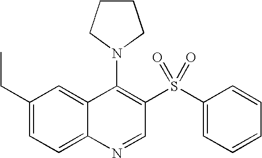 | 367 | * |
| 36 | 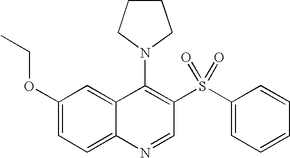 | 383 | * |
| 37 | 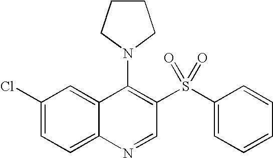 | 373 | * |
| 38 | 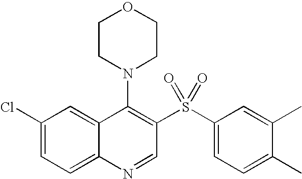 | 417 | * |
| 39 | 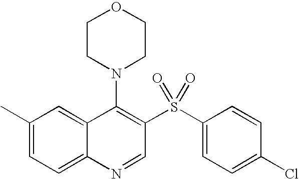 | 403 | * |
| 40 | 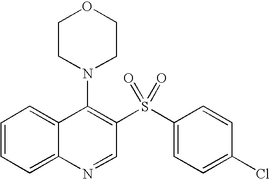 | 389 | * |
| 41 | 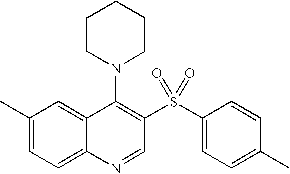 | 381 | * |
| 42 | 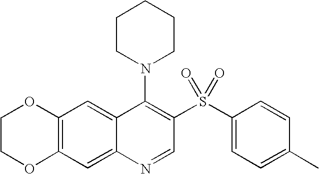 | 425 | * |
| 43 | 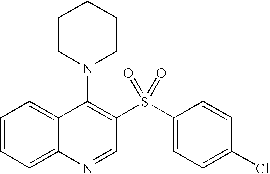 | 387 | * |
| 44 | 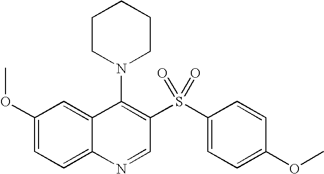 | 413 | * |
| 45 | 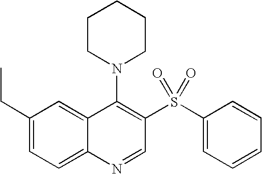 | 381 | * |
| 46 | 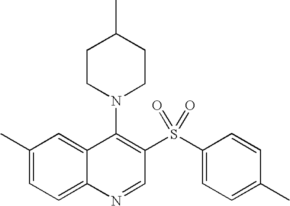 | 395 | *** |
| 47 | 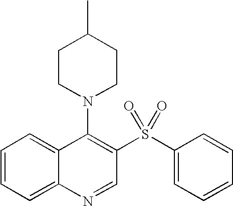 | 367 | *** |
| 48 | 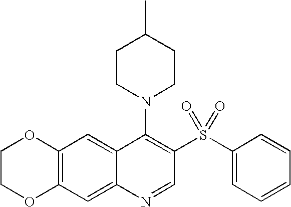 | 425 | *** |
| 49 | 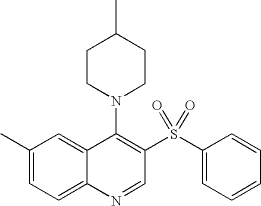 | 381 | * |
| 50 | 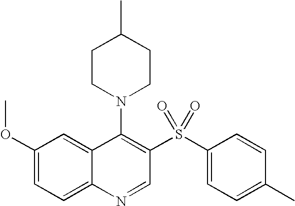 | 411 | * |
| 51 | 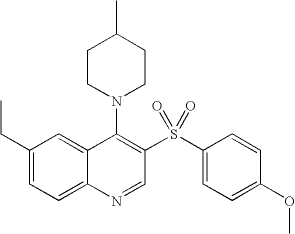 | 425 | *** |
| 52 | 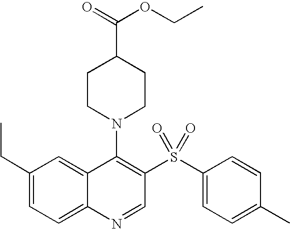 | 466 | * |
| 53 | 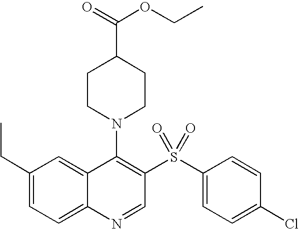 | 487 | *** |
| 54 | 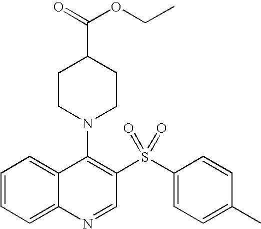 | 439 | * |
| 55 | 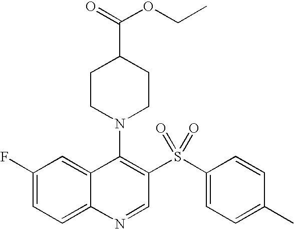 | 457 | * |
| 56 | 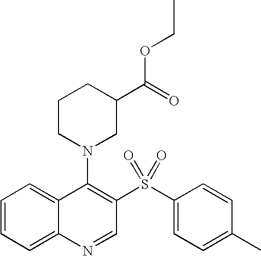 | 439 | * |
| 57 | 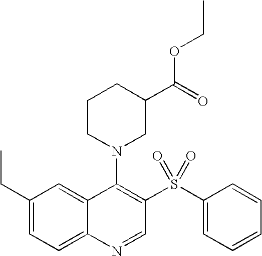 | 453 | * |
| 58 | 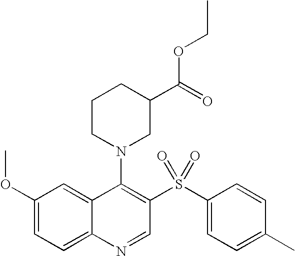 | 469 | * |
| 59 | 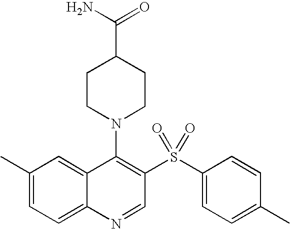 | 424 | * |
| 60 | 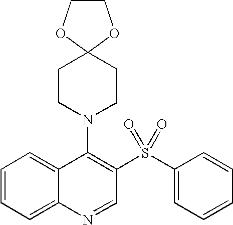 | 411 | * |
| 61 | 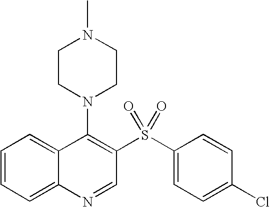 | 402 | * |
| 62 | 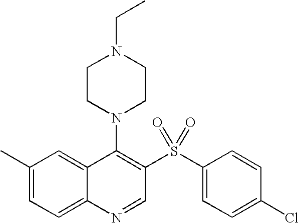 | 430 | * |
| 63 | 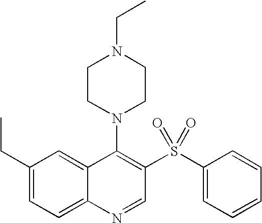 | 410 | * |
| 64 | 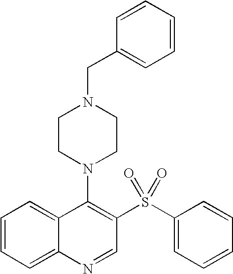 | 444 | * |
| 65 | 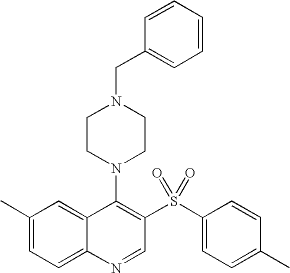 | 472 | * |
| 66 | 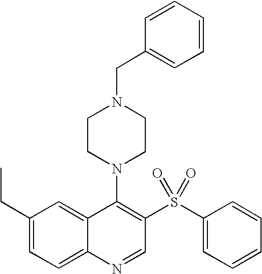 | 472 | * |
| 67 | 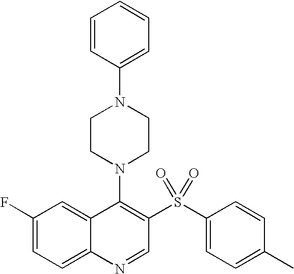 | 462 | * |
| 68 | 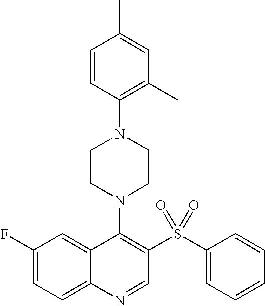 | 476 | * |
| 69 | 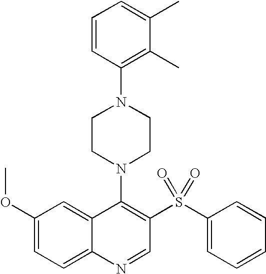 | 488 | * |
| 70 | 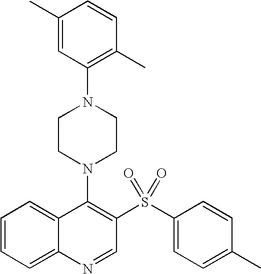 | 472 | * |
| 71 | 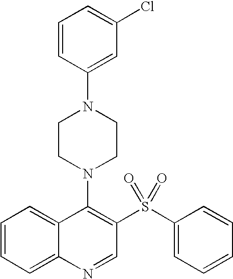 | 464 | * |
| 72 | 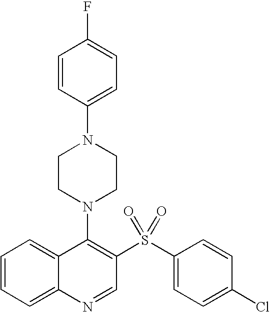 | 482 | * |
| 73 | 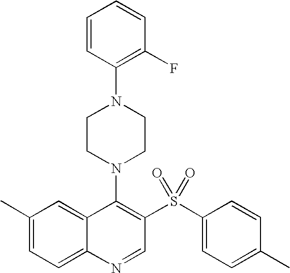 | 476 | * |
| 74 | 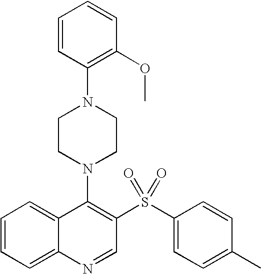 | 474 | * |
| 75 | 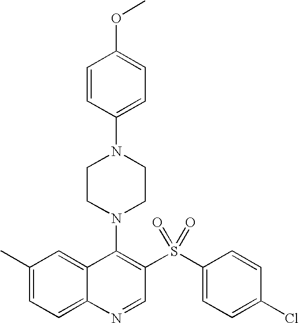 | 508 | * |
| 76 | 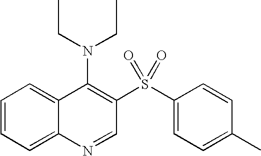 | 355 | ** |
| 77 | 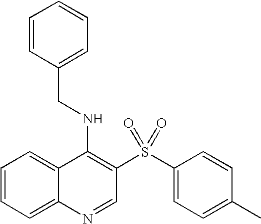 | 389 | *** |
| 78 | 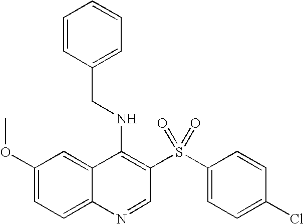 | 439 | * |
| 79 | 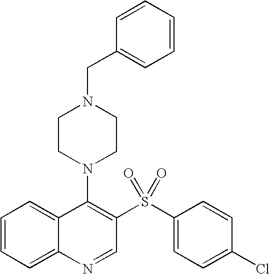 | 478 | ** |
| 80 | 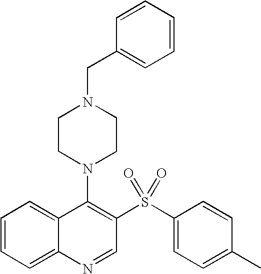 | 458 | * |
| 81 | 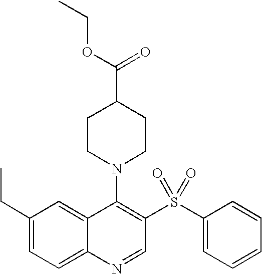 | 453 | * |
| 82 | 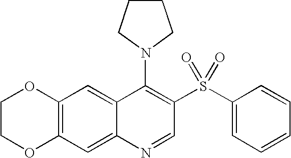 | 397 | * |
| 83 | 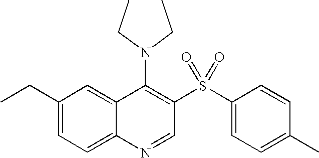 | 383 | * |
| 84 | 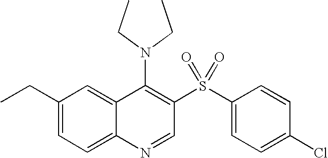 | 403 | * |
| 85 | 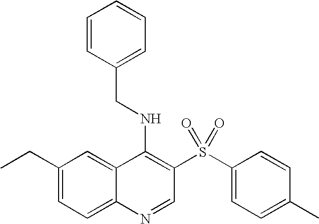 | 417 | * |
| 86 | 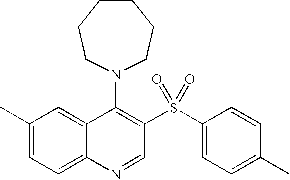 | 395 | * |
| 87 | 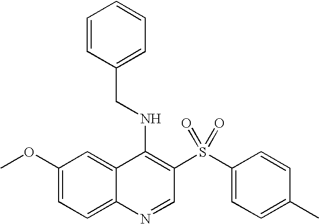 | 419 | * |
| 88 | 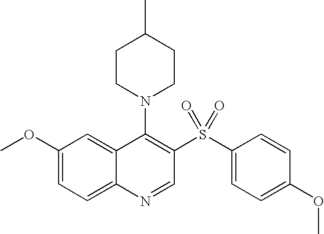 | 427 | * |
| 89 | 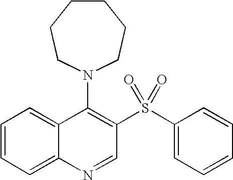 | 367 | ** |
| 90 | 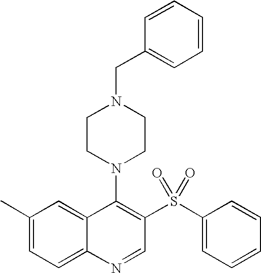 | 458 | * |
| 91 | 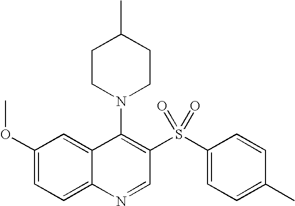 | 411 | * |
| 92 | 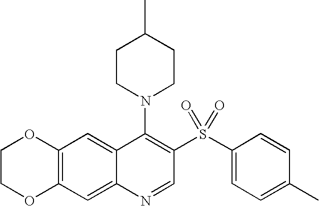 | 439 | * |
| 93 | 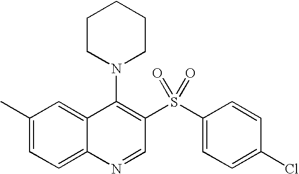 | 401 | * |
| 94 | 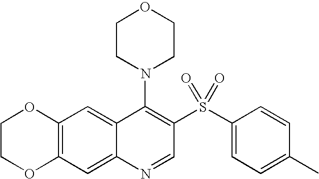 | 427 | * |
| 95 | 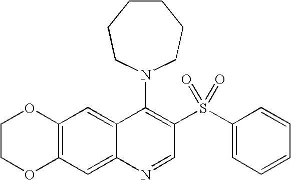 | 425 | * |
| 96 | 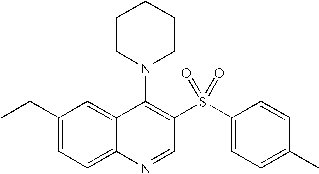 | 395 | * |
| 97 | 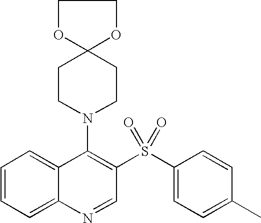 | 425 | * |
| 98 | 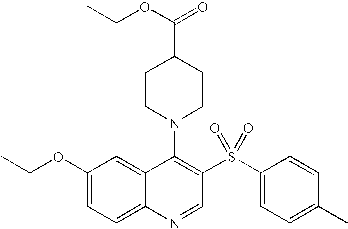 | 483 | * |
| 99 | 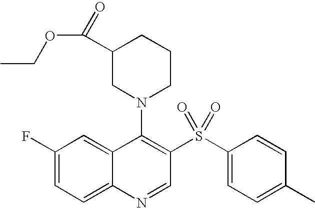 | 457 | * |
| 100 | 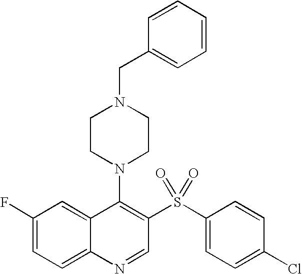 | 496 | * |
| 101 | 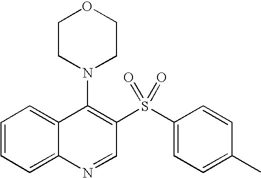 | 369 | * |
| 102 | 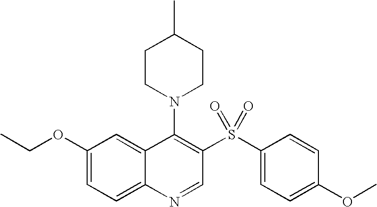 | 441 | * |
| 103 | 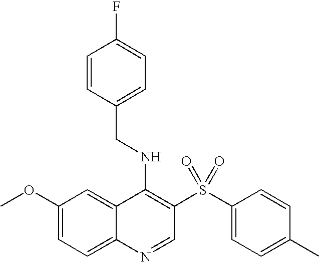 | 437 | * |
| 104 | 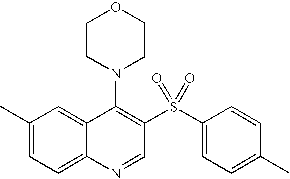 | 383 | * |
| 105 | 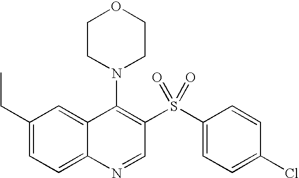 | 417 | * |
| 106 | 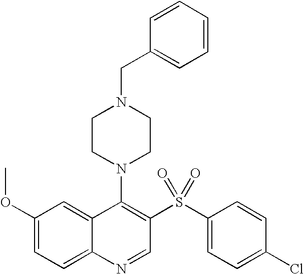 | 508 | * |
| 107 | 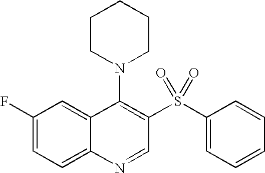 | 371 | * |
| 108 | 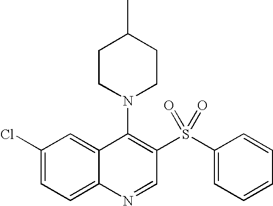 | 401 | * |
| 109 | 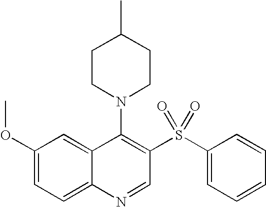 | 397 | * |
| 110 | 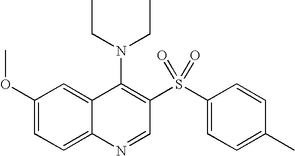 | 385 | * |
| 111 | 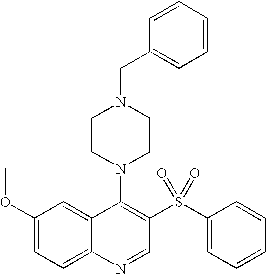 | 474 | * |
| 112 | 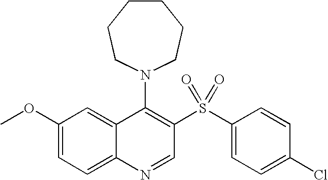 | 431 | * |
| 113 | 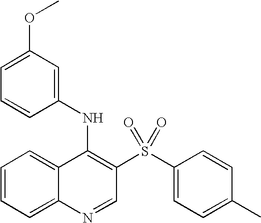 | 405 | * |
| 114 | 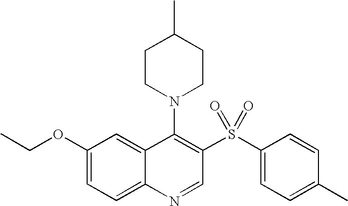 | 425 | * |
| 115 | 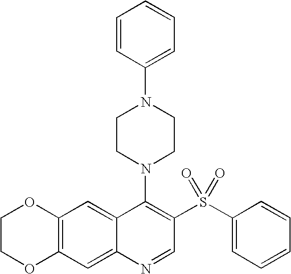 | 488 | * |
| 116 | 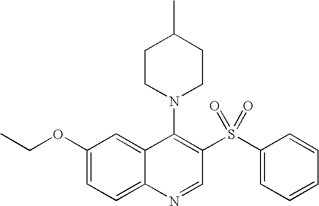 | 411 | * |
| 117 | 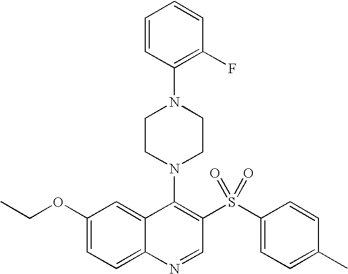 | 506 | * |
| 118 | 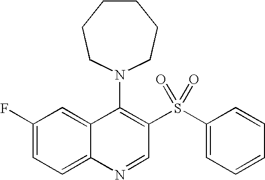 | 385 | * |
| 119 | 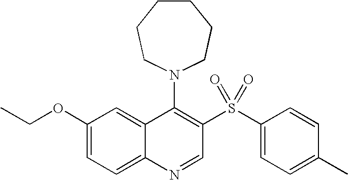 | 425 | * |
| 120 | 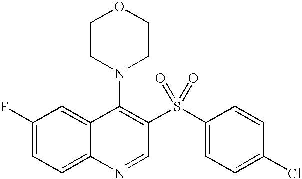 | 407 | * |
| 121 | 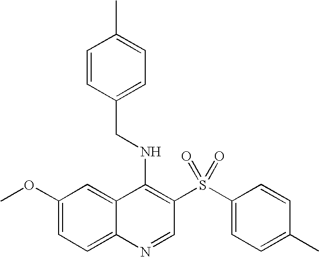 | 433 | * |
| 122 | 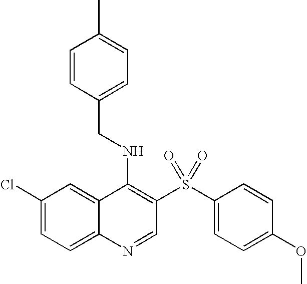 | 453 | * |
| 123 | 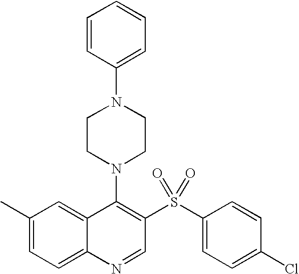 | 478 | * |
| 124 | 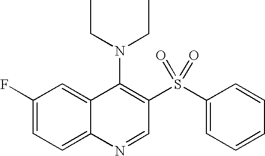 | 359 | * |
| 125 | 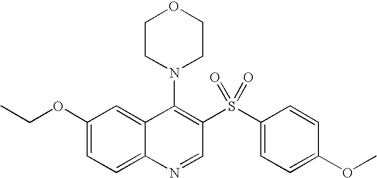 | 429 | * |
| 126 | 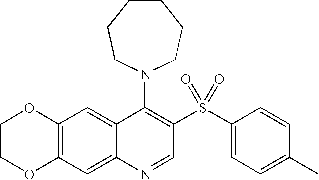 | 441 | * |
| 127 | 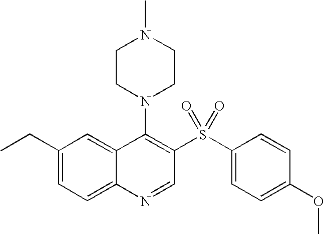 | 426 | * |
| 128 | 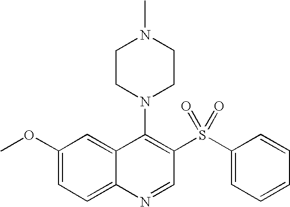 | 398 | * |
| 129 | 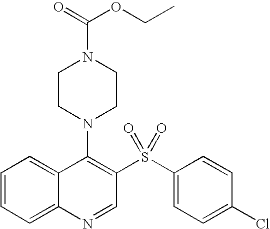 | 460 | * |
| 130 | 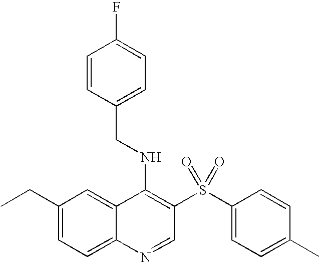 | 435 | * |
| 131 | 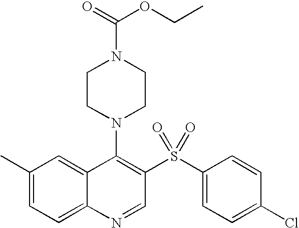 | 474 | ** |
| 132 | 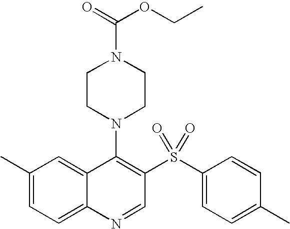 | 454 | * |
| 133 | 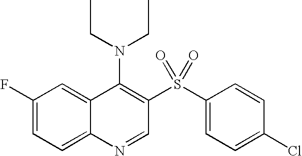 | 393 | * |
| 134 | 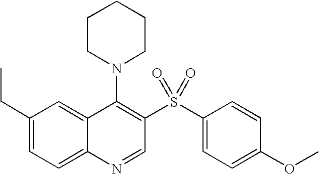 | 411 | * |
| 135 | 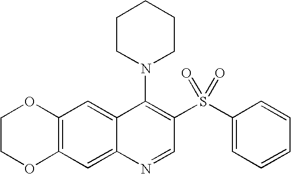 | 411 | * |
| 136 | 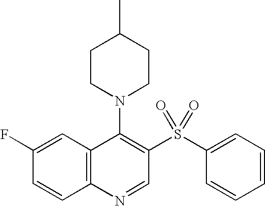 | 385 | * |
| 137 | 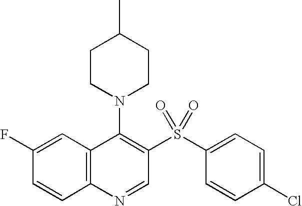 | 419 | *** |
| 138 | 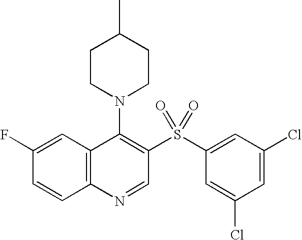 | 454 | ** |
| 139 | 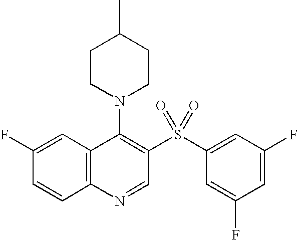 | 421 | * |
| 140 | 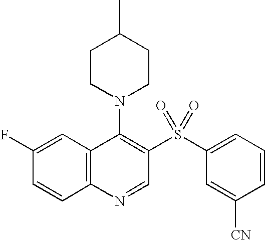 | 410 | *** |
| 141 | 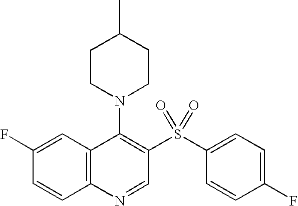 | 403 | *** |
| 142 | 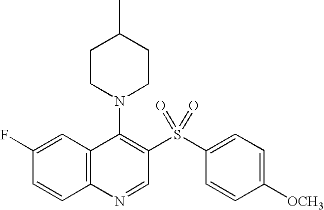 | 415 | *** |
| 143 | 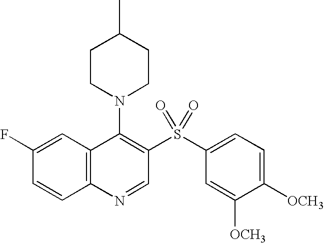 | 445 | ** |
| 144 | 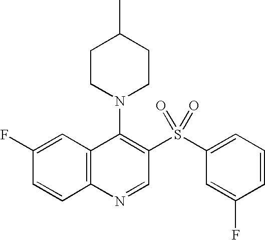 | 403 | ** |
| 145 | 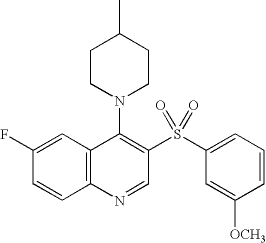 | 415 | *** |
| 146 | 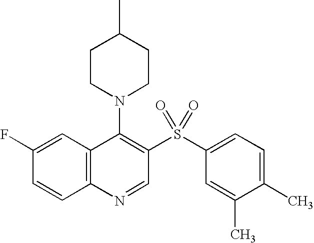 | 413 | *** |
| 147 | 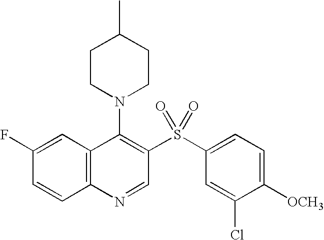 | 449 | *** |
| 148 | 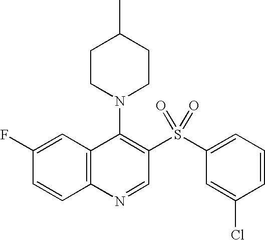 | 419 | *** |
| 149 | 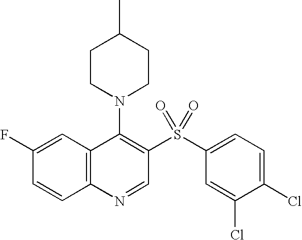 | 454 | *** |
| 150 | 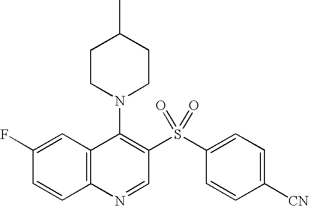 | 410 | ** |
| 151 | 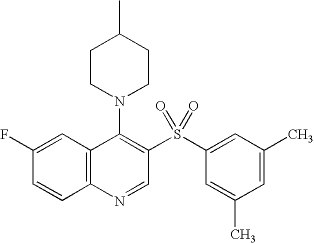 | 413 | *** |
| 152 | 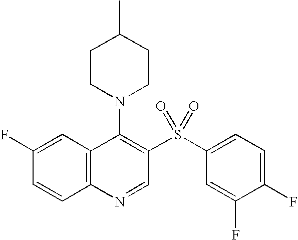 | 421 | *** |
| 153 | 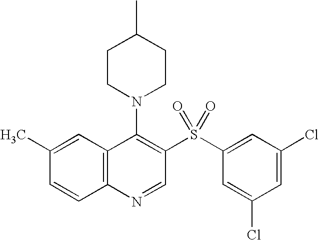 | 450 | ** |
| 154 | 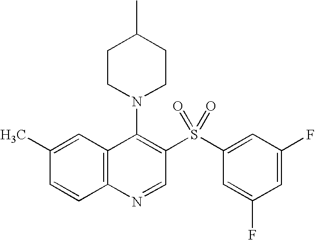 | 417 | ** |
| 155 | 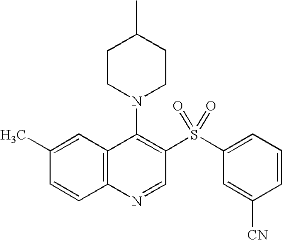 | 406 | * |
| 156 | 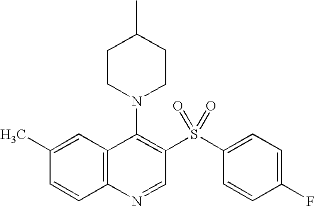 | 399 | *** |
| 157 | 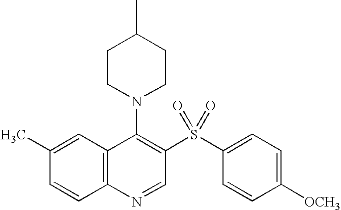 | 411 | ** |
| 158 | 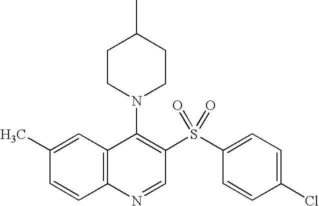 | 415 | ** |
| 159 | 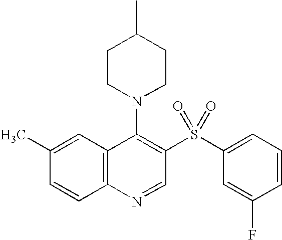 | 399 | *** |
| 160 | 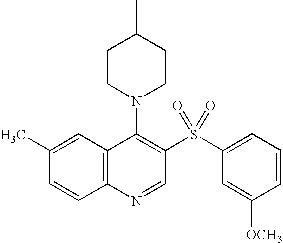 | 411 | *** |
| 161 | 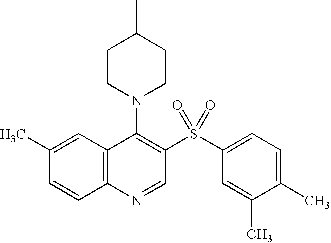 | 409 | *** |
| 162 | 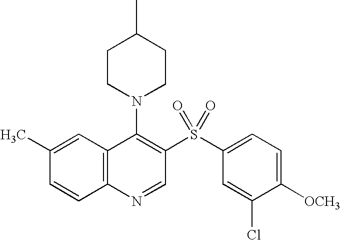 | 445 | *** |
| 163 | 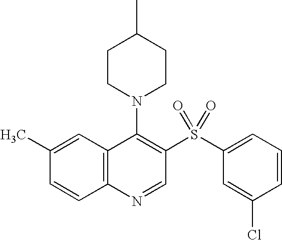 | 415 | *** |
| 164 | 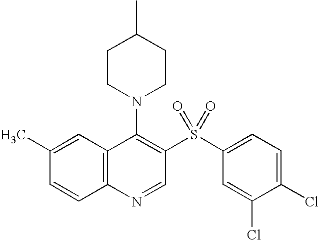 | 450 | *** |
| 165 | 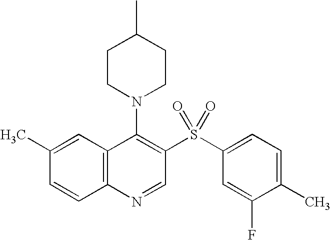 | 413 | *** |
| 166 | 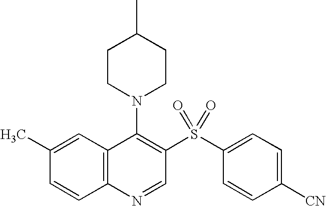 | 406 | ** |
| 167 | 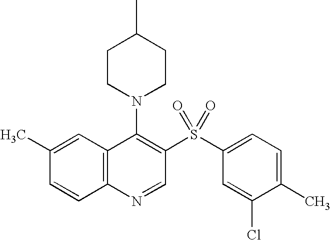 | 429 | *** |
| 168 | 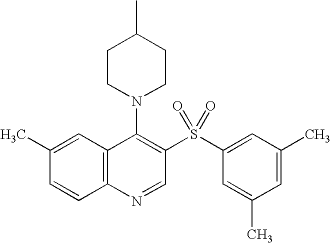 | 409 | ** |
| 169 | 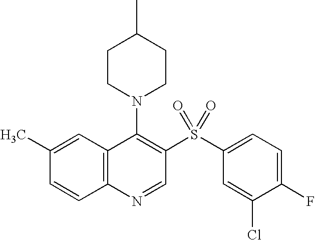 | 434 | *** |
| 170 | 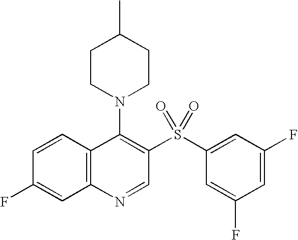 | 421 | ** |
| 171 | 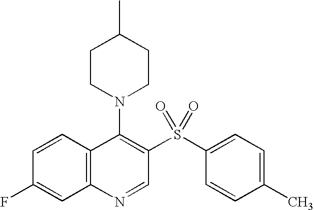 | 399 | *** |
| 172 | 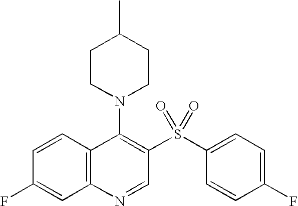 | 403 | ** |
| 173 | 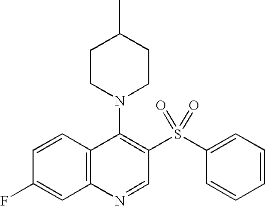 | 385 | *** |
| 174 | 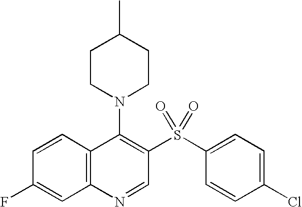 | 419 | *** |
| 175 | 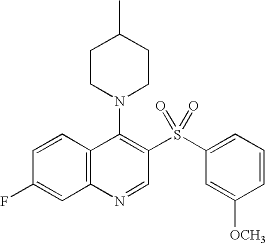 | 415 | *** |
| 176 | 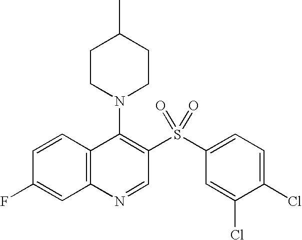 | 454 | *** |
| 177 | 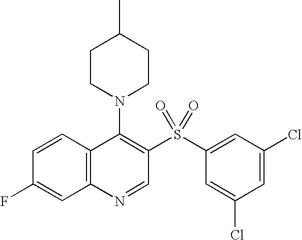 | 454 | *** |
| 178 | 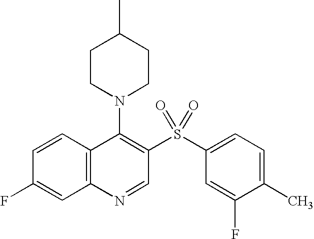 | 417 | *** |
| 179 | 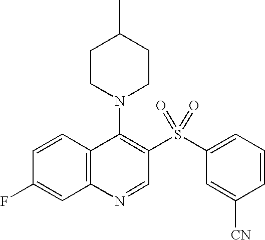 | 410 | *** |
| 180 | 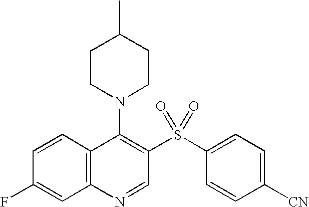 | 410 | *** |
| 181 | 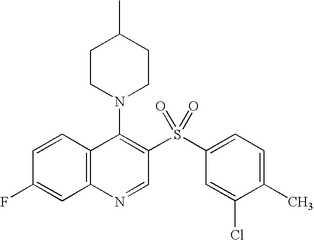 | 434 | *** |
| 182 | 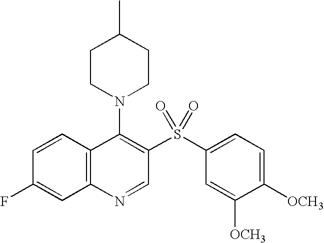 | 445 | *** |
| 183 | 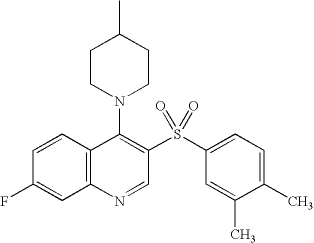 | 413 | *** |
| 184 | 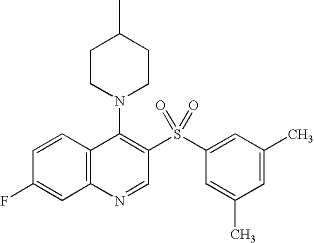 | 413 | ** |
| 185 | 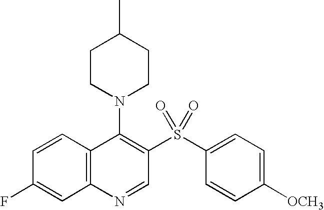 | 415 | *** |
| 186 | 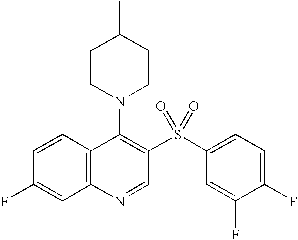 | 421 | *** |
| 187 | 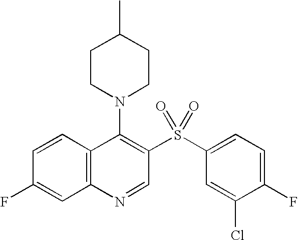 | 437 | *** |
| 188 | 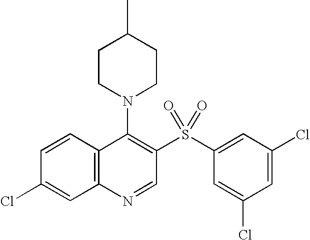 | 471 | *** |
| 189 | 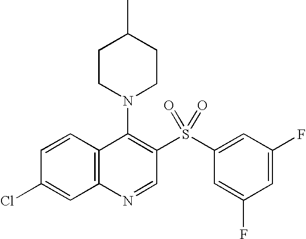 | 437 | *** |
| 190 | 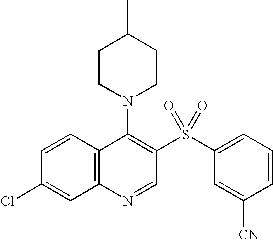 | 426 | *** |
| 191 | 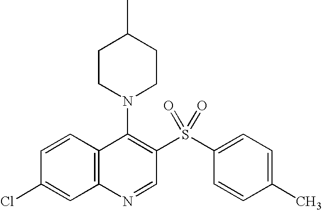 | 415 | *** |
| 192 | 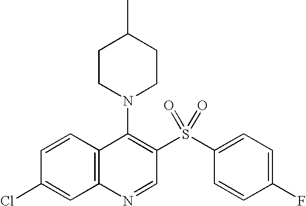 | 419 | *** |
| 193 | 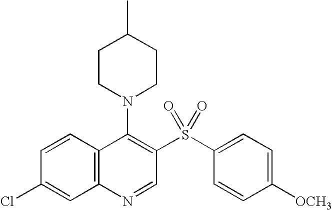 | 431 | ** |
| 194 | 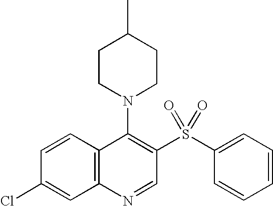 | 401 | *** |
| 195 | 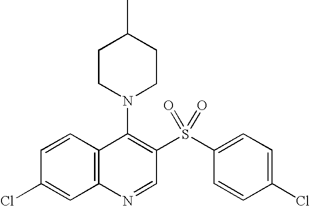 | 436 | *** |
| 196 | 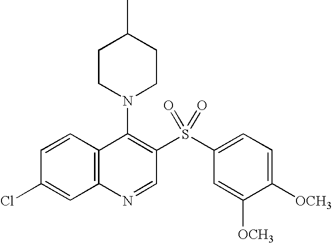 | 461 | *** |
| 197 | 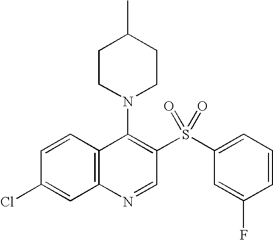 | 419 | *** |
| 198 | 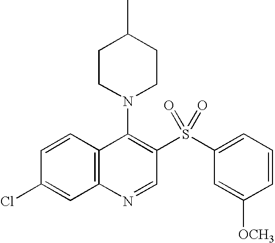 | 431 | *** |
| 199 | 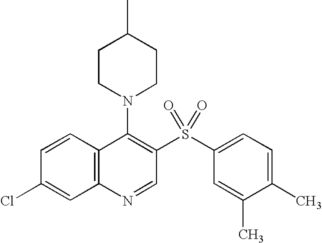 | 429 | *** |
| 200 | 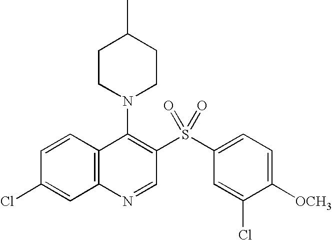 | 466 | *** |
| 201 | 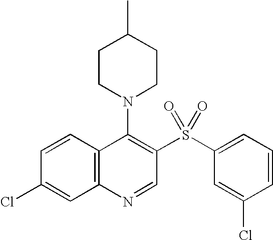 | 436 | *** |
| 202 | 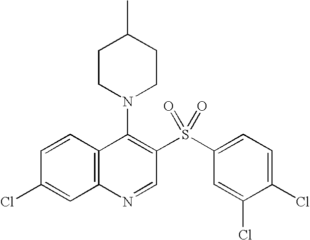 | 471 | *** |
| 203 | 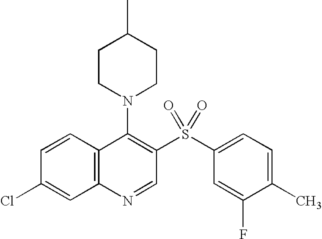 | 434 | *** |
| 204 | 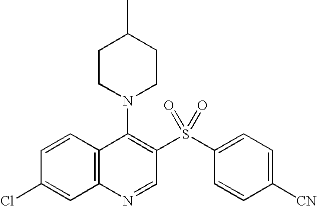 | 426 | ** |
| 205 | 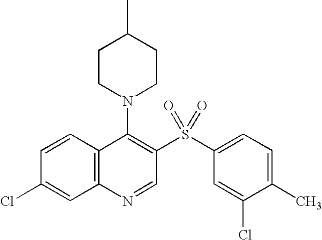 | 450 | *** |
| 206 | 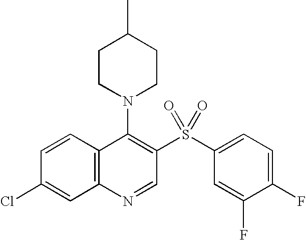 | 437 | *** |
| 207 | 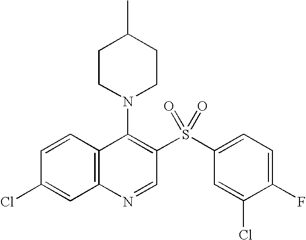 | 454 | *** |
| 208 | 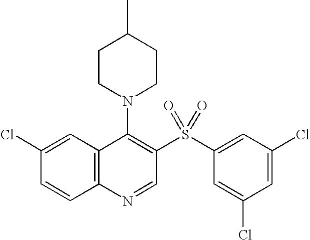 | 471 | *** |
| 209 | 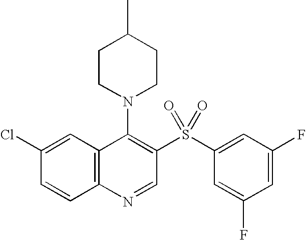 | 437 | *** |
| 210 | 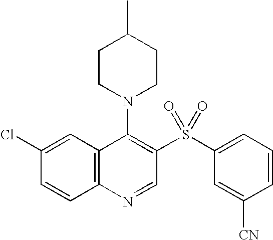 | 426 | *** |
| 211 | 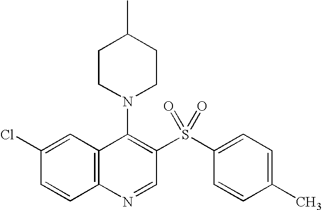 | 415 | *** |
| 212 | 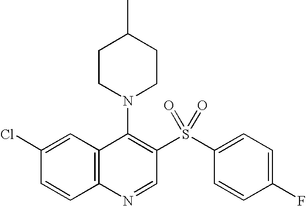 | 419 | *** |
| 213 | 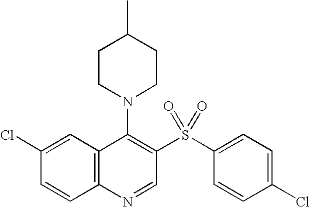 | 436 | *** |
| 214 | 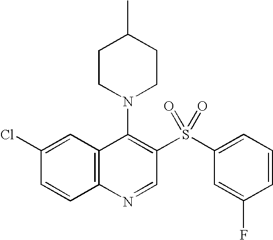 | 419 | ** |
| 215 | 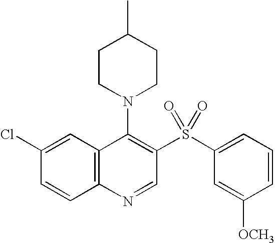 | 431 | *** |
| 216 | 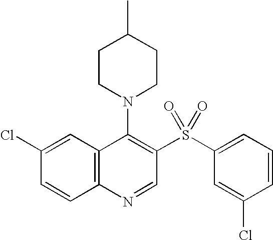 | 436 | * |
| 217 | 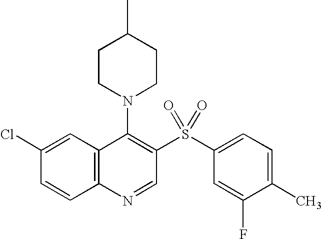 | 434 | ** |
| 218 | 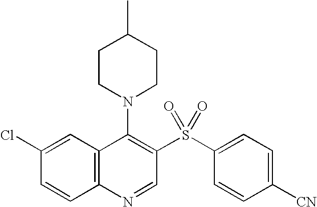 | 426 | * |
| 219 | 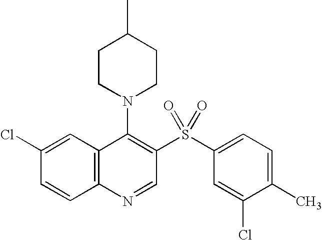 | 450 | ** |
| 220 | 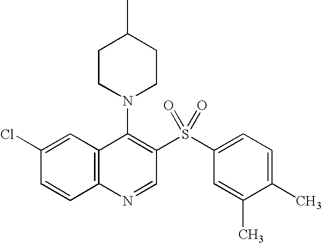 | 429 | *** |
| 221 | 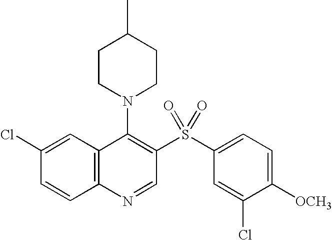 | 466 | * |
| 222 | 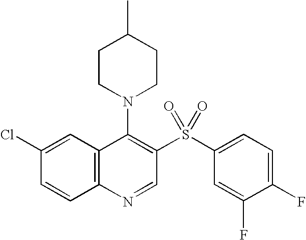 | 437 | ** |
| 223 | 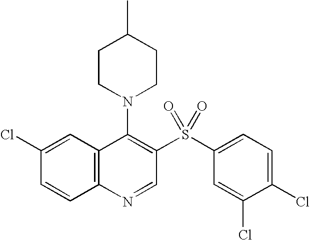 | 471 | *** |
| 224 | 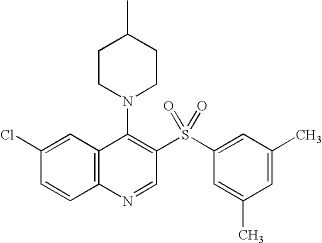 | 429 | * |
| 225 | 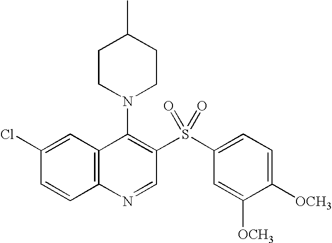 | 461 | ** |
| 226 | 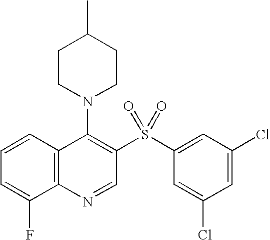 | 454 | *** |
| 227 | 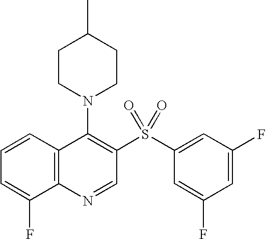 | 421 | * |
| 228 | 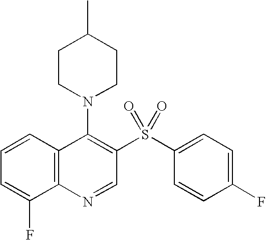 | 403 | * |
| 229 | 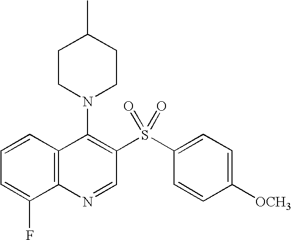 | 415 | ** |
| 230 | 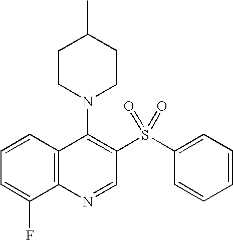 | 385 | * |
| 231 | 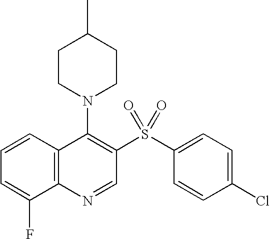 | 419 | *** |
| 232 | 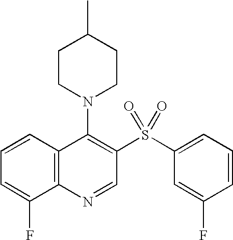 | 403 | ** |
| 233 | 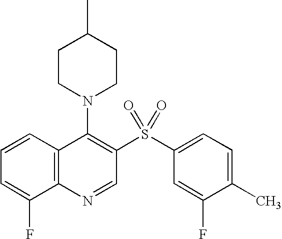 | 417 | *** |
| 234 | 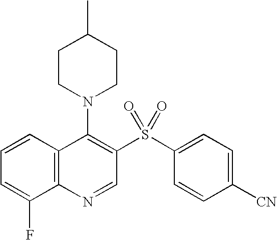 | 410 | *** |
| 235 | 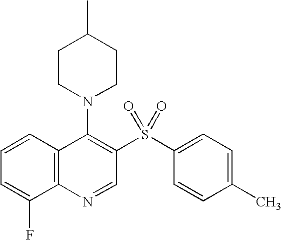 | 399 | *** |
| 236 | 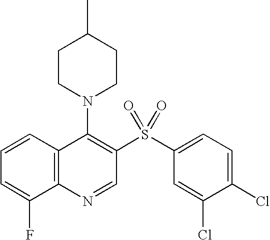 | 454 | *** |
| 237 | 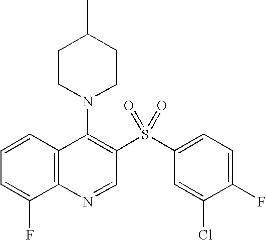 | 437 | *** |
| 238 | 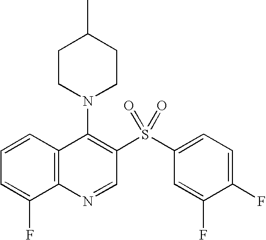 | 421 | *** |
| 239 | 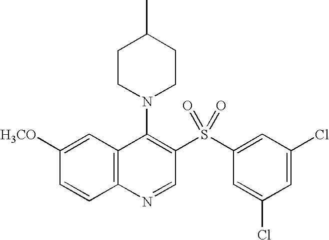 | 466 | *** |
| 240 | 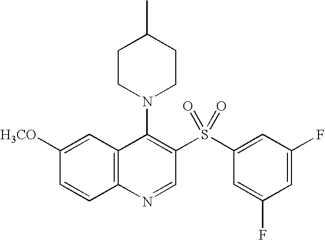 | 433 | * |
| 241 | 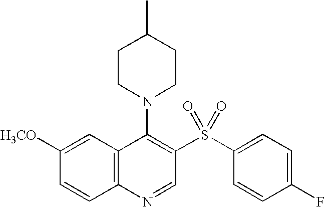 | 415 | * |
| 242 | 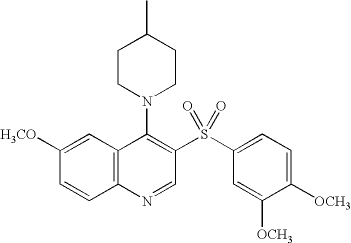 | 457 | * |
| 243 | 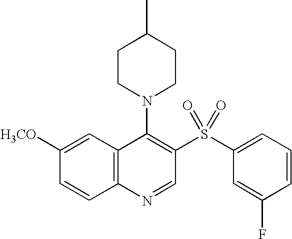 | 415 | ** |
| 244 | 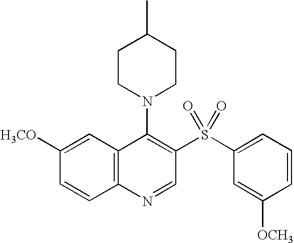 | 427 | * |
| 245 | 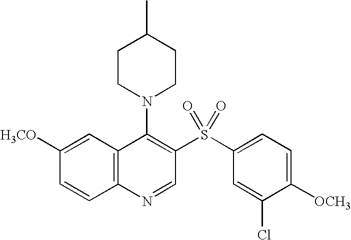 | 461 | * |
| 246 | 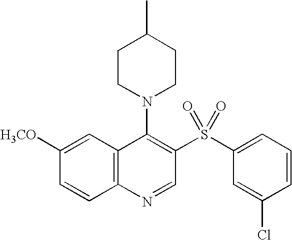 | 432 | * |
| 247 | 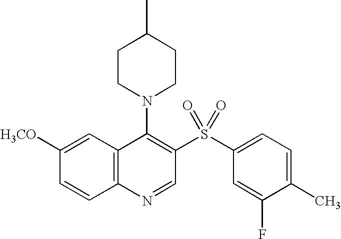 | 429 | ** |
| 248 | 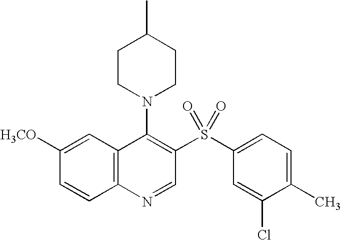 | 445 | *** |
| 249 | 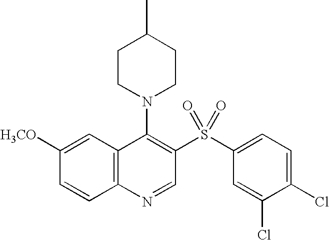 | 466 | *** |
| 250 | 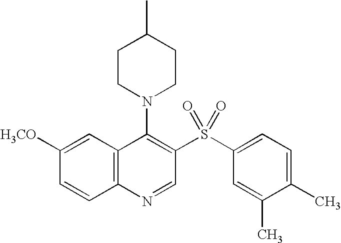 | 425 | ** |
| 251 | 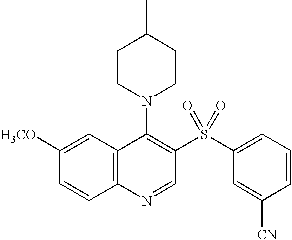 | 432 | *** |
| 252 | 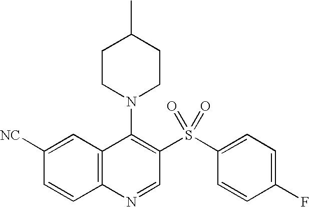 | 410 | ** |
| 253 | 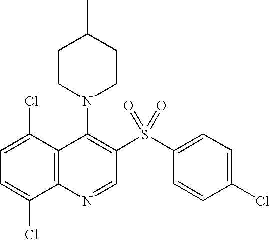 | 471 | ** |
| 254 | 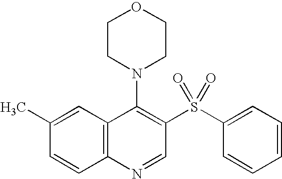 | 369 | *** |
| 255 | 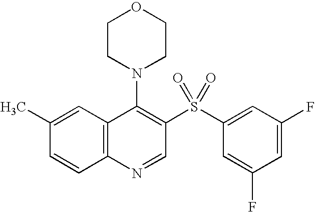 | 405 | * |
| 256 | 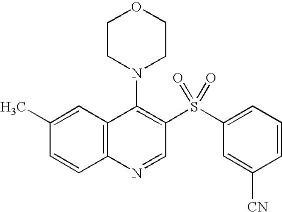 | 394 | ** |
| 257 | 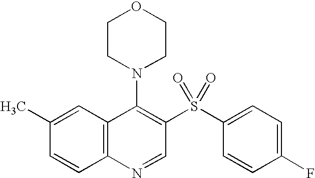 | 387 | * |
| 258 | 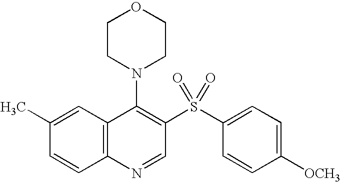 | 399 | * |
| 259 | 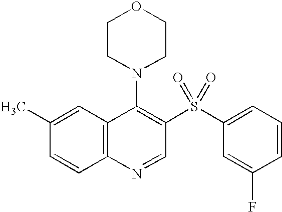 | 387 | * |
| 260 | 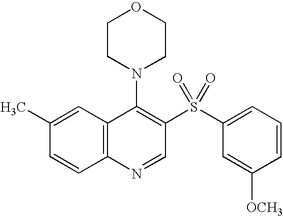 | 399 | ** |
| 261 | 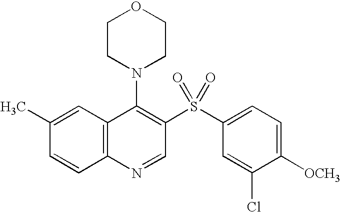 | 433 | ** |
| 262 | 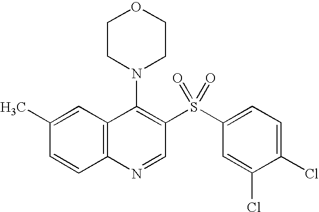 | 438 | ** |
| 263 | 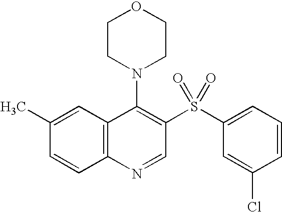 | 403 | * |
| 264 | 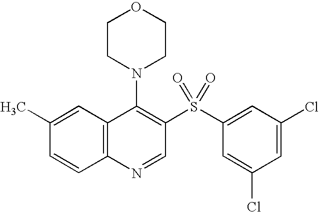 | 438 | ** |
| 265 | 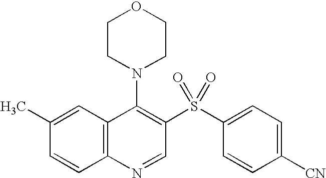 | 394 | * |
| 266 | 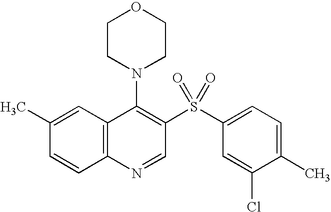 | 417 | ** |
| 267 | 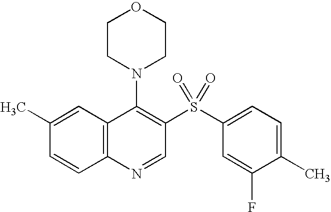 | 401 | * |
| 268 | 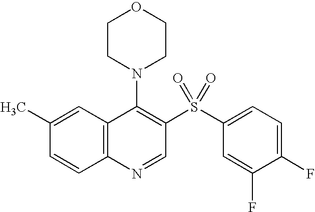 | 405 | * |
| 269 | 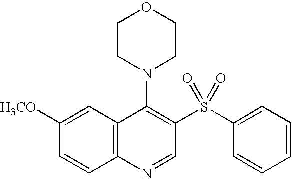 | 385 | ** |
| 270 | 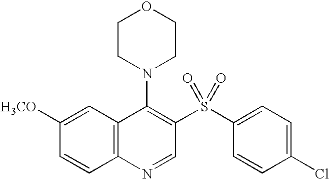 | 419 | *** |
| 271 | 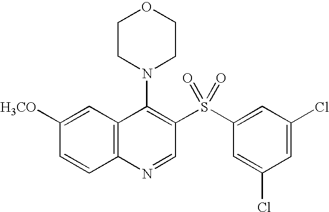 | 454 | * |
| 272 | 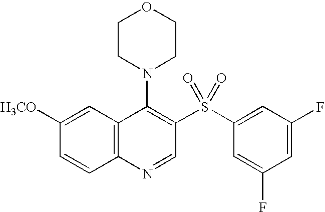 | 421 | * |
| 273 | 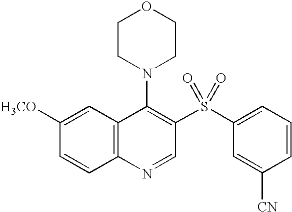 | 410 | * |
| 274 | 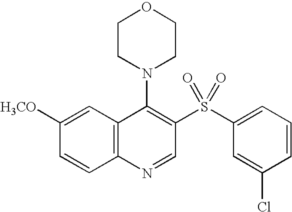 | 419 | * |
| 275 | 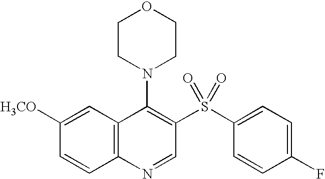 | 403 | * |
| 276 | 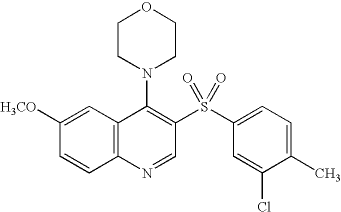 | 433 | * |
| 277 | 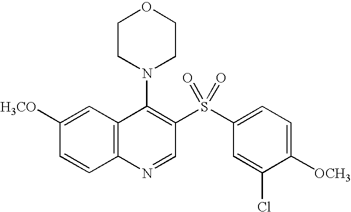 | 449 | * |
| 278 | 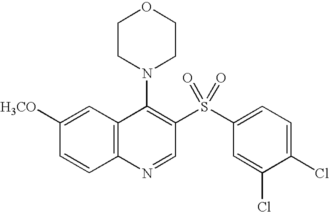 | 454 | * |
| 279 | 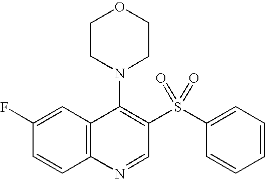 | 373 | *** |
| 280 | 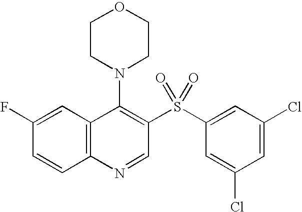 | 442 | * |
| 281 | 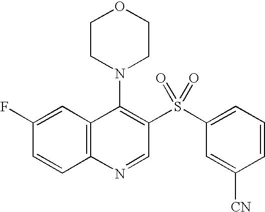 | 398 | * |
| 282 | 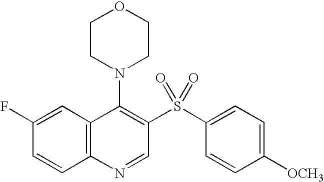 | 403 | * |
| 283 | 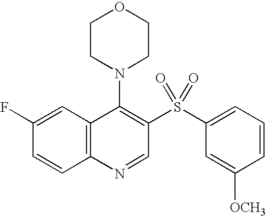 | 403 | * |
| 284 | 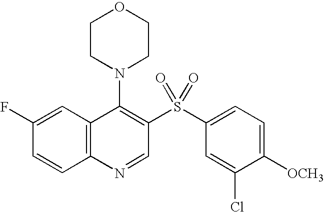 | 437 | * |
| 285 | 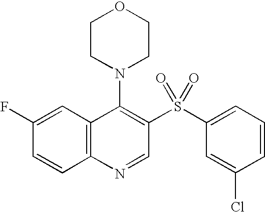 | 407 | * |
| 286 | 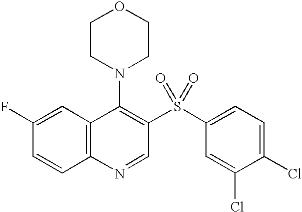 | 442 | ** |
| 287 | 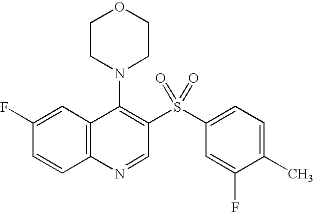 | 405 | * |
| 288 | 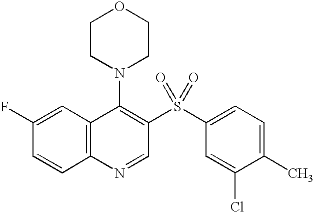 | 422 | ** |
| 289 | 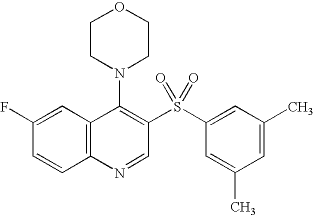 | 401 | * |
| 290 | 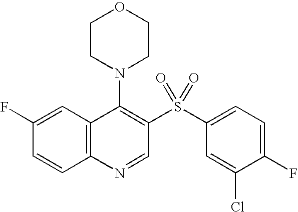 | 425 | ** |
| 291 | 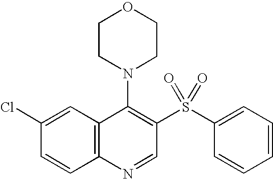 | 389 | * |
| 292 | 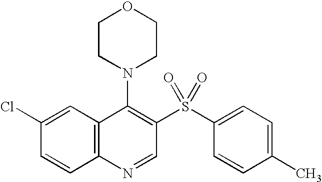 | 403 | *** |
| 293 | 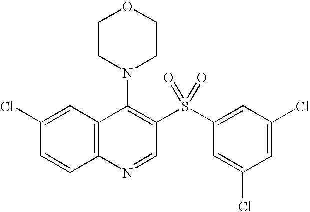 | 459 | * |
| 294 | 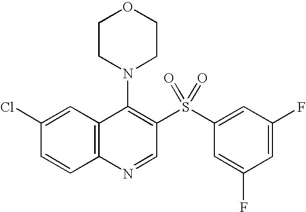 | 425 | * |
| 295 | 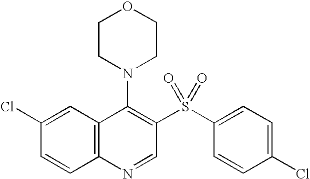 | 424 | * |
| 296 | 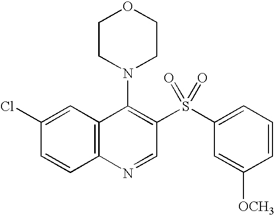 | 419 | * |
| 297 | 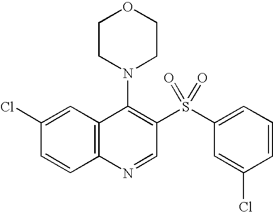 | 424 | * |
| 298 | 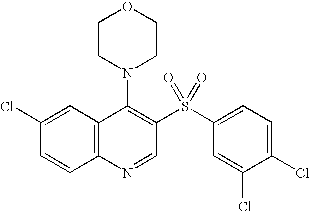 | 459 | ** |
| 299 | 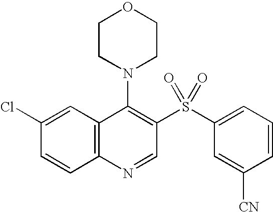 | 414 | * |
| 300 | 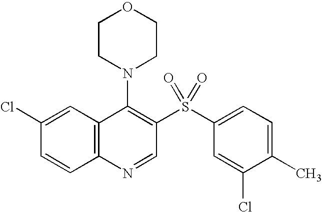 | 438 | ** |
| 301 | 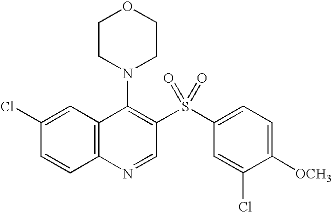 | 454 | * |
| 302 | 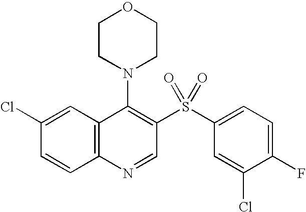 | 442 | * |
| 303 | 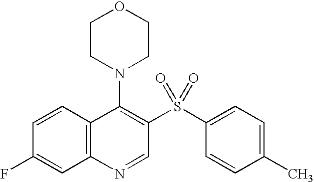 | 387 | * |
| 304 | 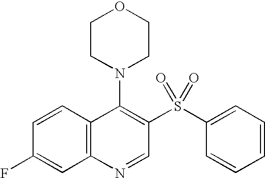 | 373 | * |
| 305 | 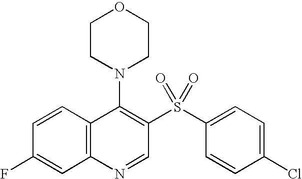 | 407 | * |
| 306 | 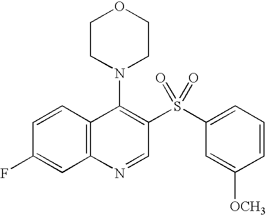 | 403 | ** |
| 307 | 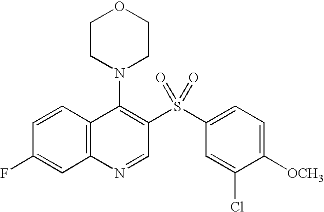 | 437 | ** |
| 308 | 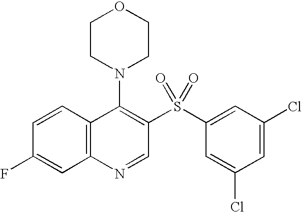 | 442 | ** |
| 309 | 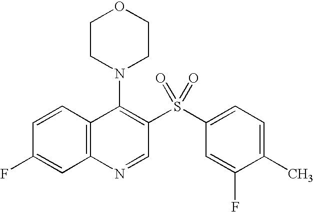 | 405 | ** |
| 310 | 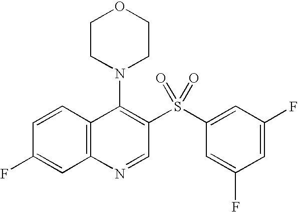 | 409 | * |
| 311 | 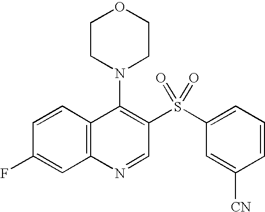 | 398 | ** |
| 312 | 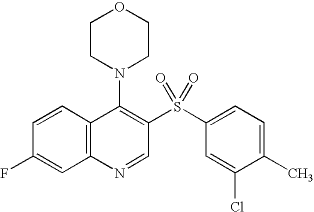 | 421 | *** |
| 313 | 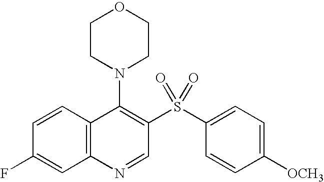 | 403 | * |
| 314 | 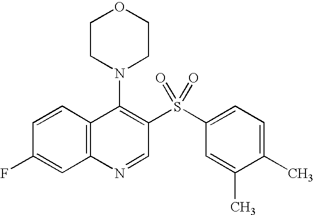 | 401 | ** |
| 315 | 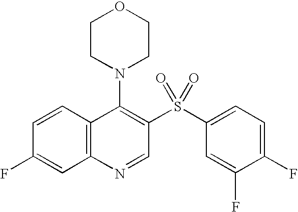 | 409 | ** |
| 316 | 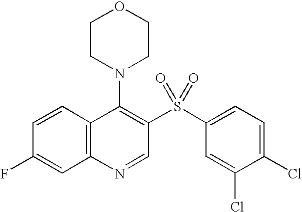 | 442 | *** |
| 317 | 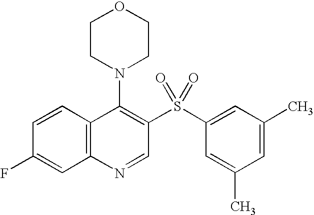 | 401 | ** |
| 318 | 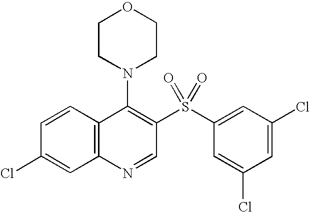 | 459 | *** |
| 319 | 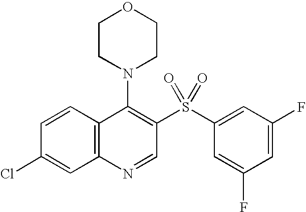 | 425 | ** |
| 320 | 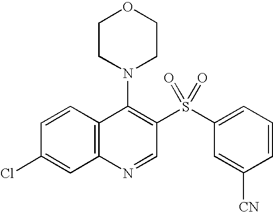 | 414 | ** |
| 321 | 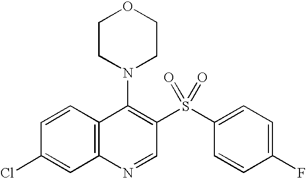 | 407 | * |
| 322 | 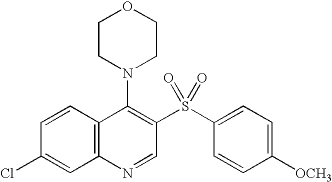 | 419 | * |
| 323 | 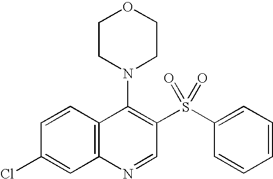 | 389 | * |
| 324 | 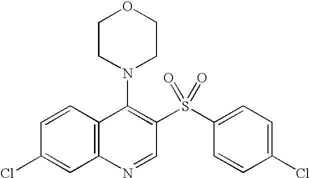 | 424 | ** |
| 325 | 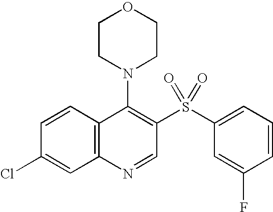 | 407 | * |
| 326 | 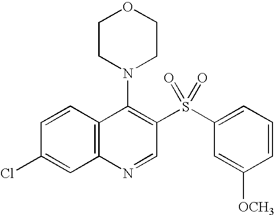 | 419 | ** |
| 327 | 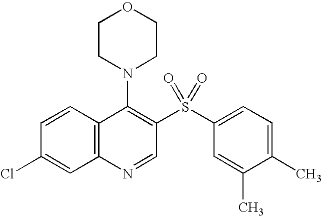 | 417 | *** |
| 328 | 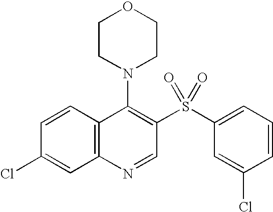 | 424 | *** |
| 329 | 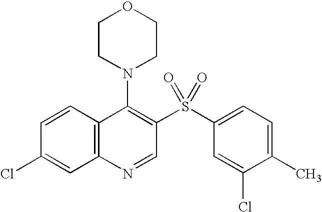 | 438 | *** |
| 330 | 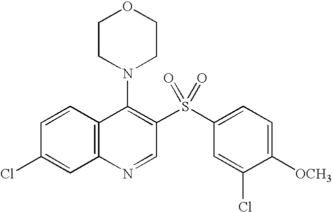 | 454 | ** |
| 331 | 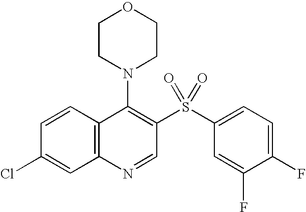 | 425 | * |
| 332 | 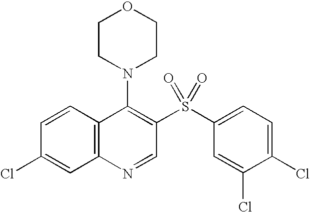 | 459 | *** |
| 333 | 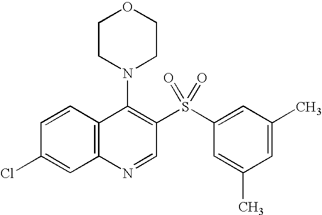 | 417 | * |
| 334 | 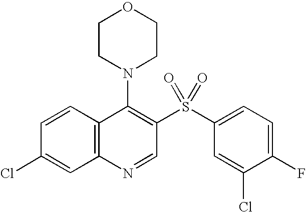 | 441 | *** |
| 335 | 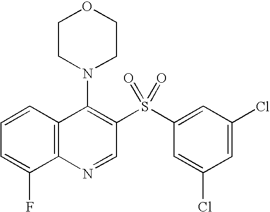 | 441 | ** |
| 336 | 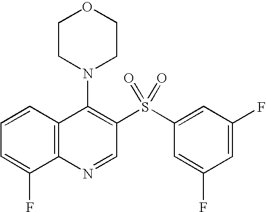 | 409 | ** |
| 337 | 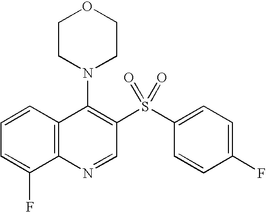 | 391 | * |
| 338 | 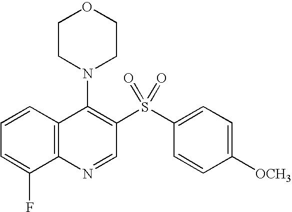 | 403 | * |
| 339 | 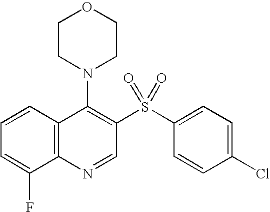 | 407 | ** |
| 340 | 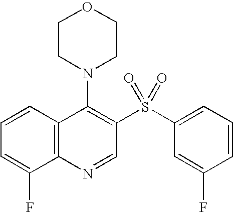 | 391 | * |
| 341 | 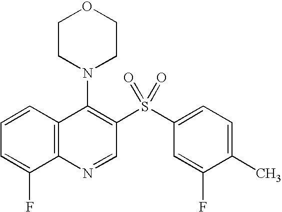 | 405 | ** |
| 342 | 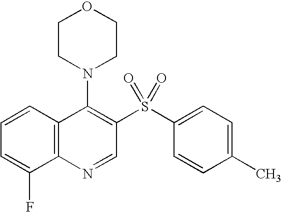 | 387 | * |
| 343 | 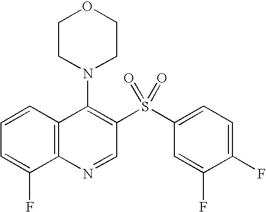 | 409 | ** |
| 344 | 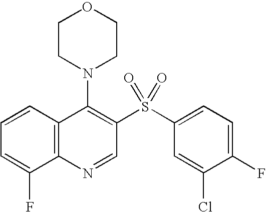 | 425 | *** |
| 345 | 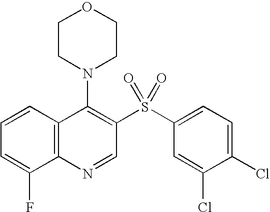 | 442 | *** |
| 346 | 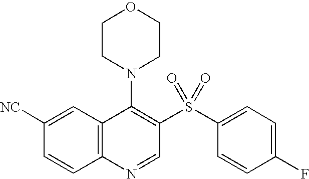 | 398 | ** |
| 347 | 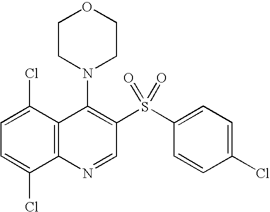 | 459 | ** |
| 348 | 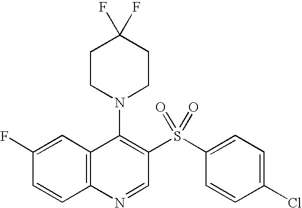 | 441 | ** |
| 349 | 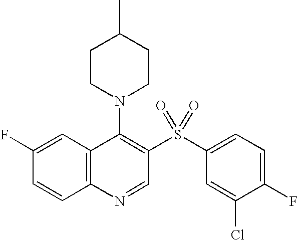 | 437 | ** |
| 350 | 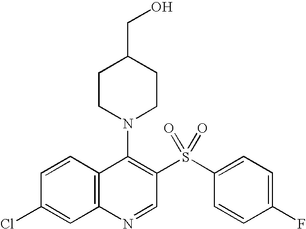 | 435 | ** |
| 351 | 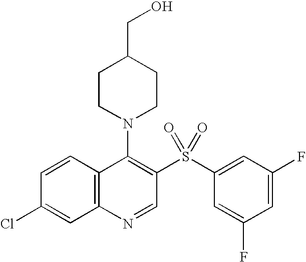 | 453 | ** |

*** Kᵢ < 200 nM
** 200 nM < Kᵢ < 500 nM
* 500 nM < Kᵢ

### Example 6

### Preparation of Pharmaceutical Compositions

### a) Tablets:

0.01-50% of active ingredient of formula (I), 15-50% of lactose, 15-50% of potato starch, 5-15% of polyvinyl pyrrolidone, 1-5% of talc, 0.01-3% of magnesium stearate, 1-3% of colloid silicon dioxide and 2-7% of ultraamylopectin were mixed, then granulated by wet granulation and pressed to tablets.

b) Dragées, Film Coated Tablets:

The tablets made according to the method described above were coated by a layer consisting of entero- or gastrosolvent film, or of sugar and talc. The dragées were polished by a mixture of beeswax and carnuba wax.

### c) Capsules:

0.01-50% of active ingredient of formula (I), 1-5% of sodium lauryl sulfate, 15-50% of starch, 15-50% of lactose, 1-3% of colloid silicon dioxide and 0.01-3% of magnesium stearate were thoroughly mixed, the mixture was passed through a sieve and filled in hard gelatin capsules.

### d) Suspensions:

Ingredients: 0.01-15% of active ingredient of formula (I), 0.1-2% of sodium hydroxide, 0.1-3% of citric acid, 0.05-0.2% of nipagin (sodium methyl 4-hydroxybenzoate), 0.005-0.02% of nipasol, 0.01-0.5% of carbopol (polyacrilic acid), 0.1-5% of 96% ethanol, 0.1-1% of flavoring agent, 20-70% of sorbitol (70% aqueous solution) and 30-50% of distilled water.

To solution of nipagin and citric acid in 20 ml of distilled water, carbopol was added in small portions under vigorous stirring, and the solution was left to stand for 10-12 h. Then the sodium hydroxide in 1 ml of distilled water, the aqueous solution of sorbitol and finally the ethanolic raspberry flavor were added with stirring. To this carrier the active ingredient was added in small portions and suspended with an immersing homogenizator. Finally the suspension was filled up to the desired final volume with distilled water and the suspension syrup was passed through a colloid milling equipment.

### e) Suppositories:

For each suppository 0.01-15% of active ingredient of formula (I) and 1-20% of lactose were thoroughly mixed, then 50-95% of adeps pro suppository (for example Witepsol 4) was melted, cooled to 35° C. and the mixture of active ingredient and lactose was mixed in it with homogenizator. The obtained mixture was mould in cooled forms.

### f) Lyophilized Powder Ampoule Compositions:

A 5% solution of mannitol or lactose was made with bidistilled water for injection use, and the solution was filtered so as to have sterile solution. A 0.01-5% solution of the active ingredient of formula (I) was also made with bidistilled water for injection use, and this solution was filtered so as to have sterile solution. These two solutions were mixed under aseptic conditions, filled in 1 ml portions into ampoules, the content of the ampoules was lyophilized, and the ampoules were sealed under nitrogen. The contents of the ampoules were dissolved in sterile water or 0.9% (physiological) sterile aqueous sodium chloride solution before administration.

1-26. (canceled)

27. A compound having the formula (I): 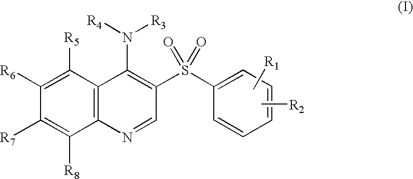
wherein:
R₁ and R₂ are independently selected from hydrogen, halogen, alkyl, alkoxy, cyano, an optionally substituted amino group, and a saturated heterocyclyl group containing N as a heteroatom;
R₃ and R₄ are independently selected from hydrogen, alkyl, and an aryl group optionally substituted with at least one substituent selected from halogen, alkyl, and alkoxy, or
R₃ and R₄ together with the N atom to which they are attached form a C₅₋₇ heterocyclyl group containing 1 or 2 heteroatom(s) selected from N and O, and which heterocyclyl is optionally substituted by a halogen, alkyl, alkylhydroxy, alkyloxycarbonyl, aminocarbonyl, —OCH₂CH₂O—, benzyl or substituted phenyl; and,
R₅, R₆, R₇ and R₈ are independently selected from hydrogen, halogen, alkyl, alkoxy, and a cyano group, or
R₆ and R₇ together with the atoms to which they are attached form an unsaturated heterocyclyl group; or enantiomers, racemates, diastereomers, or pharmaceutically acceptable salts thereof.

28. A compound having the formula (I): 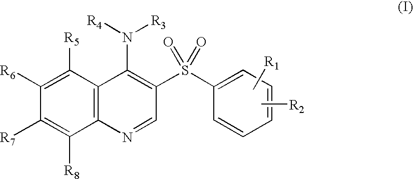
wherein:
R₁ and R₂ are independently selected from hydrogen, halogen, C₁₋₄ alkyl, C₁₋₄ alkoxy, cyano, an optionally substituted amino group, and a saturated heterocyclyl group containing N as a heteroatom;
R₃ and R₄ are independently selected from hydrogen, C₁₋₄ alkyl, and an aryl group optionally substituted with at least one substituent selected from halogen, C₁₋₄ alkyl, and C₁₋₄ alkoxy, or
R₃ and R₄ together with the N atom to which they are attached form a C₅₋₇ heterocyclyl containing 1 or 2 heteroatom(s) selected from N and O, and which is optionally substituted with a substituent selected from halogen, C₁₋₄ alkyl, C₁₋₄ alkylhydroxy, alkyloxycarbonyl, aminocarbonyl, —OCH₂CH₂O—, benzyl and phenyl, which phenyl itself is optionally substituted by 1 or 2 substituents selected from halogen, C₁₋₄ alkyl, and C₁₋₄ alkoxy; and,
R₅, R₆, R₇ and R₈ are independently selected from hydrogen, halogen, C₁₋₄ alkyl, C₁₋₄ alkoxy, and a cyano group, or
R₆ and R₇ together with the atoms to which they are attached form an unsaturated 5 to 7 membered heterocyclyl group containing 1 or 2 O atom(s);
or enantiomers, racemates, diastereomers, or pharmaceutically acceptable salts thereof.

29. A compound having the formula (I): 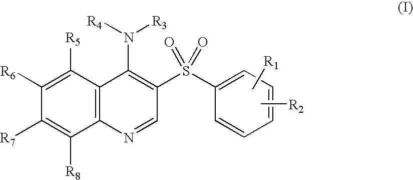
wherein:
R₁ and R₂ are independently selected from hydrogen, chloro, fluoro, C₁₋₂ alkyl, C₁₋₂ alkoxy, cyano and a piperidinyl group;
R₃ and R₄ are independently selected from hydrogen, C₁₋₂ alkyl, a benzyl group optionally substituted by 1 or 2 groups independently selected from halogen, C₁₋₄ alkyl, and C₁₋₄ alkoxy, or
R₃ and R₄ together with the N atom to which they are attached form a pyrrolidinyl, homopiperidinyl, morpholinyl group or piperidinyl group which may be optionally substituted by a substituent selected from halogen, C₁₋₄ alkyl, hydroxymethyl, alkyloxycarbonyl, aminocarbonyl, —OCH₂CH₂O—, and a piperazinyl group which may be substituted at N(4) by a substituent selected from C₁₋₄ alkyl, benzyl, alkyloxycarbonyl and phenyl, which phenyl may be optionally substituted by 1 or 2 groups selected from halogen, C₁₋₄ alkyl, and alkoxy; and,
R₅, R₆, R₇ and R₈ are independently selected from hydrogen, chloro, fluoro, C₁₋₂ alkyl, C₁₋₂ alkoxy, and a cyano group, or
R₆ and R₇ together with the atoms to which they are attached form a 2,3-dihydro-[1,4]dioxine or 2,5-dihydro-furan ring;
or enantiomers, racemates, diastereomers, or pharmaceutically acceptable salts thereof.

30. A compound according to claim 1 selected from:
3-(3,4-Dimethyl-benzenesulfonyl)-4-(morpholin-4-yl)quinoline;
3-(4-Methyl-benzenesulfonyl)-4-(3-methyl-piperidin-1-yl)-quinoline;
3-(3,4-Dimethyl-benzenesulfonyl)-4-(4-methyl-piperidin-1-yl)-quinoline;
3-(4-Methyl-benzenesulfonyl)-4-(4-methyl-piperidin)-quinoline;
3-Benzenesulfonyl-4-(piperidin-1-yl)-quinoline;
3-(4-Methyl-benzenesulfonyl)-4-(piperidin-1-yl)-quinoline;
4-Benzylamino-3-(4-methyl-benzenesulfonyl)-quinoline;
6-Ethyl-4-(4-methyl-piperidin-1-yl)-3-(4-methoxy-benzenesulfonyl)-quinoline;
6-Fluoro-3-(4-methyl-benzenesulfonyl)-4-(4-methyl-piperidin-1-yl)-quinoline;
6-Ethoxy-3-(4-chloro-benzenesulfonyl)-4-(4-fluoro-benzylamino)-quinoline;
4-(Azepan-1-yl)-3-(4-methyl-benzenesulfonyl)-quinoline;
4-(Azepan-1-yl)-3-(4-chloro-benzenesulfonyl)-quinoline;
6-Methyl-3-(4-methyl-benzenesulfonyl)-4-(4-methyl-piperidin-1-yl)-quinoline;
4-(4-Methylpiperidin-1-yl)-3-benzenesulfonyl-quinoline;
9-(4-methyl-piperidin-1-yl)-8-benzenesulfonyl-2,3-dihydro-[1,4]dioxino[2,3-g]quinoline;
6-Ethyl-4-(4-ethyloxycarbonyl-piperidin-1-yl)-3-(4-chloro-benzenesulfonyl)-quinoline;
4-Diethylamino-3-(4-methyl-benzenesulfonyl)-quinoline;
4-(4-Benzyl-piperazin-1-yl)-3-(4-chloro-benzenesulfonyl)-quinoline;
4-(Azepan-1-yl)-3-benzenesulfonyl-quinoline;
3-(3-Cyano-benzenesulfonyl)-6-fluoro-4-(4-methyl-piperidin-1-yl)-quinoline;
6-Fluoro-3-(4-methoxy-benzenesulfonyl)-4-(4-methyl-piperidin-1-yl)-quinoline;
6-Fluoro-3-(3-methoxy-benzenesulfonyl)-4-(4-methyl-piperidin-1-yl)-quinoline;
6-Fluoro-3-(3,4-dmethyl-benzenesulfonyl)-4-(4-methyl-piperidin-1-yl)-quinoline;
3-(3-Chloro-4-methoxy-benzenesulfonyl)-6-fluoro-4-(4-methyl-piperidin-1-yl)-quinoline;
3-(3-Chloro-4-fluoro-benzenesulfonyl)-6-fluoro-4-(4-methyl-piperidin-1-yl)-quinoline;
3-(3,4-Dichloro-benzenesulfonyl)-6-fluoro-4-(4-methyl-piperidin-1-yl)-quinoline;
3-(3-Chloro-benzenesulfonyl)-6-fluoro-4-(4-methyl-piperidin-1-yl)-quinoline;
3-(4-Chloro-benzenesulfonyl)-6-methyl-4-(4-methyl-piperidin-1-yl)-quinoline;
3-(3-Fluoro-benzenesulfonyl)-6-methyl-4-(4-methyl-piperidin-1-yl)-quinoline;
3-(3-Methoxy-benzenesulfonyl)-6-methyl-4-(4-methyl-piperidin-1-yl)-quinoline;
3-(3,4-Dimethyl-benzenesulfonyl)-6-methyl-4-(4-methyl-piperidin-1-yl)-quinoline;
3-(3-Chloro-4-methoxy-benzenesulfonyl)-6-methyl-4-(4-methyl-piperidin-1-yl)-quinoline;
3-(3-Chloro-benzenesulfonyl)-6-methyl-4-(4-methyl-piperidin-1-yl)-quinoline;
3-(3-Fluoro-4-methyl-benzenesulfonyl)-6-methyl-4-(4-methyl-piperidin-1-yl)-quinoline;
3-(3-Chloro-4-methyl-benzenesulfonyl)-6-methyl-4-(4-methyl-piperidin-1-yl)-quinoline;
3-(3-Chloro-4-fluoro-benzenesulfonyl)-6-methyl-4-(4-methyl-piperidin-1-yl)-quinoline;
3-(3,4-Dichloro-benzenesulfonyl)-7-fluoro-4-(4-methyl-piperidin-1-yl)-quinoline;
7-Fluoro-3-(3-cyano-benzenesulfonyl)-4-(4-methyl-piperidin-1-yl)-quinoline;
7-Fluoro-3-(4-cyano-benzenesulfonyl)-4-(4-methyl-piperidin-1-yl)-quinoline;
3-(3-Chloro-4-methyl-benzenesulfonyl)-7-fluoro-4-(4-methyl-piperidin-1-yl)-quinoline;
7-Fluoro-3-(3-methoxy-benzenesulfonyl)-4-(4-methyl-piperidin-1-yl)-quinoline;
3-(3,4-Difluoro-benzenesulfonyl)-7-fluoro-4-(4-methyl-piperidin-1-yl)-quinoline;
3-(3-chloro-4-fluoro-benzenesulfonyl)-7-fluoro-4-(4-methyl-piperidin-1-yl)-quinoline;
7-Chloro-3-(3,5-dichloro-benzenesulfonyl)-4-(4-methyl-piperidin-1-yl)-quinoline;
7-Chloro-3-(3,5-difluoro-benzenesulfonyl)-4-(4-methyl-piperidin-1-yl)-quinoline;
7-Chloro-3-(3-cyano-benzenesulfonyl)-4-(4-methyl-piperidin-1-yl)-quinoline;
7-Chloro-3-(4-methyl-benzenesulfonyl)-4-(4-methyl-piperidin-1-yl)-quinoline;
7-Chloro-3-(4-fluoro-benzenesulfonyl)-4-(4-methyl-piperidin-1-yl)-quinoline;
3-Benzenesulfonyl-7-Chloro-4-(4-methyl-piperidin-1-yl)-quinoline;
7-Chloro-3-(4-chloro-benzenesulfonyl)-4-(4-methyl-piperidin-1-yl)-quinoline;
7-Chloro-3-(3,4-dimethoxy-benzenesulfonyl)-4-(4-methyl-piperidin-1-yl)-quinoline;
7-Chloro-3-(3-fluoro-benzenesulfonyl)-4-(4-methyl-piperidin-1-yl)-quinoline;
7-Chloro-3-(3-methoxy-benzenesulfonyl)-4-(4-methyl-piperidin-1-yl)-quinoline;
7-Chloro-3-(3-chloro-4-methoxy-benzenesulfonyl)-4-(4-methyl-piperidin-1-yl)-quinoline;
7-Chloro-3-(3-chloro-benzenesulfonyl)-4-(4-methyl-piperidin-1-yl)-quinoline;
7-Chloro-3-(3-chloro-4-fluoro-benzenesulfonyl)-4-(4-methyl-piperidin-1-yl)-quinoline;
6-Chloro-3-(3,5-difluoro-benzenesulfonyl)-4-(4-methyl-piperidin-1-yl)-quinoline;
6-Chloro-3-(4-methyl-benzenesulfonyl)-4-(4-methyl-piperidin-1-yl)-quinoline;
6-Chloro-3-(4-chloro-benzenesulfonyl)-4-(4-methyl-piperidin-1-yl)-quinoline;
8-Fluoro-3-(3-fluoro-4-methyl-benzenesulfonyl)-4-(4-methyl-piperidin-1-yl)-quinoline;
8-Fluoro-3-(4-methyl-benzenesulfonyl)-4-(4-methyl-piperidin-1-yl)-quinoline;
3-(3,4-Dichloro-benzenesulfonyl)-8-fluoro-4-(4-methyl-piperidin-1-yl)-quinoline;
3-(3-chloro-4-fluoro-benzenesulfonyl)-8-fluoro-4-(4-methyl-piperidin-1-yl)-quinoline;
3-(3,4-Difluoro-benzenesulfonyl)-8-fluoro-4-(4-methyl-piperidin-1-yl)-quinoline;
3-Benzenesulfonyl-6-methyl-4-(morpholin-1-yl)-quinoline;
3-(3-Chloro-benzenesulfonyl)-6-methoxy-4-(morpholin-1-yl)-quinoline;
3-Benzenesulfonyl-6-fluoro-4-(morpholin-1-yl)-quinoline;
6-Chloro-3-(4-chloro-benzenesulfonyl)-4-(morpholin-1-yl)-quinoline;
3-(3-Chloro-4-methyl-benzenesulfonyl)-7-fluoro-4-(morpholin-1-yl)-quinoline;
3-(3,4-Dichloro-benzenesulfonyl)-7-fluoro-4-(morpholin-1-yl)-quinoline;
7-Chloro-3-(3,5-dichloro-benzenesulfonyl)-4-(morpholin-1-yl)-quinoline;
7-Chloro-3-(3,4-dimethyl-benzenesulfonyl)-4-(morpholin-1-yl)-quinoline;
7-Chloro-3-(3-chloro-benzenesulfonyl)-4-(morpholin-1-yl)-quinoline;
7-Chloro-3-(3-chloro-4-methyl-benzenesulfonyl)-4-(morpholin-1-yl)-quinoline;
7-Chloro-3-(3,4-dichloro-benzenesulfonyl)-4-(morpholin-1-yl)-quinoline; and,
7-Chloro-3-(3-chloro-4-fluoro-benzenesulfonyl)-4-(morpholin-1-yl)-quinoline.

31. A process for the preparation of a compound of formula (I): 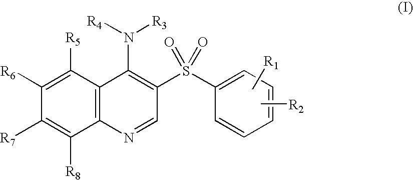
wherein:
R₁ and R₂ are independently selected from hydrogen, halogen, alkyl, alkoxy, cyano, an optionally substituted amino group, and a saturated heterocyclyl group containing N as a heteroatom;
R₃ and R₄ are independently selected from hydrogen, alkyl, and an aryl group optionally substituted with at least one substituent selected from halogen, alkyl, and alkoxy, or
R₃ and R₄ together with the N atom to which they are attached form a C₅₋₇ heterocyclyl group containing 1 or 2 heteroatom(s) selected from N and O, and which may be optionally substituted by a substituent selected from halogen, alkyl, alkylhydroxy, alkyloxycarbonyl, aminocarbonyl, —OCH₂CH₂O—, benzyl and a substituted phenyl group;
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts of the compounds thereof, comprising:
a. converting a compound of formula (VI): 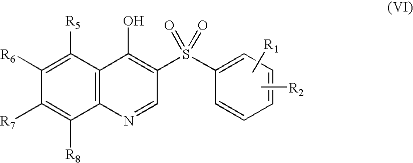
wherein R₁, R₂, R₅, R₆, R₇ and R₈ are as defined above for the compound of formula (I), to a compound of formula (VII): 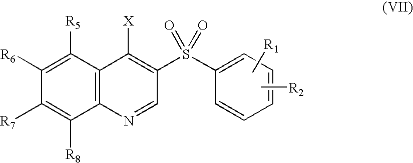
wherein X is selected from halogen, benzenesulfonyloxy and trifluoromethanesulfonyloxy, and
R₁, R₂, R₅, R₆, R₇ and R₈ are as defined above for a compound of formula (I); thereafter reacting the compound of formula (VII) with a compound of formula (VIII): 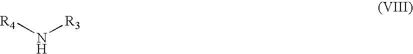
wherein R₃ and R₄ are as defined above for a compound of formula (I), to give a compound of formula (I); and, optionally, forming enantiomers, racemates, diastereomers, or pharmaceutically acceptable salts of the compounds of formula (I); or
b.) reacting a compound of formula (XV) 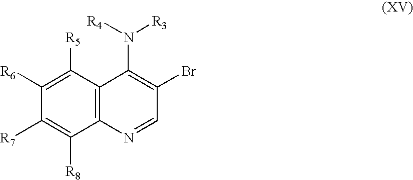
wherein R₃, R₄, R₅, R₆, R₇ and R₈ are as defined above for a compound of formula (I), with a compound of formula (III): 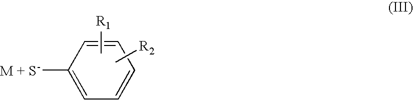
wherein M is selected from alkali and alkaline-earth metals, R₁ and R₂ are as defined above for a compound of formula (I), to give a compound of formula (XVI): 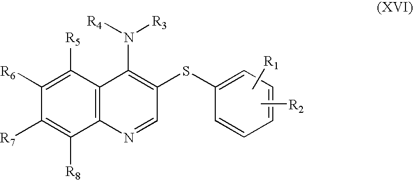
wherein R₁, R₂, R₃, R₄, R₅, R₆, R₇ and R₈ are as defined above for a compound of formula (I);
thereafter oxidizing the compound of formula (XVI) to obtain a compound of formula (XVII): 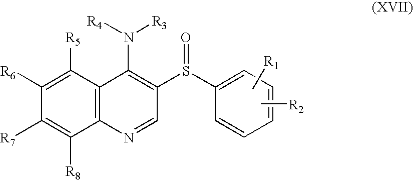
wherein R₁, R₂, R₃, R₄, R₅, R₆, R₇ and R₈ are as defined above for a compound of formula (I); thereafter oxidizing a compound of formula (XVII) to obtain a compound of formula (I): and, optionally, forming enantiomers, racemates, diastereomers, or pharmaceutically acceptable salts of the compounds of formula (I); or
c.) interconverting one compound of formula (I), wherein the meaning of R₁, R₂, R₃, R₄, R₅, R₆, R₇ and R₈ is as defined above for the formula (I), to a different compound of formula (I), wherein the meaning of R₁, R₂, R₃, R₄, R₅, R₆, R₇ and R₈ is as described above for the formula (I);
where appropriate, separating the enantiomers, racemates, or diastereomers of compounds of formula (I), wherein the meaning of R₁, R₂, R₃, R₄, R₅, R₆, R₇ and R₈ is as described above for the formula (I);
and optionally, thereafter forming salts, hydrates, or solvates of compounds of formula (I).

32. A process for the preparation of a compound of formula (VI): 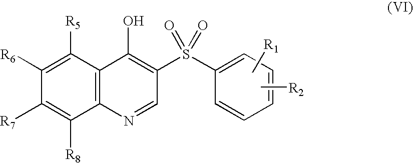
wherein:
R₁ and R₂ are independently selected from hydrogen, halogen, alkyl, alkoxy, cyano, an optionally substituted amino group, and a saturated heterocyclyl group containing N as a heteroatom;
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof, comprising:
a. reacting a compound of formula (II): 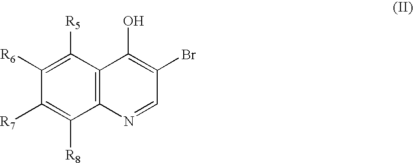
wherein R₅, R₆, R₇ and R₈ are as defined above for a compound of formula (I), with a compound of formula (III): 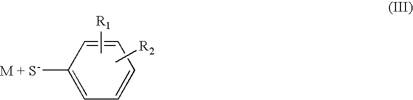
wherein M is selected from alkali metals and alkaline-earth metals, and R₁ and R₂ are as defined above for a compound of formula (I), to give a compound of formula (IV): 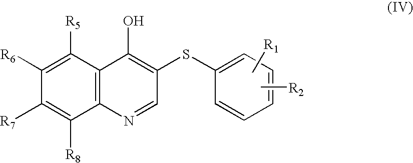
wherein R₁, R₂, R₅, R₆, R₇ and R₈ are as defined above for a compound of formula (I); thereafter oxidizing the compound of formula (IV) to obtain a compound of formula (V): 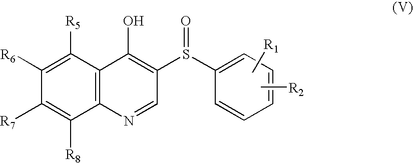
wherein R₁, R₂, R₅, R₆, R₇ and R₈ are as defined above for a compound of formula (I); thereafter oxidizing the compound of formula (V) to obtain a compound of formula (VI), and optionally forming enantiomers, racemates, diastereomers, or pharmaceutically acceptable salts of the compounds of formula (VI); or,
b. reacting a compound of formula (IX): 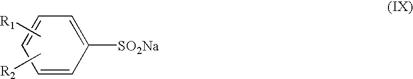
whererein R₁ and R₂ are as defined above for a compound of formula (I), with α-halogen-acetic acid esters of formula (X)
Hlg-CH₂—COOR₉ (X)
wherein Hlg is halogen and R₉ is an ethyl or methyl group, to obtain a compound of formula (XI) 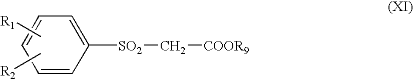
wherein R₁ and R₂ are as defined above for a compound of formula (I) and R₉ is as defined above for compounds of formula (X); reacting the compound of formula (XI) with a trialkyl orthoformate of formula (XII):
CH(OR₁₀)₃ (XII)
wherein R₁₀ is an ethyl or methyl group, to obtain a compound of formula (XIII): 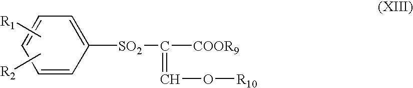
wherein R₁ and R₂ are as defined above for a compound of formula (I), R₉ is as defined above for compounds of formula (X) and R₁₀ is as defined above for compounds of formula (XII); reacting the compound of formula (XIII) with an aniline derivative of formula (XIV): 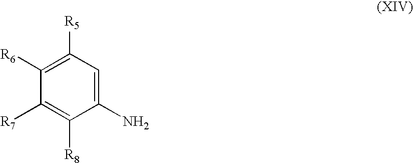
wherein R₅, R₆, R₇ and R₈ are as defined above for a compound of formula (I), to obtain a compound of formula (VI), and optionally forming enantiomers, racemates, diastereomers, or pharmaceutically acceptable salts of the compounds of formula (XIV).

33. An intermediate of formula (IV): 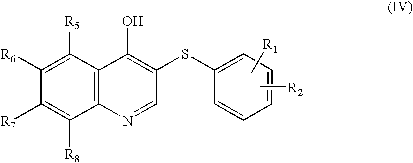
wherein:
R₁ and R₂ are independently selected from hydrogen, halogen, alkyl, alkoxy, cyano, an optionally substituted amino group, and a saturated heterocyclyl group containing N as a heteroatom; and,
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof.

34. An intermediate of formula (V): 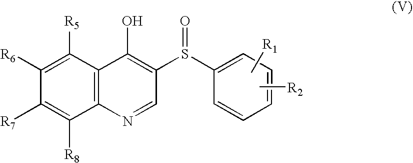
wherein:
R₁ and R₂ are independently selected from hydrogen, halogen, alkyl, alkoxy, cyano, an optionally substituted amino group, and a saturated heterocyclyl group, wherein the heteroatom is N; and,
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof.

35. An intermediate of formula (VI): 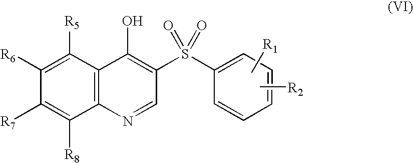
wherein:
R₁ and R₂ are independently selected from hydrogen, halogen, alkyl, alkoxy, cyano, an optionally substituted amino group, and a saturated heterocyclyl group containing N as a heteroatom; and,
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof.

36. An intermediate of formula (VII) 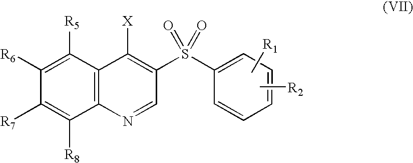
wherein X is selected from halogen, benzenesulphonyloxy and trifluoromethanesulphonyloxy;
R₁ and R₂ are independently selected from and a hydrogen, halogen, alkyl, alkoxy, cyano, an optionally substituted amino group, and a saturated heterocyclyl group containing N as a heteroatom; and,
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof.

37. An intermediate of formula (XVI): 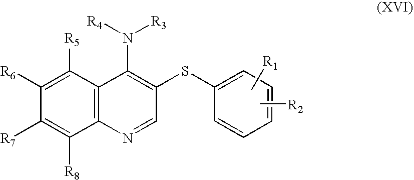
wherein:
R₁ and R₂ are independently selected from hydrogen, halogen, alkyl, alkoxy, cyano, an optionally substituted amino group, and a saturated heterocyclyl group containing N as a heteroatom;
R₃ and R₄ are independently selected from hydrogen, alkyl, and an aryl group optionally substituted with at least one substituent selected from halogen, alkyl, and an alkoxy group, or
R₃ and R₄ together with the N atom to which they are attached form a C₅₋₇ heterocyclyl group containing 1 or 2 heteroatom(s) selected from the group of N and O, and which heterocyclyl group may be optionally substituted by a substituent selected from halogen, alkyl, alkylhydroxy, alkyloxycarbonyl, aminocarbonyl, —OCH2CH2O—, benzyl and a substituted phenyl group; and,
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof.

38. An intermediate of formula (XVII): 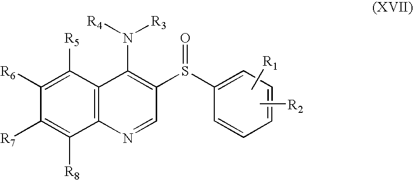
wherein:
R₁ and R₂ are independently selected from hydrogen, halogen, alkyl, alkoxy, cyano, an optionally substituted amino group, and a saturated heterocyclyl group containing N as a heteroatom;
R₃ and R₄ are independently selected from hydrogen, alkyl, and an aryl group optionally substituted with at least one substituent selected from halogen, alkyl, and an alkoxy group, or
R₃ and R₄ together with the N atom to which they are attached form a C₅₋₇ heterocyclyl group containing 1 or 2 heteroatom(s) selected from the group of N and O, and which heterocyclyl group may be optionally substituted by a substituent selected from halogen, allyl, alkylhydroxy, alkyloxycarbonyl, aminocarbonyl, —OCH₂CH₂O—, benzyl and a substituted phenyl group; and,
R₅, R₆, R₇ and R₈ are independently selected from hydrogen, halogen, alkyl, alkoxy, and a cyano group, or R₆ and R₇ together with the atoms to which they are attached form an unsaturated heterocyclyl group;
or enantiomers, racemates, diastereomers, or pharmaceutically acceptable salts thereof.

39. A pharmaceutical formulation comprising as an active ingredient a therapeutically effective amount of a compound of formula (I): 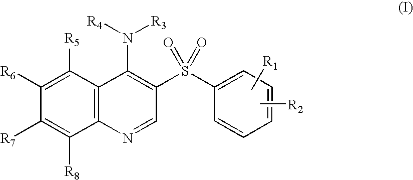
as defined in claim 27 with at least one of a pharmaceutically acceptable diluent, excipient, or inert carrier.

40. A pharmaceutical formulation comprising as an active ingredient a therapeutically effective amount of a compound of formula (I): 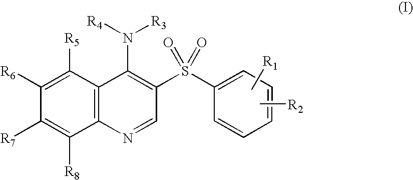
as defined in claim 28 with at least one of a pharmaceutically acceptable diluent, excipient, or inert carrier.

41. A pharmaceutical formulation comprising as an active ingredient a therapeutically effective amount of a compound of formula (I): 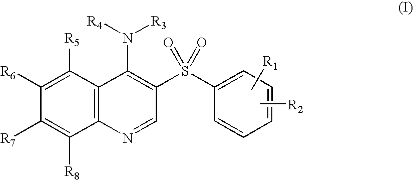
as defined in claim 29 with at least one of a pharmaceutically acceptable diluent, excipient, or inert carrier.

42. A method for treating mGluR5 receptor-mediated disorders, comprising administering to a mammal in need of such treatment the pharmaceutical composition of claim 39.

43. A method for treating mGluR5 receptor-mediated disorders, comprising administering to a mammal in need of such treatment the pharmaceutical composition of claim 40.

44. A method for treating mGluR5 receptor-mediated disorders, comprising administering to a mammal in need of such treatment the pharmaceutical composition of claim 41.

45. A method treating mGluR5 receptor-mediated disorders, comprising administering to a mammal in need of such treatment, a therapeutically effective amount of a compound of formula (I) 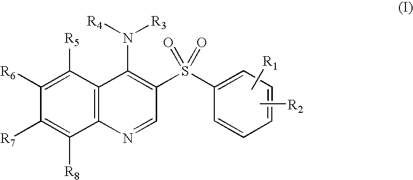
as defined in claim 27.

46. A method treating mGluR5 receptor-mediated disorders, comprising administering to a mammal in need of such treatment, a therapeutically effective amount of a compound of formula (I): 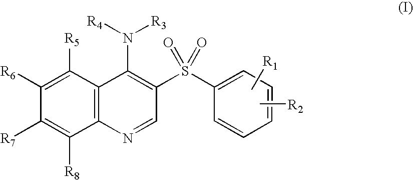
as defined in claim 28.

47. A method treating mGluR5 receptor-mediated disorders, comprising administering to a mammal in need of such treatment, a therapeutically effective amount of a compound of formula (I): 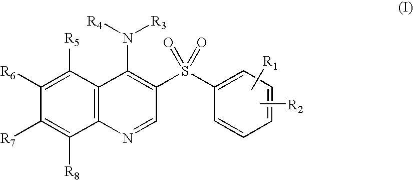
as defined in claim 29.

48. A method according to claim 45, wherein said mammal is a human.

49. A method according to claim 45, wherein said mGluR5 receptor-mediated disorders are psychiatric disorders.

50. A method according to claim 45, wherein said mGluR5 receptor-mediated disorders are neurological disorders.

51. A method according to claim 45, wherein said mGluR5 receptor-mediated disorders are chronic and acute pain disorders.

52. A method according to claim 45, wherein said mGluR5 receptor-mediated disorders are neuromuscular dysfunctions of the lower urinary tract and gastrointestinal disorders.
